SEQUENCE LISTING (63 sequences)

SEQ ID NO: 1 - DNA, 150 bp, Zea mays
  source 1..150: mol_type = genomic DNA
tctatgtattaatagaatctatagtattcttatagaataagaaaaaaaaaatgaagataataaactgcggattctttctttctcttccattcttacgtttccatattaaagtgtagtttttttacttaaatttaataatattaatctacc

SEQ ID NO: 2 - DNA, 173 bp, Zea mays
  source 1..173: mol_type = genomic DNA
ctcggatcctctatgtattaatagaatctatagtattcttatagaataagaaaaaaaaaatgaagataataaactgcggattctttctttctcttccattcttacgtttccatattaaagtgtagtttttttacttaaattttgtttaactttaagaaggagatatacatatg

SEQ ID NO: 3 - DNA, 230 bp, Zea mays
  source 1..230: mol_type = genomic DNA
actcccccaccacgatcgaacgggaatggataggaggcttgtgggattgacgtgatagggtagggttggctatactgctggtggcgaactccaggctaataatctgaagcgcatggatacaagttatccttggaaggaaagacaattccggatcctgtagaaataattttgtttaactttaagaaggagatatacccatgggtaaaggagaagaacttttcactggaatg

SEQ ID NO: 4 - DNA, 311 bp, Zea mays
  source 1..311: mol_type = genomic DNA
ctcactcccccaccacgatcgaacgggaatggataggaggcttgtgggattgacgtgatagggtagggttggctatactgctggtggcgaactccaggctaataatctgaagcgcttggatacaagttatccttggaaggaaagacaattccggatcctctatgtattaatagaatctatagtattcttatagaataagaaaaaaaaaatgaagataataaactgcggattctttctttctcttccattcttacgtttccatattaaagtgtagtttttttacttaaatttaataatattaatctaccatg

SEQ ID NO: 5 - DNA, 148 bp, Zea mays
  source 1..148: mol_type = genomic DNA
tagatttagggatttttcgagttgattcattccaccgcgaagtcccctgcatgggtatctaggaaatagttacttccaagtgaatcttccctagatacctaaaatctattttattatgatccatttcgcgaaaatatagattaagcta

SEQ ID NO: 6 - DNA, 150 bp, Nicotiana tabacum
  source 1..150: mol_type = genomic DNA
agaaattcaattaaggaaataaattaaggaaatacaaaaaggggggtagtcatttgtatataactttgtatgacttttctcttctatttttttgtatttcctccctttccttttctatttgtatttttttatcattgcttccattgaatt

SEQ ID NO: 7 - DNA, 201 bp, Nicotiana tabacum
  source 1..201: mol_type = genomic DNA
tgtctagagcgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgat

SEQ ID NO: 8 - DNA, 224 bp, Escherichia coli
  source 1..224: mol_type = genomic DNA
atcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaagct

SEQ ID NO: 9 - protein, 257 aa, Fischerella musicola
MLTNNEIERLRQAIIATIASPVIGSIEDYTWEAIFHYVKDIPLSDPALGRSKLLYDAVDVVTKTGWSLKSLQLKSLNFKSPFLFVIQRADILKKSVQLGFPGLTEQSSPDELGAAIIQHWNEKIILSQAAQSVVNSYEGILLKTIKGYEYIYCEFPLDPLDPSTFSWAWTVDKTTGGAGVGLQGSIVGKTELVWYKNQKQLFRARTIPAQAVRITVERTRLTLDRYVKTVIFALQDQINMQFSENEPEEPYDVPDYA

SEQ ID NO: 10 - DNA, 774 bp, Synthetic construct
  source 1..774: mol_type = other DNA
atgctgaccaataatgaaatagaaaggctgagacaggcgatcattgccaccatcgcatcgcctgtcattggttcgattgaggactatacttgggaggctatctttcactatgtcaaagatatacctctctctgatccagctctcggcagaagcaaactcctctatgacgctgtcgacgtggttacgaagacgggttggtcgctgaagtctcttcagctcaagtccttgaacttcaaatctccttttctctttgtcatacaaagggctgatattctgaaaaaatccgtccaactgggattcccaggtctgacagagcaaagcagcccagatgaactcggtgctgcgataatacaacattggaatgaaaagattatactctcgcaagccgcgcagtccgtggttaacagctatgaagggattcttctcaaaacaataaagggttacgagtacatttattgcgagttccctctcgatcctctggacccaagcacattttcgtgggcttggaccgtggacaagacgacggggggtgcgggagttggtttgcagggatcaatcgttggaaagacggagctggtttggtacaagaaccagaagcaactttttcgggcgaggaccattcctgcacaagccgttagaataacggtggaacgcaccaggctgacgctcgacaggtacgttaaaacagtgatctttgccttgcaggatcagatcaatatgcaattttcggagaacgagccggaagagccttatgatgttcctgattatgcttaa

SEQ ID NO: 11 - protein, 340 aa, Chlamydomonas reinhardtii
MTTKKTIQFYANIAQVRNHELISLSNIQTPSQGSITIFCKTCKTSFTTTARSYQNARKTGCPQCKAKTTSENWKGKIRTKSPEEASKQAVLNEYKQQKHLQKGLAYAHISNKEDLKIFLKESPNVYNNFILQRIDHPVIGKYTENHHIIPKHTGGPHKRWNLIKLTPEDHMEAHRLRALVYNEAGDHQAIRFRTNPSELVERRLRGNQIANETRLRERTGIYAEGASSKGGRIGGLVKSHEKDLKQSTKMSQPVLKALYEGSRWKHLQSGTELNLQPNRLFTLPQLVQKLLEALPPCKDKEVLERAKTSTVTSNLARVIKKQRPSAYGWITFPYDVPDYA

SEQ ID NO: 12 - DNA, 1023 bp, Synthetic construct
  source 1..1023: mol_type = other DNA
atgacgacaaagaaaacaatccagttctacgctaatatcgcgcaagtgcgcaaccacgagcttatttctttgtcaaacattcagaccccgagccaaggtagcatcacaattttctgtaagacttgcaaaacatcattcacgactacggctaggtcttatcagaacgcacggaaaactgggtgccctcagtgcaaggctaagacaacctcggaaaactggaaaggtaaaatacggacgaaatcccctgaagaggcctcgaagcaggcagtgctcaatgagtataaacaacaaaaacatttgcaaaagggtcttgcctatgcccacatttcaaataaagaagatctgaaaatctttttgaaggaatcgccgaatgtctataacaattttatacttcagagaatcgatcatccggtgattgggaaatatacagagaaccaccacattatcccaaaacacactggtgggccgcacaaaaggtggaatctgattaaattgacgccggaggaccatatggaagcgcatcgccttagggcactcgtgtataatgaagcgggcgaccatcaggcaatacgcttcaggaccaatcctagcgagctggttgagcggaggcttagaggaaaccaaatcgctaacgagacgagactcagggagaggacgggtatttatgcggagggggcatcatccaagggtgggagaataggtggactggtcaaatctcacgagaaagacctgaaacaaagcaccaaaatgagccagcccgttctgaaagcgttgtacgaaggctcgagatggaagcaccttcaatccggcacagagcttaacctgcaacccaaccggctctttactctcccccagctcgtgcagaagctgttggaagctcttccgccctgcaaagataaagaggtcctggaacgggcaaagacttcaactgtgacctccaatctggctcgcgtgatcaaaaaacagaggcctagcgcctatggatggattacgttcccttatgatgttcctgattatgcttaa

SEQ ID NO: 13 - DNA, 6661 bp, Synthetic construct
  source 1..6661: mol_type = other DNA
gcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatctggaattcccgggtaccggatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatgcagtctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatcgcgcaatgcactgacgggtgcccccctgaacctgaccccggcacaggtggtggccatcgccagcaataacggcggcaagcaggcactagaaacggtgcagcggctgttgccggtgctgtgccaggaccatggcctgaccccggaccaggtggtggccatcgccagcaatattggcggcaagcaggctcttgaaacggtgcagcggctgttgccggtgctgtgccaggcccatggcctgaccccggaccaagtggtggccatcgccagcaatggcggcggcaagcaggcgttggaaacggtgcagcggctgttgccggtgctgtgccaggaccatggcctgaccccggaccaggtcgtggccatcgccagcaatattggcggcaagcaggcgctcgaaacggtgcagcggctgttgccggtgctgtgccaggcccatggcctgaccccggaccaagtggtggccatcgccagcaatggcggcggcaagcaggcagttgaaacggtgcagcggctgttgccggtgctgtgccaggaccatggcctgaccccggaccaagtcgtggccatcgccagccacgatggcggcaagcaggctgtagaaacggtgcagcggctgttgccggtgctgtgccaggcccatggcctgaccccggcccaagtggtggccatcgccagcaatggcggcggcaagcaggccgtggaaacggtgcagcggctgttgccggtgctgtgccaggcccatggcctgaccccggaccaagtggtggccatcgccagcaatggcggcggcaagcaggcactggaaacactgcagcggctgttgccggtgctgtgccaggaccatggcctgaccccggaccaagtggtggccatcgccaacaataacggcggcaagcaggcactagaaacggtgcagcggctgttgccggtgctgtgccaggaccatggcctgaccccggaccaagtggtggccatcgccaacaataacggcggcaagcaggctcttgaaacggtgcagcggctgttgccggtgctgtgccaggaccatggcctgaccccggaccaagtcgtggccatcgccagccacgatggcggcaagcaggcgttggaaacggtgcagcggctgttgccggtgctgtgccaggaccatggcctgaccccggaccaggtcgtggccatcgccagcaatattggcggcaagcaggcgctcgaaacggtgcagcggctgttgccggtgctgtgccaggaccatggcctgaccccggaccaagtggtggccatcgccaacaataacggcggcaagcaggcagttgaaacggtgcagcggctgttgccggtgctgtgccaggaccatggcctgaccccggaccaagtcgtggccatcgccagccacgatggcggcaagcaggctgtagaaacggtgcagcggctgttgccggtgctgtgccaggaccatggcctgaccccggaccaggtcgtggccatcgccagcaatattggcggcaagcaggccgtggaaacggtgcagcggctgttgccggtgctgtgccaggcccatggcctgaccccggcccaagtggtggccatcgccagcaatggcggcggcaagcaggcgctggaaactgtacagcggctgttgccggtgctgtgccaggcccatggcctgaccccggaccaagtggtggccatcgccagcaatggcggcggcaagcaggcactagaaacggtgcagcggctgttgccggtgctgtgccaggaccatggcctgaccccggaccaggtggtggccatcgccagccacgatggcggcaagcaggctcttgaaacggtgcagcggctgttgccggtgctgtgccaggaccatggcctgaccccggaccaagtcgtggccatcgccagccacgatggcggcaagcaggcgttggaaacggtgcagcggctgttgccggtgctgtgccaggaccatggcctgaccccggaccaagtggtggccatcgccaacaataacggcggcaagcaggcgctcgaaacggtgcagcggctgttgccggtgctgtgccaggaccatggcctgaccccggaccaggtcgtggccatcgccagcaatattggcggcaagcaggcagttgaaacggtgcagcggctgttgccggtgctgtgccaggaccatggcctgaccccggaccaagtggtggccatcgccaacaataacggcggcaagcaggctgtagaaacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatggccgctgccaaaccgtcccctacttcagctcaaacaccggatcaggcgtctttccatgcagattacaaggacgacgacgacaagaagggtcgacccagcccaatgcacgagggagatcagacgcgggcaagcagccgtgccgcttcccgatcggatcgtgctgtcaccggcccctccacacagcaatctttcgaggtgcgcgttcccgaacagcaagatgcgctgcatttgcccctcagctggagggtagctgctccgcgtaccaggatcgggggcggcctcccggatcctggtacgcccatcgctgccgacctggcagcgtccagcaccgtgatcagatccggctcctccggctacgcgctggggccttaccagataagcgcacctcagttgcctgcgtataatggtcagacagtgggcacgttttattatgtcaatgacgccggaggtcttgaatccaaggtgttctcttcagggggtccaacaccgtatcctaattatgccaacgccggccacgtggagggacagtcagcattgtttatgagggacaacggtatctcggaaggtcttgtgttccataataatccagaaggcacctgtggcttctgtgtcaatatgacagagactcttcttcccgaaaacgcaaagatgaccgtggttcctcccgagggaggctcctccggcacgaacctgtctgacattatcgagaaggaaacaggtaagcaacttgttatacaagagtctatacttatgcttcctgaggaggtcgaggaggtcattggcaataagcccgaatccgacatactcgtccacacagcctacgacgaatcgacggacgaaaatgtgatgcttctcacgtctgatgcgcctgaatacaaaccgtgggcacttgttattcaagactcaaacggggagaacaaaataaaaatgctctaaactagtgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatac

SEQ ID NO: 14 - DNA, 6422 bp, Synthetic construct
  misc_feature 4365..4371: note = n is a, c, g, or t
  source 1..6422: mol_type = other DNA
attcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaatactagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatccgaattcggtaccagatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatgcagtctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatcgcgcaatgcactgacgggtgcccccctgaacctgaccccggcacaggtggtggccatcgccagcaataacggcggcaagcaggcactagaaacggtgcagcggctgttgccggtgctgtgccaggaccatggcctgaccccggaccaggtggtggccatcgccagccacgatggcggcaagcaggctcttgaaacggtgcagcggctgttgccggtgctgtgccaggaccatggcctgaccccggaccaggtggtggccatcgccagcaatattggcggcaagcaggcgttggaaacggtgcagcggctgttgccggtgctgtgccaggaccatggcctgaccccggaccaagtggtggccatcgccaacaataacggcggcaagcaggcgctcgaaacggtgcagcggctgttgccggtgctgtgccaggaccatggcctgaccccggaccaagtcgtggccatcgccagccacgatggcggcaagcaggcagttgaaacggtgcagcggctgttgccggtgctgtgccaggcccatggcctgaccccggcccaagtggtggccatcgccagcaatggcggcggcaagcaggctgtagaaacggtgcagcggctgttgccggtgctgtgccaggaccatggcctgaccccggaccaagtcgtggccatcgccagccacgatggcggcaagcaggccgtggaaacggtgcagcggctgttgccggtgctgtgccaggaccatggcctgaccccggaccaggtggtggccatcgccagccacgatggcggcaagcaggcactggaaacactgcagcggctgttgccggtgctgtgccaggcccatggcctgaccccggcccaagtggtggccatcgccagcaatggcggcggcaagcaggcactagaaacggtgcagcggctgttgccggtgctgtgccaggaccatggcctgaccccggaccaagtggtggccatcgccaacaataacggcggcaagcaggctcttgaaacggtgcagcggctgttgccggtgctgtgccaggaccatggcctgaccccggaccaagtcgtggccatcgccagccacgatggcggcaagcaggcgttggaaacggtgcagcggctgttgccggtgctgtgccaggcccatggcctgaccccggcccaagtggtggccatcgccagcaatggcggcggcaagcaggcgctcgaaacggtgcagcggctgttgccggtgctgtgccaggcccatggcctgaccccggaccaagtggtggccatcgccagcaatggcggcggcaagcaggcagttgaaacggtgcagcggctgttgccggtgctgtgccaggaccatggcctgaccccggaccaagtcgtggccatcgccagccacgatggcggcaagcaggctgtagaaacggtgcagcggctgttgccggtgctgtgccaggcccatggcctgaccccggcccaagtggtggccatcgccagcaatggcggcggcaagcaggccgtggaaacggtgcagcggctgttgccggtgctgtgccaggcccatggcctgaccccggcccaagtggtggccatcgccagcaatggcggcggcaagcaggcgctggaaactgtacagcggctgtnnnnnnngctgtgccaggaccatggcctgaccccggaccaagtcgtggccatcgccagccacgatggcggcaagcaggcactagaaacggtgcagcggctgttgccggtgctgtgccaggaccatggcctgaccccggaccaggtggtggccatcgccagcaatattggcggcaagcaggctcttgaaacggtgcagcggctgttgccggtgctgtgccaggaccatggcctgaccccggaccaagtggtggccatcgccaacaataacggcggcaagcaggcgttggaaacggtgcagcggctgttgccggtgctgtgccaggaccatggcctgaccccggaccaagtggtggccatcgccaacaataacggcggcaagcaggcgctcgaaacggtgcagcggctgttgccggtgctgtgccaggaccatggcctgaccccggaccaagtcgtggccatcgccagccacgatggcggcaagcaggcagttgaaacggtgcagcggctgttgccggtgctgtgccaggaccatggcctgaccccggaccaagtggtggccatcgccaacaataacggcggcaagcaggctgtagaaacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatggccgctgccaaaccgtcccctacttcagctcaaacaccggatcaggcgtctttccatgcagattacaaggacgacgacgacaagaagggtcgacccagcccaatgcacgagggagatcagacgcgggcaagcagccgtgccgcttcccgatcggatcgtgctgtcaccggcccctccacacagcaatctttcgaggtgcgcgttcccgaacagcaagatgcgctgcatttgcccctcagctggagggtagctgctccgcgtaccaggatcgggggcggcctcccggatcctggtacgcccatcgctgccgacctggcagcgtccagcaccgtgatcagatccggctcctccggcgcaatccccgttaagcgcggggcaaccggggaaactaaggttttcacgggtaattcaaattcacctaagtccccgactaaaggaggatgcggctcctccggcacgaacctgtctgacattatcgagaaggaaacaggtaagcaacttgttatacaagagtctatacttatgcttcctgaggaggtcgaggaggtcattggcaataagcccgaatccgacatactcgtccacacagcctacgacgaatcgacggacgaaaatgtgatgcttctcacgtctgatgcgcctgaatacaaaccgtgggcacttgttattcaagactcaaacggggagaacaaaataaaaatgctctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 15 - DNA, 3895 bp, Synthetic construct
  source 1..3895: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaattttttagtacatctattttattctattttagcctctaaattaagaactaactctattttagtttttttatttaataatttagatataaaatagaataaaatagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccttctacttctctagatccttccggtccatggttaggcccggtagttctacttctgttcatttgtgttagatccgtgtttgtgttagatccgtgctgctagcgttcgacggagcgacctgtacgtcagacacgttctgattgctaacttgccagttttctcttggggaatcctgggatggctctagccgttccgcagacgggatcgatttcatgatttttttgtttcgttcatagggtttggtttccctttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggacttatgttggccatacatattcatagttacgaattgaagagaggatggaaatatcgatctaggataggtaacatgttgatgcgggtttactgatgcaatacagagatgcttgtcgcttggtgtgatatgtggttggtgggcggtcgtcattcgtctagatcggagtagaatactgttcaaactacctggtgtatttataatttggaactgtatgtgtgtgtcaacatcttcatagtacgagttaagatggaggaaatacgatcagaaggtatacatgtgatgtgggtacgatgcaatacatgatggcatatgcagcatctattcatatgctctaaccttgagtacctatctattataataaacaagtatgtttataattattttgacttgatatacttggatatggcatatgcagcagctatatgggattttttagccctgccttcatacgctatttatttgcttggtacttttcttttgtcgatgctcaccctgttgttggtgttacttctgcagggcgcgcctcgactctagaggatcgatccccgggatggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgggatccatgtctccccaaactgaaacaaaagctagcgttgggtttaaagcgggcgttaaagactataaactgacttactacaccccggaatacgaaacgaaggacacagatatacttgccgcgttccgggtgacgccccaactcggcgtgccgccagaagaggccggtgcggctgttgcggctgaatcatccactggaacatggaccacagtgtggacggatgggttgacctctttggataggtataagggacggtgttatcatatcgagcccgttccgggcgaccctgatcagtacatttgttacgtggcgtaccccttggatttgttcgaagaaggatcggtgacaaacatgtttacatccatagtcggtaacgtcttcgggttcaaagcgcttagggcgctgagacttgaagatctgagaataccgccggcatattcgaagacattccagggtcctccccacggaatccaggtggaaagggataagctcaataaatatgggagaccactcttgggctgtacaatcaaaccgaaactggggctgagcgccaagaattatgggagggcctgttacgaatgcttgcgcgggggtctggatttcactaaggatgacgaaaacgtcaatagccagccttttatgcgctggcgcgaccgcttcgtcttctgcgctgaagcgatctacaaagcgcaagccgagacaggagaaataaaaggacactacctgaatgcaaccgcagggacgtgtgaagagatgatcaagcgggccgtgtttgcacgggagctgggagtgcccatagttatgcacgattaccttacgggaggattcactgcgaacactacattgagccattactgtagagataacggactcttgttgcacatacaccgcgcgatgcacgccgttatcgaccggcaaaaaaaccacgggatgcatttcagggttcttgcaaaggcactgaggatgtctgggggcgaccatatccactcgggtacggtggtggggaagctcgagggagaaagggaaataacactcggtttcgttgacctcctgcgcgacgacttcattgagaaagaccgctcgcgcgggatctttttcactcaggattgggtgtcaatgccgggcgtcatacccgtggcgtcaggaggtatacatgtgtggcacatgccggcactcaccgaaatatttggggatgatagcgtgctccagtttggcggtgggacgttgggtcatccctggggtaatgcccccggtgctgctgccaacagggtcgcacttgaagcttgcgtccaggcgcggaatgaggggcgggatcttgccagagaagggaacgaaatcattaaggcagcttgcaaatggagcgccgaactcgcggcagcgtgtgaaatctggaaagagatcaagttcgacgggtttaaagcgatggatacaatttaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 16 - DNA, 3961 bp, Synthetic construct
  source 1..3961: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaattttttagtacatctattttattctattttagcctctaaattaagaactaactctattttagtttttttatttaataatttagatataaaatagaataaaatagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccttctacttctctagatccttccggtccatggttaggcccggtagttctacttctgttcatttgtgttagatccgtgtttgtgttagatccgtgctgctagcgttcgacggagcgacctgtacgtcagacacgttctgattgctaacttgccagttttctcttggggaatcctgggatggctctagccgttccgcagacgggatcgatttcatgatttttttgtttcgttcatagggtttggtttccctttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggacttatgttggccatacatattcatagttacgaattgaagagaggatggaaatatcgatctaggataggtaacatgttgatgcgggtttactgatgcaatacagagatgcttgtcgcttggtgtgatatgtggttggtgggcggtcgtcattcgtctagatcggagtagaatactgttcaaactacctggtgtatttataatttggaactgtatgtgtgtgtcaacatcttcatagtacgagttaagatggaggaaatacgatcagaaggtatacatgtgatgtgggtacgatgcaatacatgatggcatatgcagcatctattcatatgctctaaccttgagtacctatctattataataaacaagtatgtttataattattttgacttgatatacttggatatggcatatgcagcagctatatgggattttttagccctgccttcatacgctatttatttgcttggtacttttcttttgtcgatgctcaccctgttgttggtgttacttctgcagggcgcgcctcgactctagaggatcgatccccgggatggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgggatccatgcggacaaatccaacaacatcgaggcctgggatctccacgattgaagaaaagtccgttggaaggatcgatcaaattattggcccggtgctcgacataaccttcccgcccggaaagttgccatacatatataatgccttgatcgtgaagagccgcgacacagcagacaagcagataaacgtgacatgtgaagtccagcagttgctggggaacaatagagtgcgggcggtggccatgtcagctacggaaggccttatgcgcggaatggaggttatcgacacgggtactcccttgagcgtccccgttggcggtgctactcttggtcgcatttttaacgtgctgggggaacctatagacaatcttggtcccgttgatacctctgcgactttccccattcatcggtcggcaccggccttcatcgagcttgatactaagctctccatatttgaaactggaataaaagttgtggacctgctggctccgtacaggaggggaggtaagataggcctttttggtggggccggggttggaaagacagtgctgattatggaactcataaataacattgcaaaagcacacggtggagtgtctgtctttggtggcgtgggagagagaacccgggaagggaatgacctgtacatggaaatgaaagagtcgggcgttatcaacgagaaaaacatcgaagagtccaaggtcgccctcgtgtatggacagatgaacgagccgcccggtgctcggatgcgcgtgggtctcacggcgttgactatggccgagtactttagagatgtcaacaaacaagatgtgcttctttttatagataatatatttcgcttcgttcaggctggatctgaagtttcggccctccttggccggatgcccagcgcggtcggttaccagcctacactctcaactgaaatggggtctttgcaagagagaattaccagcacaaagaaaggttctataacttctattcaggccgtttatgtcccagcggacgacctgacggacccggcacccgccacgacgttcgcccatcttgatgcgacaacggtgctttcacggggcctcgcaagcaagggaatatatccggctgtggaccctcttgattccacatctaccatgctgcagccgcgcatagtcggcaatgaacactacgaaacggcgcagagagtgaaagagacactccaacgctataaagagttgcaggatataattgctattctgggtcttgacgaactctccgaagaggaccggctcactgtggcgcgggcacggaagatagagcgcttcttgagccaacccttctttgttgcagaagttttcaccggaagccctggtaagtacgtcggccttgctgagaccatccgcggatttcagctcattctctcgggcgaattggacggacttcccgagcaggcattttacctggtcgggaacatagatgaagcatccacaaaggcaatcaatttggaggaggaatcaaagttgaagaaatgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 17 - DNA, 5916 bp, Synthetic construct
  source 1..5916: mol_type = other DNA
attcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaatactagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatccgaattcggtaccagatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatgcagtctgccaccatggatcccattcgttcgcgcacgccaagtcctgcccgcgagcttctgcccggaccccaaccggatagggttcagccgactgcagatcggggaggcgctccacccgcgggcgggccccttgatggtttgccagcaagaagaactatgtctagaacaagattgccatctccaccagccccttcgcccgccttctcagctggttcgttttcggacttgttgcggcaatttgatccttcgctgcttgacactagcctcttggactcgatgcctgctgtcgggacacctcatacggcagctgcgccggcagaatgggacgaggtgcagtccggtttgcgggcagccgacgacccgcctcccacagtgagagttgcagttacagctgcaagacctccaagagccaaacctgctcccaggagacgcgctgcccaaccatccgacgcttcgccggccgcgcaggtggatcttcgcacccttgggtactcacaacagcagcaagagaaaataaagcctaaggtccggtcgacagttgcacagcatcacgaagcactggtggggcatggtttcacacatgcccatatagttgccttgagccagcaccccgccgcgctcggcaccgtggccgtcaaatatcagcatatcatcactgcactcccggaagcaactcatgaagacatagtcggggttggtaagcaatggtcaggagcccgcgcgctggaggcgcttctgacgaaggccggcgaacttcgcgggccaccactccagctggatactggccagttgcttaagatagctaagagaggcggcgtgacggcggtcgaagctgtgcacgcctggaggaatgctcttacgggggctccactgaacttgactcccgaccaagtggttgcgattgcatccaacataggtgggaagcaggcactcgaaaccgttcagagattgcttccggttttgtgccaggatcatggactcactcccgatcaagttgttgcaatagcgtctcatgatggcggcaagcaggctctggagaccgtgcagagacttctgccggttctttgccaagatcatggcctgacaccagaccaggtcgttgcgatagcaagcaataatggaggtaagcaggcgcttgagactgtgcaaagactcctcccagtgctctgtcaagaccacggtttgacgccggaccaggtcgtcgcaatcgcgtctaatattgggggcaagcaagcgctggagacggttcagcggcttttgccggtcctgtgccaggaccatggcttgacgccagaccaggtggttgcaatcgcgagccatgacggcggcaagcaggcgctggagacggttcagcgcctccttccagttttgtgccaggaccatgggctcactcccgatcaggttgtcgctatagccagcaataatggcgggaaacaggcactcgaaacagtgcagaggcttctccccgttctgtgtcaggaccatggtttgacgcccgaccaggtggtcgctatcgcctccaatatagggggtaagcaggccttggagacggtgcaacggctgttgccagttctctgccaagaccatggcctgacaccggatcaagtggtggccatcgcctcgaataatggggggaagcaagccctcgaaaccgtccaaaggttgctccccgtcctctgtcaggaccatggtctgactcctgaccaggtcgtggcgatcgcgtcaaacggcggcggtaagcaggctttggagacagttcagaggttgctccccgtcttgtgccaagatcacggactgactccggaccaagtcgtcgcaatcgcgagcaatatcggtggcaaacaagctctcgagacagtccaaagacttttgccggtcctctgccaagaccacggcttgacaccagaccaagtggttgcaattgcttcaaacaacggcgggaagcaggccctcgaaaccgtccagaggcttttgcccgttctctgccaagaccacggccttacgccagatcaagtggtcgccatcgcctccaacggaggcggcaagcaagctcttgaaacagtgcaaaggctcctgcccgttctctgccaagatcatggactcacccccgatcaggtcgttgccattgcctccaacaacggtggaaagcaggcccttgaaacggtgcaaaggctgcttccagtcctttgtcaggatcatggacttacacccgatcaggtggttgcaatagcttccaacaatggcggcaagcaagcgttggagactgtgcaacggttgcttcctgtcctttgtcaagaccacggcttgacaccagatcaggtcgttgcgattgcctcgaataatgggggtaagcaagcattggaaaccgtccagaggttgcttccagtcctctgtcaagatcatggtctcaccccggaccaggtggtggcgattgcgtctaacaatggtggaaagcaggcacttgaaacagtgcaaaggttgcttccggtcttgtgtcaggaccatggcctgacgccggaccaggtcgtcgcaatcgcatcaaatggtggtggtaagcaggctttggaaactgttcaaagactcttgccggtcctctgtcaggaccacggtctcactccggatcaggtggtcgccatcgcatctcacgatgggggcaaacaggctctcgaatctatcgttgcccagctttctagacccgaccctgcgctcgccgccctgacaaatgaccatctcgttgctcttgcgtgcctgggtgggcgcccggcgttggacgcggtcaaaaagggattgcctcatgcgcctgagctgataagaaggatcaacagacgcattccagaacggacctcacatcgggttccagacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatggccgctgccaaaccgtcccctacttcagctcaaacaccggatcaggcgtctttccatgcagattacaaggacgacgacgacaagaagggtcgacccagcccaatgcacgagggagatcagacgcgggcaagcagccgtgccgcttcccgatcggatcgtgctgtcaccggcccctccacacagcaatctttcgaggtgcgcgttcccgaacagcaagatgcgctgcatttgcccctcagctggagggtagctgctccgcgtaccaggatcgggggcggcctcccggatcctggtacgcccatcgctgccgacctggcagcgtccagcaccgtgatcagatccggctcctccggcgcaatccccgttaagcgcggggcaaccggggaaactaaggttttcacgggtaattcaaattcacctaagtccccgactaaaggaggatgcggctcctccggcacgaacctgtctgacattatcgagaaggaaacaggtaagcaacttgttatacaagagtctatacttatgcttcctgaggaggtcgaggaggtcattggcaataagcccgaatccgacatactcgtccacacagcctacgacgaatcgacggacgaaaatgtgatgcttctcacgtctgatgcgcctgaatacaaaccgtgggcacttgttattcaagactcaaacggggagaacaaaataaaaatgctctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatca

SEQ ID NO: 18 - DNA, 6338 bp, Synthetic construct
  source 1..6338: mol_type = other DNA
gcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatctggaattcccgggtaccggatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatgcagtctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcaccaaagcgggtgaacttagagggcccccactgcaactcgacacaggacagctcttgaagatcgccaaaagaggcggagtgacggctgttgaagccgttcatgcgtggagaaatgcccttacgggagcacctctcaatctgacccccgatcaggtcgtcgccatcgcctccaacggtggagggaagcaagcacttgaaaccgtccaaaggctgctcccggtgctttgccaggatcatggccttacccccgatcaggtcgtcgctatagcgtctaatatcggggggaaacaggccctcgagactgttcagcggcttctgccagtcctctgccaagaccatggtctcacgcctgatcaggtggtcgcgatagcgtctaacatcggaggaaaacaagccctcgagacggttcaaagactcctcccggtgctctgtcaagaccatgggttgacccctgatcaagtggttgcgatcgcatccaacaatgggggtaaacaagctctggagaccgtccagagactcctgccggtgctgtgccaagaccatggtttgactcctgatcaggtcgtcgccatagcctcgaacattggtggtaaacaggcactggaaacggtccaaagacttcttcctgttctctgtcaagatcatggccttacacctgatcaggttgtggccatagcgtcgaacggaggtggtaagcaggcactcgagacggtgcagagactcttgccagtcttgtgtcaggaccacggcttgactcctgaccaagtggtcgccatagcctcgaacggtgggggcaaacaagccctggaaactgtccaaagactcctcccggtcctctgtcaggatcatggtcttaccccagatcaggtggtcgccattgcatcgaacggcggcggtaaacaggccctggaaacggtccaaagattgctcccggtgttgtgccaagaccacggcttgacacccgaccaagtggtggctatagcatcccacgacggcggtaaacaagcgctcgagaccgttcagcgcctgctgccggtgctctgccaagatcatggcctcactccggatcaggtggtcgcaatagcatcgcacgacggcgggaagcaggctttggagacagttcaacgcctgcttccggttctgtgccaagatcatggacttaccccagatcaagtggtcgccatcgcgtctcatgatggcggaaaacaggcgcttgaaacggtccagagattgttgccggtcctttgccaggaccatgggctcacgccggaccaagtcgtcgctatagcgtcgaacattggcggcaagcaggctttggagactgtgcagcgcttgctgccagttctttgtcaggatcatggtcttactcctgatcaagtggtggctatagcttccaataacggcggtaagcaggcgcttgaaactgtgcagcggctccttccagtcctgtgtcaggatcacggcctcactccagaccaagttgtggccatcgcgtctaacaatggggggaagcaagcactcgagacagtccagcgcctcctgcccgtcctctgtcaagaccacggccttacccctgatcaggtcgtcgctatcgcatccaacggaggcgggaaacaggccctcgagaccgttcagaggttgttgccggttctttgccaagaccacggtcttaccccagatcaggttgttgcgatcgccagcaacggcggtggcaagcaggctttggaaaccgttcaacggttgcttccagttttgtgtcaggatcatggcctcacccccgaccaagtcgtcgctattgcctcgaatggtggtgggaagcaggccctcgagacggttcagcggctgttgccagtgctctgccaagaccacggcctgacccctgatcaagtggtcgcgatcgcttcaaacataggagggaaacaggccctcgaaactgtccaaaggctgttgccagtcctctgccaggaccacggtctcacgccagatcaggtggttgccatagcatcaaacaacgggggtaaacaagcactggaaagcattgtcgctcaactgtcgaggccggatccagctcttgccgctttgaccaatgatcacttggtcgcattggcgtgccttggaggtcggccggcgctcgatgcggttaaaaaaggactcccacatgcacctgaactgattaggagaataaatcgcaggatacctgaaagaacttctcaccgcgttccagatcttgcacacgttgttcgggtgctcgggttctttcagtcacatagccaccccgctcaggcattcgatgacgcgatgactcagttcgagatgtcccgccacgggctggttcagctcttcaggcgggtgggagttaccgaattcgaagcccgctacggaacgctccccccagcctcgcagcgttgggaccgtatcctccaggcatcagggatggccgctgccaaaccgtcccctacttcagctcaaacaccggatcaggcgtctttccatgcagattacaaggacgacgacgacaagaagggtcgacccagcccaatgcacgagggagatcagacgcgggcaagcagccgtgccgcttcccgatcggatcgtgctgtcaccggcccctccacacagcaatctttcgaggtgcgcgttcccgaacagcaagatgcgctgcatttgcccctcagctggagggtagctgctccgcgtaccaggatcgggggcggcctcccggatcctggtacgcccatcgctgccgacctggcagcgtccagcaccgtgatcagatccggctcctccggctacgcgctggggccttaccagataagcgcacctcagttgcctgcgtataatggtcagacagtgggcacgttttattatgtcaatgacgccggaggtcttgaatccaaggtgttctcttcagggggtccaacaccgtatcctaattatgccaacgccggccacgtggagggacagtcagcattgtttatgagggacaacggtatctcggaaggtcttgtgttccataataatccagaaggcacctgtggcttctgtgtcaatatgacagagactcttcttcccgaaaacgcaaagatgaccgtggttcctcccgagggaggctcctccggcacgaacctgtctgacattatcgagaaggaaacaggtaagcaacttgttatacaagagtctatacttatgcttcctgaggaggtcgaggaggtcattggcaataagcccgaatccgacatactcgtccacacagcctacgacgaatcgacggacgaaaatgtgatgcttctcacgtctgatgcgcctgaatacaaaccgtgggcacttgttattcaagactcaaacggggagaacaaaataaaaatgctctaaactagtgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 19 - DNA, 4669 bp, Zea mays
  source 1..4669: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaattttttagtacatctattttattctattttagcctctaaattaagaactaactctattttagtttttttatttaataatttagatataaaatagaataaaatagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccttctacttctctagatccttccggtccatggttaggcccggtagttctacttctgttcatttgtgttagatccgtgtttgtgttagatccgtgctgctagcgttcgacggagcgacctgtacgtcagacacgttctgattgctaacttgccagttttctcttggggaatcctgggatggctctagccgttccgcagacgggatcgatttcatgatttttttgtttcgttcatagggtttggtttccctttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggacttatgttggccatacatattcatagttacgaattgaagagaggatggaaatatcgatctaggataggtaacatgttgatgcgggtttactgatgcaatacagagatgcttgtcgcttggtgtgatatgtggttggtgggcggtcgtcattcgtctagatcggagtagaatactgttcaaactacctggtgtatttataatttggaactgtatgtgtgtgtcaacatcttcatagtacgagttaagatggaggaaatacgatcagaaggtatacatgtgatgtgggtacgatgcaatacatgatggcatatgcagcatctattcatatgctctaaccttgagtacctatctattataataaacaagtatgtttataattattttgacttgatatacttggatatggcatatgcagcagctatatgggattttttagccctgccttcatacgctatttatttgcttggtacttttcttttgtcgatgctcaccctgttgttggtgttacttctgcagggcgcgcctcgactctagaggatcgatccccgggatggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgggatccatggaactcagattcccgagattttctcagggccttgctcaggaccccacgaccaggcgcatatggtttgggatagctactgctcacgactttgaatctcatgacgacattactgaagaacggctttaccaaaatatcttcgcttcccacttcgggcaacttgctattatattcttgtggactagcggtaacctctttcacgttgcatggcaaggtaacttcgaatcctggatccaagatccattgcacgttaggcccatagcccacgcaatatgggatcctcacttcggccagccagcagtggaggcatttacgcggggaggtgcagctggacccgtcaacattgcttattcaggcgtttatcagtggtggtatacgatagggcttcgcacaaatgaagatctctacaccggagctctttttctcctctttctgtccacgctttctttgatcgggggctggctgcaccttcaacccaagtggaaacctagcctttcctggtttaaaaacgcggaatcccgcctgaatcatcatctgtcaggcctcttcggagtgtcttctctggcttggacggggcacctcgttcacgtcgcaatccctggctcacggggcgagtacgtgcggtggaacaactttcttgatgtgctcccatatccgcagggattgggacccttgctgacaggtcaatggaacttgtatgcccaaaaccccgattcttcgaaccatcttttcggaactactcagggggctggtacggcaatcctgacgttgctggggggctttcatccgcagacgcagtccctctggcttactgatattgcccatcaccaccttgcaatcgcgtttatttttctcatagccggtcacatgtatcggactaacttcggtattgggcatagcattaaggatcttttggaggcacatacgccgccaggcgggaggttgggacgcgggcataaggggctttacgacaccatcaataactcgatccattttcaactcggactggcacttgcatctttgggggtgatcacaagcctcgtcgcgcagcacatgtattctttgcccgcatacgcgtttatcgcacaagacttcactactcaagccgctctgtatacgcaccatcaatatatcgctggatttatcatgacgggagcgttcgcccacggcgccattttcttcatcagagattataatccggaacagaacgaagacaatgttctcgcaaggatgcttgatcacaaagaagcgattatttcccatctctcttgggcgtcactgttcctgggtttccatactcttgggctctatgttcacaacgatgttatgctcgcatttggtacaccagagaaacagattcttatcgagcctattttcgctcaatggatccagtcagctcacgggaagacgacctacgggttcgatatactgctgtcgtcgaccaacggacccgcctttaacgcaggcagaaatatctggctcccaggatggctgaatgcggtcaacgaaaattctaactccttgttcctcaccataggtccaggggactttcttgttcatcacgccatcgccttgggattgcataccacaacgttgattcttgtcaaaggtgcactggatgcacgcggatcgaaacttatgccggataagaaagacttcggttacagcttcccctgcgatggcccgggacgcggcgggacttgcgatatttcagcctgggacgccttctacctggcggtgttctggatgttgaatacgattggttgggtgacattctattggcattggaagcacataacgctgtggcaggggaatgtttcacagtttaacgagtcgtccacatatctcatgggctggcttcgggattatttgtggcttaactcaagccagcttataaacggatacaacccctttgggatgaatagcttgagcgtgtgggcatggatgttccttttcggccacttggtgtgggcgactggattcatgtttctcatttcctggaggggttactggcaagaactgatcgagacactggcttgggctcatgaaagaactccacttgcgaacctgatcaggtggcgcgacaaaccagtcgcattgagcattgtgcaagcgaggctcgtgggactcgcccatttttcggtgggatacatatttacatacgccgcctttttgattgcttctacctctggaaagttcggataagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 20 - DNA, 4717 bp, Zea mays
  source 1..4717: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaattttttagtacatctattttattctattttagcctctaaattaagaactaactctattttagtttttttatttaataatttagatataaaatagaataaaatagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccttctacttctctagatccttccggtccatggttaggcccggtagttctacttctgttcatttgtgttagatccgtgtttgtgttagatccgtgctgctagcgttcgacggagcgacctgtacgtcagacacgttctgattgctaacttgccagttttctcttggggaatcctgggatggctctagccgttccgcagacgggatcgatttcatgatttttttgtttcgttcatagggtttggtttccctttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggacttatgttggccatacatattcatagttacgaattgaagagaggatggaaatatcgatctaggataggtaacatgttgatgcgggtttactgatgcaatacagagatgcttgtcgcttggtgtgatatgtggttggtgggcggtcgtcattcgtctagatcggagtagaatactgttcaaactacctggtgtatttataatttggaactgtatgtgtgtgtcaacatcttcatagtacgagttaagatggaggaaatacgatcagaaggtatacatgtgatgtgggtacgatgcaatacatgatggcatatgcagcatctattcatatgctctaaccttgagtacctatctattataataaacaagtatgtttataattattttgacttgatatacttggatatggcatatgcagcagctatatgggattttttagccctgccttcatacgctatttatttgcttggtacttttcttttgtcgatgctcaccctgttgttggtgttacttctgcagggcgcgcctcgactctagaggatcgatccccgggatggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgggatccatgattattcggtcttcggaaccggaggtgaaaatcgccgttgacagagaccccattaaaacttcattcgaagaatgggcgcggccgggacatttctcgcggaccatagctaaggggccggacacaacaacctggatttggaacctgcatgcggatgctcatgactttgattcccatactggtgacctggaagagattagccggaaagtgttctcggcccacttcggtcaattgtcaatcattttcctctggctctccgggatgtatttccacggtgcgcggttttccaattacgaagcgtggttgtctgatcccacgcatattggtccatccgctcaggtcgtgtggcctatagtggggcaagagatccttaatggtgatgtcggaggcggatttcgcgggatccaaataacgtcaggattcttccaaatctggcgggcgtccggtataacgtccgagctgcaattgtattgtaccgccataggcgctctcattttcgcctccctgatgttgttcgcagggtggtttcattatcacaaggcggctcctaaattggcatggttccaagacgtggaatctatgttgaaccatcacttggctggtctgttggggctgggatcccttagctgggcaggacaccaaattcacgtctcactccccataaatcagtttctggacgcaggggtcgatccgaaagaaatacccctgccccacgaattcattttgaatagggatctgctcgcgcagctctatccttcgttcgctgaaggagccaccccgttttttaccttgaattggtccaaatacgctgaatttctgagctttaggggcggcctggaccccattaccggcggtctctggctttcggatatcgctcaccatcaccttgctatcgctatcctgtttctgattgctggccatatgtaccggactaattggggcattgggcatggcctgaaggatattttggaggcgcacaaagggccgttcactggccaaggacataaggggctttacgaaatattgaccacgtcttggcatgcccagctttccctcaatctcgcgatgttgggatccactacgatcgtcgtggcccaccacatgtacagcatgcctccttatccatatctcgcgacagactatgggacccagctgtccctcttcacgcaccacatgtggataggcgggttcttgattgtgggcgctgcagcccacgccgctatcttcatggtgcgcgattacgatcctaccaccaggtataacgatctcttggatcgggtcctgcggcatcgcgatgccataatatcgcacctgaattgggtctgcatctttctcggctttcactcattcgggctgtatattcataacgacaccatgtcggcactcggtaggccacaagacatgttctccgatgcagccatccagctgcaacccatttttgcacagtggattcaaaacatacatgccggggctccaggcgtgaccgccccaggagcgactacatcgacctcacttacgtggggtggcggagaacttgtcgccataggaggcaaggttgcattgcttcctatacccctgggaaccgcagactttttggtgcatcacatccatgcttttaccattcatgtcacggttttgatattgctgaagggagtgctgtttgcaaggtcgtccaggctcattcctgataaagccaatctcggtttcagatttccatgtgatggtcctggccggggcggtacctgtcaggtctctgcgtgggatcatgttttcttgggtctcttttggatgtataactcgattagcgttgtgattttccatttttcctggaaaatgcaatctgacgtctggggcacgataagcgatcaaggcatagttacacacatcacaggtgggaatttcgcccagtcctcgataaccattaatggttggctgcgggatttcctgtgggcacaagcgtcccaagttattcaaagctatgggtcttcactgtctgcttatggacttttctttttgggcgcgcacttcgtgtgggccttctccttgatgttcttgttttccgggcgcgggtattggcaagagcttatcgagagcattgtctgggcccataataagctgaaagtggcaccagcgacgcaaccgcgggctctgtctattatacagggtcgcgccgtcggggtgacacattacctgttgggtggtattgccacgacgtgggcttttttcctcgccagaatcatcgcggttggctaggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 21 - DNA, 5692 bp, Synthetic construct
  source 1..5692: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaattttttagtacatctattttattctattttagcctctaaattaagaactaactctattttagtttttttatttaataatttagatataaaatagaataaaatagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccttctacttctctagatccttccggtccatggttaggcccggtagttctacttctgttcatttgtgttagatccgtgtttgtgttagatccgtgctgctagcgttcgacggagcgacctgtacgtcagacacgttctgattgctaacttgccagttttctcttggggaatcctgggatggctctagccgttccgcagacgggatcgatttcatgatttttttgtttcgttcatagggtttggtttccctttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggacttatgttggccatacatattcatagttacgaattgaagagaggatggaaatatcgatctaggataggtaacatgttgatgcgggtttactgatgcaatacagagatgcttgtcgcttggtgtgatatgtggttggtgggcggtcgtcattcgtctagatcggagtagaatactgttcaaactacctggtgtatttataatttggaactgtatgtgtgtgtcaacatcttcatagtacgagttaagatggaggaaatacgatcagaaggtatacatgtgatgtgggtacgatgcaatacatgatggcatatgcagcatctattcatatgctctaaccttgagtacctatctattataataaacaagtatgtttataattattttgacttgatatacttggatatggcatatgcagcagctatatgggattttttagccctgccttcatacgctatttatttgcttggtacttttcttttgtcgatgctcaccctgttgttggtgttacttctgcagggcgcgcctcgactctagaggatcgatccccgggatggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgggatccatgctgaggaacggcaatgaaggcatgtctaccataccggggttcagccagatccagttcgaggggttctgccggtttataaaccagggacttgccgaagaactggaaaaattccccacgataaaggacccagatcatgagatcgcttttcagttgtttgctaagggctatcagcttctggaaccatctataaaagaaaggaatgccgtttatgaaagcttgacttattcctcggagctgtacgtctctgctagattgatcttcgggtttgatgtccaaaaggaaacgatatctattggtaacataccgattatgaactctctgggcacgttcataataaatggaatctatagaatagttataaaccagatcctcctcagccctgggatctattatcgctcagaacttgaccataaaggtatctccatatacacaggaactataataagcgactggggtggtaggagcgaattggcgatagataaaaaagaacgcatttgggccagagtgtcgaggaagcagaaaattagcatccttgtccttagcagcgcaatgggatccaatcttcgggaaattcttgataatgtcagctaccccgagatcttcctgtcttttccaaacgcgaaagaaaaaaagcgcatagagtcaaaggaaaaagctattctggaattctaccagcaattcgcgtgcgtgggcggtgacctcgtgttctcagagtcattgtgtgaagagcttcaaaagaaatttttccagcagaagtgcgaactgggaagagtcgggagaagaaacatgaacagaagactgaacttggatataccccagaataatacgttcttgcttcctagagacgtcctggctgccacggatcacctgatcgggatgaaatttgggaccggtatacttgacgacgatgacatgaatcatctcaaaaataaaagaatacgctcggtcgctgaccttcttcaagaccagtttgggctcgctcttggacgcctgcaacacgccgttcaaaaaacaataagacgcgtttttattcggcagtcaaaacctactcctcaaaccctggttacaccgacctccacctccatccttcttataacaacatatgaaaccttttttggaacttaccccttggctcaggtttttgaccaaaccaatccacttacacagacagtccatggacggaaagtgtcctgcttgggaccaggcggattgacaggcagaacggcctcctttcggtcgagggacatacatccctcgcactacggccgcatatgtccgatagacacatcggaaggtattaacgtcggtctgacagggtctctcgccatacatgcaagaatagatcactggtggggtagcattgagagccccttctacgaaatctcggaaaaggctaaggaaaagaaagaacggcaagtggtctacctgagcccaaatcgggacgagtactatatgatagcggccggaaactccttgtcattgaaccaagggattcaggaagagcaggtggtcccggcacggtatcgccaggagttccttacgatcgcctgggaacagatacatgttcggagcatttttccgttccagtatttttctatcggcggttccttgattcccttcatcgaacataatgacgccaaccgcgctctcatgtcatctaatatgcagagacaagcagtccccctctcaagatcagaaaaatgcatagtgggcaccggcttggagaggcaaacagcgctcgattcgcgggtttcagtgatcgctcagcgcgagggcaaaatcatttcgtccgattcgcacaagatacttctttcctcttctggtaagacgatctcgattccattggttgcacataggcggtctaacaagaacacttgtatgcatcagaagccgagggtgcctcgcggtaagtcgattaagaaagggcagatactggcggaaggggcggcaaccgtgggtggcgaattggcactgggcaagaacgtcctcgttgcgtacatgccctgggaggggtataactttgaggacgccgtgctgatcagcgagcgcctcgtgtatgaggatatatacacaagctttcacatcaggaaatacgaaatacaaaccgacaccacgagccagggttccgctgaaaaaataacaaaacagatcccgcatctcgaagaacatcttctcaggaaccttgataggaacggagtcgtgcgcctgggtagctgggttgaaactggtgacatactggtgggaaagttgaccccccaaattgcgtcggagtcatcgtacatcgcggaagccgggttgctccgggcgatattcggactggaagtctcgacctctaaagagacaagcctcaaattgcccatcggaggacggggaagagtcattgatgtcaaatggatacaacgggatccctttgatatcatggttagggtctatatattgcaaaaaagagaaataaaggtcggagacaaagttgccggccgccatggaaataagggtataatatctaagattcttccacggcaggacatgccctacctacaggatggaacccccgtggacatggtgttcaatcctcttggcgttccctcccgcatgaacgtgggacaaattttcgagtcttctctcggtcttgccggtgacctcctcaaaaaacactataggattgcaccgttcgatgagcgctatgaacaggaggcgtccagaaagttggtcttttctgaactgtacgaggcatcaaagcagactaagaatccgtgggtctttgaaccggaatatccaggcaagtccagaattttcgatggtaggacgggtgacccgtttgagcagccagtcctcataggtaaatcatacattttgaagttgatccaccaagtggatgagaagattcacgggagatcgacagggccatactcgcttgtgacacagcaaccagttcgcgggcgggcgaagcagggcgggcagagaattggagagatggaagtttgggctcttgaggggtttggagtcgcgcacattttgcaagagatactcacatacaaaagcgatcacctgattgcccgccaagagatactcaatgcgaccatttgggggaagagggtgcctaaccacgaagacccaccagaatcgttcagggtgctggttagggaactgcgcagcctggcacttgaacttaatcacttcttggtctctgagaagaattttcaagttaacagggaggatgtttaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 22 - DNA, 662 bp, Synthetic construct
  source 1..662: mol_type = other DNA
tgggatcctagaggacgaatataggactctagagaaagactcagaggaagaatatgggagcctagagaacaaatataggactcgagagggagagggcgaatatgaaatcctagaggaagaatcagaagaagaatatgggagctctgaagatggctcagagaaggaatatgggactttagaagaagactcagaagaagactcagaggaagactcagaagacgaatatgggagcccggaggaagattctatcttaaaaaaagagggttttattgagcatcgaggaacaaaagaatttagtctaaaataccaaaaagaagtagatagattcttcttcattcttcaagaactgcatatcttgccgagatcctcatccctaaaggtacttgacaatagtattattggagtcgatacacaactcacaaaaaatacaagaagtcgactaggtggattggtccgagtgaagagaaaaaaaagccatacagaactcaaaatcttttccggagatattcattttcctgaagaggcggataagatattaggtggcagtttgataccaccagaaagagaaaaaaaagattctaaggaatcaaaaaaaaggaaaaattgggtttatgttcaacggaaaaaatttctcaaaagcaaggaaaagtattttgtttcgg

SEQ ID NO: 23 - DNA, 5753 bp, Zea mays
  source 1..5753: mol_type = genomic DNA
acgaggataaagaccctctttaacctgaattctaattgattggatatcccgcacaaggaattgaaggaagatgcgacgttttattccagggaatccccaacgaaaaatgcacactattccctcttttctatcgaatcggtcataaccactgcctacattccataaaatagtgcaccacaaataggagctaatgaataggcccgcgattccgtagaaagacatcacgaccccctgtggaaaaaaaagaatttgttgagatggaagtacagatatcatattcttaccaagataactggaagccccaaccgctaagaatcctaatgaacctagaaaaagaatacaggcccagaaaaaattacctctttttcgagaaccttttagaagttctatccatatgtgttctgatcgccaattcatattagatatactaaatccagtagcggtcaattgaaattgagagaataagctaaatgatttttactttactttttttgattctgaaatagccttcagtagctagtactcctgtgaaataattggttagatacattgactttctattcaaaaaaaattagtggatccacttaaaaaaaaaactcgctatagtcggctacgcatatgcatgcatttatgctcgtagcttatatctgcatccatagacgtttttcatttgtgtgtagaaagagctacataccctatcatattatagtacttacacaaaaaaatatctgtattatgatcctggaaatacattgagaaaatttggccccgtcgtttctagacaatcttatttttctgcacataaagaaataaggaagtcattgcaattgccggaaatactaagcctactaaaggtacgaaaatagagggtaagttaaaatctgtcatagaatatgtgtctcaattcaaatttactatttctatctattcgaaatatatcttaagattcttatgatataattaagtatatctattataaggaatattgactattgtattttttagtttacaaaataaagattttttatagatatagaataaaaaagtattctataaaataaatgaatggaattgataataagaaaattgcaaaaaaggggaactcaaaaggggaactcattaaaataaaatattttatttttcttttcccattctcatcgcctttctatccttctaatcgaacttgaaattggattcaaaagaaagctattgtgaaaataaaagaaatacctctttcagaataccctttaatttaaaaagtagaagtggaatagaatacccagttgaagggtaatgatcatacgtctgtaatgcattgtatgtcccagaataggtcccattcttctaccctttccgggtagtcgatggctattcacagctactaccttaacaccaaagaagagttcgacccaatgctttatttctgtcttagtgaatcccgattcgacattaaaagtatattgattctttcccaataaacgaagactcttttctgtaaatactgcgtatttgattccattatcgtacgataatactatattttgatttatatttgtttattgtattatacctttctatattctagatatatagaatagaagaatctcgatttgtcaagtctcatgatcgtatcaagatctatttcaattcggctcagtcttttttttttttagaaaagattgagccgaattgaattgcaatcaattagaggaataaagaagaattactgcatttcttaacttattttttctctttctattttgctttttttttattttatatggtatccatcgctttgaaaccatcaaatttgatctccttccatatttcacaagctgcggctagttcagcactccatttgcaagctgctttgataatttcattaccttcacgagcaagatcgcgcccttcgttacgagcttgtacacaggcttctaaagccacacgattagctgctgcaccaggtgcatttccccaaggatgtcctaaagttcctccaccaaattgtaatacggaatcatctccaaagatttcggtcagagctggcatatgccaaacatgaatacccccagaagccaccggtataacacctggcatggatacccagtcctgagtgaaaaagataccgcgagaacgatctttttcaataaaatcatcgcgcaataaatcaacaaaacctaaagttatttcgcgttccccttctaacttacctactactgtaccggagtggatatgatctccccccgacatacgcaatgctttagctaatacacggaaatgcataccatgatttttctgtctatcaataactgcatgcattgctcggtgaatgtgaagaagtaggccgttgtcgcggcaataatgagacaaagtagtatttgcggtgaatcctcctgttaagtagtcatgcattacaataggaacccctaattcccttgcaaatacagctctcttaatcatttcttcgcatgtacctgcagtcgcattcaagtaatgccccttgatttcaccagtttcggcttgtgctttataaattgcttcggcacaaaagacaaaacggtctctccagcgcataaatggttgtgagtttacgttttcatcatctttggtaaaatcaagtccaccgcgtagacactcataacacgctctaccgtaattttttgcggataatcccaattttggtttaatagtacatcccaataaaggacgaccgtacttgttcaacttatccctttcaacttggataccgtgaggcggaccttggaaagtttttgaataagcagggggaattcgtagatcctccaaacgtagagcgcgtaaggctttgaaaccaaatacgttacccacaatggaagtaaacatgttagtaacagaaccctcttcaaataggtctaatggataagctacataacagatatattgatctgggtccccaggaacgggctcgatgtgatagcatcgtcctttgtaacgatcaagactggtaagtccatcagtccaaacagttgtccatgtaccagtagaagattccgcagctactgcagctcctgcttcttcaggcggaaccccgagctgaggagttactcggaatgctgccaagatatcagtatccttggtttcgtactccggggtgtagtaagtcaatttataatccttaacaccagctttaaatccaacacttgctttagtttctgtttgtggtgacataagtccctccctacaactcatgaattaagaattctcacaacgacagggtctactcgatatggattaggcgtaaatgaaacctttgcaaaaatctaaaaaaaaagatatttaattaatatcaactcattaaataaaaaaaggagtatgcttaagttaatgaatatgtttcattcatatataatgtgtacaccctgtgtacgttctatcctataggaattttactataggaattcgataggaaggaattcgataggaattgagttgttgttatggtaagttaacacggttcgttattaaaccgtggatttgattcaccaaatccatcattattgtatactctttgatagatatagcgcaacccaaaccctaatctttattttacaatttttaaagttcttcccctttgatattttgaatctaaatacctaaatactaagaaaattctctgttgacagcaatctatgcttcacagtagtatatattttgtatatcgaagtcctagataggaaagtagagtaggcacagatccttcacaaaaggcgaaatttatatgaaaaaaatggattgaactttccaacggattcattccataagtaaacgattgaatgggattcgcttgggcaacgaaatcaagtgcgagtccccttttcttttttattgaattaactaattcatttccttttgacttttggatttttggatattttttttgatttggcattattcaacaagaaaaaaaatttcgacaaattcctttttttttgaaaattatgtgataattatgagaaccaatcctactacttcgcgtcccgggatttccacaattgaagaaaaaagcgtagggcgtattgatcaaattattggacccgtgctggatatcacttttcccccgggcaagttgccttatatttataatgctttgatagtcaagagtcgagacactgccgataagcaaattaatgtgacttgtgaggtacaacaattattaggaaataatcgagttagagctgtggctatgagtgctacagaggggttgatgagaggaatggaagtgattgacacgggaactcctctcagtgttccagtcggtggagctactctcggacgaatttttaacgttcttggggagcctattgacaatttgggtcctgtggatactagtgcaacatttcctattcatagatctgcgcctgcctttatcgagttagatacgaaattatctatctttgaaacaggtattaaggtggtcgatcttttagctccctatcgacgtggaggaaaaatcggactgtttgggggggcaggagtaggtaaaacagtactcatcatggaattaatcaataacattgctaaagctcatggaggcgtatccgtatttggcggagtaggggaacggactcgtgaaggaaatgatctttatatggaaatgaaggaatccggagtaattaatgaaaaaaatattgaggaatcaaaggtagctctagtctatggccaaatgaatgaaccgccgggagctcgtatgagagttggtttaactgccctaactatggcagaatatttccgagatgttaataagcaagacgtgcttttattcatcgataatatctttcgttttgttcaagcaggatcggaagtatccgccttattaggcagaatgccctccgcagtgggttatcaacctacccttagtacagaaatgggttctttgcaagaaagaattacttctaccaaaaagggatctataacttcgatccaagcagtttatgtacctgcagacgatttgaccgaccctgctcctgccacaacatttgcacatttggacgctactaccgtactttccagaggattagcttccaagggtatttatcccgcagtggatcctttagattcaacctcaactatgttacagcctcggattgttggcaacgaacattatgaaactgcgcaaagagttaaggaaactttacaacgttacaaagaacttcaggacattatcgcaattcttggattggatgaattatcggaggaggatcgtttaactgtagcaagagcacgaaaaatcgagcggttcttatcacaaccgttctttgtggcagaagtttttaccggttctccaggaaagtatgttggtcttgcagaaacaattaggggatttcaactaatcctttccggagaattagacggcctacccgaacaggctttttatttagtggggaacatcgatgaagctagcacgaaagctataaacttagaagaggagagcaaattgaagaaatgaaattaaatctttatgtactgactcctaaacgaattatttgggattgtgaagtgaaagaaatcattttatctactaatagtggccaaattggtgtattaccaaaccacgcccccattaacacagctgtagatatgggtcctttgagaatacgcctcctgaacgaccaatggttaacggcggttctgtggagtggttttgcaagaatagttaataatgagatcatcattttaggaaatgatgcagaactgggtagtgacattgatccaga

SEQ ID NO: 24 - DNA, 3977 bp, Zea mays
  source 1..3977: mol_type = genomic DNA
ttatatggtatccatcgctttgaaaccatcaaatttgatctccttccatatttcacaagctgcggctagttcagcactccatttgcaagctgctttgataatttcattaccttcacgagcaagatcgcgcccttcgttacgagcttgtacacaggcttctaaagccacacgattagctgctgcaccaggtgcatttccccaaggatgtcctaaagttcctccaccaaattgtaatacggaatcatctccaaagatttcggtcagagctggcatatgccaaacatgaatacccccagaagccaccggtataacacctggcatggatacccagtcctgagtgaaaaagataccgcgagaacgatctttttcaataaaatcatcgcgcaataaatcaacaaaacctaaagttatttcgcgttccccttctaacttacctactactgtaccggagtggatatgatctccccccgacatacgcaatgctttagctaatacacggaaatgcataccatgatttttctgtctatcaataactgcatgcattgctcggtgaatgtgaagaagtaggccgttgtcgcggcaataatgagacaaagtagtatttgcggtgaatcctcctgttaagtagtcatgcattacaataggaacccctaattcccttgcaaatacagctctcttaatcatttcttcgcatgtacctgcagtcgcattcaagtaatgccccttgatttcaccagtttcggcttgtgctttataaattgcttcggcacaaaagacaaaacggtctctccagcgcataaatggttgtgagtttacgttttcatcatctttggtaaaatcaagtccaccgcgtagacactcataacacgctctaccgtaattttttgcggataatcccaattttggtttaatagtacatcccaataaaggacgaccgtacttgttcaacttatccctttcaacttggataccgtgaggcggaccttggaaagtttttgaataagcagggggaattcgtagatcctccaaacgtagagcgcgtaaggctttgaaaccaaatacgttacccacaatggaagtaaacatgttagtaacagaaccctcttcaaataggtctaatggataagctacataacagatatattgatctgggtccccaggaacgggctcgatgtgatagcatcgtcctttgtaacgatcaagactggtaagtccatcagtccaaacagttgtccatgtaccagtagaagattccgcagctactgcagctcctgcttcttcaggcggaaccccgagctgaggagttactcggaatgctgccaagatatcagtatccttggtttcgtactccggggtgtagtaagtcaatttataatccttaacaccagctttaaatccaacacttgctttagtttctgtttgtggtgacataagtccctccctacaactcatgaattaagaattctcacaacgacagggtctactcgatatggattaggcgtaaatgaaacctttgcaaaaatctaaaaaaaaagatatttaattaatatcaactcattaaataaaaaaaggagtatgcttaagttaatgaatatgtttcattcatatataatgtgtacaccctgtgtacgttctatcctataggaattttactataggaattcgataggaaggaattcgataggaattgagttgttgttatggtaagttaacacggttcgttattaaaccgtggatttgattcaccaaatccatcattattgtatactctttgatagatatagcgcaacccaaaccctaatctttattttacaatttttaaagttcttcccctttgatattttgaatctaaatacctaaatactaagaaaattctctgttgacagcaatctatgcttcacagtagtatatattttgtatatcgaagtcctagataggaaagtagagtaggcacagatccttcacaaaaggcgaaatttatatgaaaaaaatggattgaactttccaacggattcattccataagtaaacgattgaatgggattcgcttgggcaacgaaatcaagtgcgagtccccttttcttttttattgaattaactaattcatttccttttgacttttggatttttggatattttttttgatttggcattattcaacaagaaaaaaaatttcgacaaattcctttttttttgaaaattatgtgataattatgagaaccaatcctactacttcgcgtcccgggatttccacaattgaagaaaaaagcgtagggcgtattgatcaaattattggacccgtgctggatatcacttttcccccgggcaagttgccttatatttataatgctttgatagtcaagagtcgagacactgccgataagcaaattaatgtgacttgtgaggtacaacaattattaggaaataatcgagttagagctgtggctatgagtgctacagaggggttgatgagaggaatggaagtgattgacacgggaactcctctcagtgttccagtcggtggagctactctcggacgaatttttaacgttcttggggagcctattgacaatttgggtcctgtggatactagtgcaacatttcctattcatagatctgcgcctgcctttatcgagttagatacgaaattatctatctttgaaacaggtattaaggtggtcgatcttttagctccctatcgacgtggaggaaaaatcggactgtttgggggggcaggagtaggtaaaacagtactcatcatggaattaatcaataacattgctaaagctcatggaggcgtatccgtatttggcggagtaggggaacggactcgtgaaggaaatgatctttatatggaaatgaaggaatccggagtaattaatgaaaaaaatattgaggaatcaaaggtagctctagtctatggccaaatgaatgaaccgccgggagctcgtatgagagttggtttaactgccctaactatggcagaatatttccgagatgttaataagcaagacgtgcttttattcatcgataatatctttcgttttgttcaagcaggatcggaagtatccgccttattaggcagaatgccctccgcagtgggttatcaacctacccttagtacagaaatgggttctttgcaagaaagaattacttctaccaaaaagggatctataacttcgatccaagcagtttatgtacctgcagacgatttgaccgaccctgctcctgccacaacatttgcacatttggacgctactaccgtactttccagaggattagcttccaagggtatttatcccgcagtggatcctttagattcaacctcaactatgttacagcctcggattgttggcaacgaacattatgaaactgcgcaaagagttaaggaaactttacaacgttacaaagaacttcaggacattatcgcaattcttggattggatgaattatcggaggaggatcgtttaactgtagcaagagcacgaaaaatcgagcggttcttatcacaaccgttctttgtggcagaagtttttaccggttctccaggaaagtatgttggtcttgcagaaacaattaggggatttcaactaatcctttccggagaattagacggcctacccgaacaggctttttatttagtggggaacatcgatgaagctagcacgaaagctataaacttagaagaggagagcaaattgaagaaatgaaattaaatctttatgtactgactcctaaacgaattatttgggattgtgaagtgaaagaaatcattttatctactaatagtggccaaattggtgtattaccaaaccacgcccccattaacacagctgtagatatgggtcctttgagaatacgcctcctgaacgaccaatggttaacggcggttctgtggagtggttttgcaagaatagttaataatgagatcatcattttaggaaatgatgcagaactgggtagtgacattgatccaga

SEQ ID NO: 25 - DNA, 1080 bp, Zea mays
  source 1..1080: mol_type = genomic DNA
atcttgaatcgcgaccttttggctcaactttatcctagttttgccgaaggagcaaccccttttttcaccttgaattggtccaaatatgcggaatttcttagttttcgcggaggactagatccaataaccggtggtctatggttgagcgatattgcacaccatcatttagctattgctattcttttcctgatcgctggtcatatgtataggaccaattggggtattggtcatggacttaaagatattttggaggctcataagggcccatttacaggacaaggccataaaggtctctatgaaatcctaacaacgtcatggcatgctcaattatctcttaacctagctatgctaggctctacaactattgttgtagctcatcatatgtactctatgcccccctatccatacctagctactgactacggtacacaactttccttgttcacacaccacatgtggattggcggatttctaatagttggtgctgctgcacatgcagccatttttatggtaagagactatgatccaactactcgatacaacgatctattagatcgcgtccttagacaccgcgatgcaatcatatcccaccttaactgggtatgtatatttctgggttttcacagttttggcttgtacattcataatgataccatgagtgctttaggccgtccccaagatatgttttcggataccgccatacaattacaacccatctttgctcaatggatacaaaatatccatgctggcgcgcctggcgtaacagctcctggtgcaacaacaagtaccagcttaacgtggggaggcggcgagttagtagctataggcggcaaagtagctttgttacctattccattaggaaccgcagattttttagtccatcacattcacgcatttaccatccatgtgactgtattaatacttttgaaaggtgttttatttgctcgcagttcccgtctgatacctgataaagcaaatcttggctttcgcttcccttgcgacggacctgggcgagggggaacatgtcaagtatccgcctgggatcatgttttcttaggtctattctggatgtacaattct

SEQ ID NO: 26 - DNA, 1080 bp, Sorghum bicolor
  source 1..1080: mol_type = genomic DNA
atcttgaatcgcgaccttttggctcaactttatcctagttttgccgaaggagcaaccccctttttcaccttgaattggtccaaatatgcggaatttcttagttttcgcggaggactagatccaataaccggtggtctatggttgagcgatattgcacaccatcatttagctattgctattcttttcctgatcgctggtcatatgtataggaccaattggggtattggtcatggacttaaagatattttggaggctcataagggcccatttacaggacaaggccataaaggtctctatgaaatcctaacaacgtcatggcacgctcaattatctcttaacctagctatgctaggctctacaactattgttgtagctcatcatatgtactctatgcccccctatccatacctagctactgactatggtacacaactttccttgttcacacaccacatgtggattggcggatttctaatagttggtgctgctgcacatgcagccatttttatggtaagagactatgatccaactactcgatacaacgatctattagatcgcgtccttagacaccgcgatgcaatcatatcccaccttaactgggtatgtatatttctgggttttcacagttttggcttgtacattcataatgataccatgagtgctttaggccgtccccaagatatgttttcggataccgccatacaattacaacccatctttgctcaatggatacaaaatatccatgctggcgcgcctggcgtaacagctcctggtgcaacaacaagtaccagcttaacgtggggaggcggcgagttagtagctgtaggcggcaaagtagctttgttacctattccattaggaaccgcagattttttagtccatcacattcacgcatttaccatccatgtgactgtattaatacttttgaaaggtgttttatttgctcgcagttcccgtctgatacctgataaagcaaatcttggctttcgcttcccttgcgacggacctgggcgagggggaacatgtcaagtatccgcctgggatcatgttttcttaggtctattctggatgtacaattct

SEQ ID NO: 27 - DNA, 1080 bp, Oryza sativa
  source 1..1080: mol_type = genomic DNA
atcttgaatcgcgaccttttggctcaactttatcctagtttttccgaaggagcaaccccctttttcaccttgaattggtccaaatacgcagaatttcttagttttcgcggagggctagatccaataaccggtggtctatggttgagcgatattgcgcaccatcatttagctattgctattcttttcctgatcgcaggtcatatgtataggaccaactggggtattggtcatggcctgaaagatattttggaggctcataaaggcccatttacaggacagggccataaaggtctctatgaaatcctaacaacgtcatggcatgctcaattatctcttaacctagctatgctaggctctacaaccattgttgtagctcatcatatgtactctatgcccccctatccatacctagctactgactatggtacacaactttccttgttcacacaccacatgtggattggcggatttctaatagttggtgctgctgcacatgcagccatttttatggtaagagactatgatccaacgactcgatacaacgatctattagatcgcgtccttagacaccgtgatgcaatcatatcccaccttaactgggtatgtatatttctaggttttcacagttttggtttgtacattcataatgataccatgagtgctttaggtcgtccccaagatatgttttcggataccgccatacaattacaaccgatctttgctcaatgggtacaaaatctccatgctggcgcgcctggcgtaacagctcctggtgcaacaacaagtaccagcttaacgtggggaggtggcgagttagtagcagtaggcggcaaagttgctttgttacctattccattaggaaccgcagattttttagtccatcacattcacgcatttacaatccatgtgactgtattaatacttttgaaaggtgttttatttgctcgcagttcccgtttgatacccgataaagcaaatcttggttttcgcttcccttgcgatgggcctgggcgagggggaacatgtcaagtatccgcctgggatcatgttttcttaggtctattctggatgtacaattcc

SEQ ID NO: 28 - DNA, 67 bp, Zea mays
  source 1..67: mol_type = genomic DNA
tgctttttgccggttggttccattatcacaaagccgctcccaaattggcctggttccaagatgtaga

SEQ ID NO: 29 - DNA, 67 bp, Zea mays
  source 1..67: mol_type = genomic DNA
tctacatcttggaaccaggccaatttgggagcggctttgtgataatggaaccaaccggcaaaaagca

SEQ ID NO: 30 - DNA, 64 bp, Zea mays
  source 1..64: mol_type = genomic DNA
tagctattgctattcttttcctgatcgctggtcatatgtataggaccaattggggtattggtca

SEQ ID NO: 31 - DNA, 64 bp, Zea mays
  source 1..64: mol_type = genomic DNA
tgaccaataccccaattggtcctatacatatgaccagcgatcaggaaaagaatagcaatagcta

SEQ ID NO: 32 - DNA, 64 bp, Zea mays
  source 1..64: mol_type = genomic DNA
tcatatctcatttaagttgggctagcctcttcctaggattccataccttgggcctttatgttca

SEQ ID NO: 33 - DNA, 64 bp, Zea mays
  source 1..64: mol_type = genomic DNA
tgaacataaaggcccaaggtatggaatcctaggaagaggctagcccaacttaaatgagatatga

SEQ ID NO: 34 - DNA, 69 bp, Zea mays
  source 1..69: mol_type = genomic DNA
ttggatgttcttatttggacatcttgtttgggctacaggatttatgttcttaatttcctggcgtggata

SEQ ID NO: 35 - DNA, 69 bp, Zea mays
  source 1..69: mol_type = genomic DNA
tatccacgccaggaaattaagaacataaatcctgtagcccaaacaagatgtccaaataagaacatccaa

SEQ ID NO: 36 - DNA, 67 bp, Zea mays
  source 1..67: mol_type = genomic DNA
tgatatcttggcagcattccgagtaactcctcagctcggggttccgcctgaagaagcaggagctgca

SEQ ID NO: 37 - DNA, 67 bp, Zea mays
  source 1..67: mol_type = genomic DNA
tgcagctcctgcttcttcaggcggaaccccgagctgaggagttactcggaatgctgccaagatatca

SEQ ID NO: 38 - DNA, 66 bp, Zea mays
  source 1..66: mol_type = genomic DNA
tatccatgccaggtgttataccggtggcttctgggggtattcatgtttggcatatgccagctctga

SEQ ID NO: 39 - DNA, 66 bp, Zea mays Hopscotch virus
  source 1..66: mol_type = genomic DNA
tcagagctggcatatgccaaacatgaatacccccagaagccaccggtataacacctggcatggata

SEQ ID NO: 40 - DNA, 65 bp, Zea mays
  source 1..65: mol_type = genomic DNA
gatatcttggcagcattccgagtaactcctcagctcggggttccgcctgaagaagcaggagctgc

SEQ ID NO: 41 - DNA, 65 bp, Zea mays
  source 1..65: mol_type = genomic DNA
gcagctcctgcttcttcaggcggaaccccgagctgaggagttactcggaatgctgccaagatatc

SEQ ID NO: 42 - DNA, 65 bp, Zea mays
  source 1..65: mol_type = genomic DNA
gatatcttggcagcattccgagtaactccttagctcggggttccgcctgaagaagcaggagctgc

SEQ ID NO: 43 - DNA, 65 bp, Zea mays
  source 1..65: mol_type = genomic DNA
gcagctcctgcttcttcaggcggaaccccgagctaaggagttactcggaatgctgccaagatatc

SEQ ID NO: 44 - DNA, 65 bp, Zea mays
  source 1..65: mol_type = genomic DNA
gatattcttgctgcttttagagtaactcctcagctcggggttcctccagaggaggctggtgcagc

SEQ ID NO: 45 - DNA, 65 bp, Zea mays
  source 1..65: mol_type = genomic DNA
gctgcaccagcctcctctggaggaaccccgagctgaggagttactctaaaagcagcaagaatatc

SEQ ID NO: 46 - DNA, 53 bp, Zea mays
  source 1..53: mol_type = genomic DNA
taagatttcccaggtttagccaaggcttagctcaggaccccactactcgtcgt

SEQ ID NO: 47 - DNA, 53 bp, Zea mays
  source 1..53: mol_type = genomic DNA
acgacgagtagtggggtcctgagctaagccttggctaaacctgggaaatctta

SEQ ID NO: 48 - DNA, 53 bp, Zea mays
  source 1..53: mol_type = genomic DNA
taagatttcccaggtttagccaaggcttagcttaggaccccactactcgtcgt

SEQ ID NO: 49 - DNA, 53 bp, Zea mays
  source 1..53: mol_type = genomic DNA
acgacgagtagtggggtcctaagctaagccttggctaaacctgggaaatctta

SEQ ID NO: 50 - DNA, 53 bp, Zea mays
  source 1..53: mol_type = genomic DNA
tgcgtttccctcgtttctctcaaggcttagctcaggatcctacaacaagaaga

SEQ ID NO: 51 - DNA, 53 bp, Zea mays
  source 1..53: mol_type = genomic DNA
tcttcttgttgtaggatcctgagctaagccttgagagaaacgagggaaacgca

SEQ ID NO: 52 - DNA, 69 bp, Zea mays
  source 1..69: mol_type = genomic DNA
gatcggttttttttcattcttcaagaactgcatatcttgccgagatcctcatccctaaaggtacttgac

SEQ ID NO: 53 - DNA, 69 bp, Zea mays
  source 1..69: mol_type = genomic DNA
gtcaagtacctttagggatgaggatctcggcaagatatgcagttcttgaagaatgaaaaaaaaccgatc

SEQ ID NO: 54 - DNA, 69 bp, Zea mays
  source 1..69: mol_type = genomic DNA
gatcggtttttttttcattcttcaagaactgcatatcttgccgagatcctcatccctaaaggtacttga

SEQ ID NO: 55 - DNA, 69 bp, Zea mays
  source 1..69: mol_type = genomic DNA
tcaagtacctttagggatgaggatctcggcaagatatgcagttcttgaagaatgaaaaaaaaaccgatc

SEQ ID NO: 56 - DNA, 69 bp, Zea mays
  source 1..69: mol_type = genomic DNA
gatagattcttcttcattcttcaagaactgcatatcttgccgagatcctcatccctaaaggtacttgac

SEQ ID NO: 57 - DNA, 69 bp, Zea mays
  source 1..69: mol_type = genomic DNA
gtcaagtacctttagggatgaggatctcggcaagatatgcagttcttgaagaatgaagaagaatctatc

SEQ ID NO: 58 - protein, 455 aa, synthetic construct
MIEFPEYRDSSAAPKISDLERLMDRSLTNSQYVDGANAARLLGTFIRSFRSVIGSAEESATRANLVEAHDEAKLFGLMLSAGFDLICNAEYVHGRLVNNKWIYCHRGGEPAVAYYSFLKQCPRCCLDRGLEGRLSGAQHKPTSHHIGEITTVAIALLLQLVAAANENPFEIATITKQSHDVDAIGFRDDLLVLFEIKASPMVSFPLVTELEEPMLQEGPDGPVEYRQHSLVDLTLQGREFAVAIPHAETAIPLGEREGESWPYEPLIDYFSVPANAASYLQAWIELYAAYRTPKTQRAGRTAALAYLVNGWGDEIDSNKTKPGLGRTDDVKKGTYQLLKFGSYYRDDAASVPVRGALVANLDPLFLRPGYIDGLSDVRWGHGRDFTLEEGEYRIAEGSLRHLYDAILAFNDPLLNDPLLQEIFDLGAVERKLANGDLEALLDKWIARPEIVLDPS

SEQ ID NO: 59 - DNA, 1626 bp, synthetic construct
  source 1..1626: mol_type = other DNA
atggcgcaggtctcccgcatttgtaatggtgtgcagaatccttcgctcatcagcaatttgtccaaaagctcccaaagaaagtcgcctctcagcgtttcattgaaaacgcagcagcatccccgcgcctacccgatttcttcttcatggggacttaaaaagtcgggcatgacactcataggctccgaacttcggccactcaaagtgatgtcttccgtttcaacggcgtgtatgctgatgattgaatttccagagtatagggattcatctgcggcgccgaaaataagcgaccttgaaaggctgatggataggtcactcactaattcgcagtacgtggatggagccaatgctgcgcgcctgttgggcacttttattcggtcgtttcgctctgttatcgggtctgcagaagagtcagctaccagggcgaatctcgtggaggcacacgacgaggctaaactgtttggcctgatgttgtctgcagggttcgacctcatttgcaatgccgaatatgttcatggacgcctggtcaacaacaaatggatatactgccacagaggcggcgaaccagcagtcgcctactattcattcctgaagcaatgtccgcggtgttgtctcgatagaggtttggaaggaagactgtccggcgcacaacataaaccaacatctcatcacattggtgaaatcactacagtggcaattgctctcctgttgcagctggttgccgcggcaaacgaaaaccctttcgaaatagcgacgataacgaagcaatcgcacgacgtcgacgcaatcggatttagagatgaccttttggttctctttgaaatcaaggcttctccgatggttagctttcctcttgttacggaacttgaagagcccatgctccaagaggggcctgatggccctgtcgaatatcggcaacattccctggtcgacctcacgctccaaggacgcgagttcgccgtcgctataccgcacgcggagacggcaatcccgcttggagaaagggagggagaaagctggccgtacgaaccgctcatcgactatttttcggttccagctaatgctgcctcatacctccaggcttggatcgagctttacgctgcgtaccggacccccaaaactcaacgcgcaggtcggacggccgctcttgcctaccttgtgaacgggtggggtgacgagatcgattcaaacaaaacgaagcccggactcggcagaacggatgatgtcaagaagggaacatatcagctgcttaaatttggaagctactaccgcgatgatgccgcttcagtgccggtgaggggggcactcgtggctaacttggacccgctttttcttcggcccggttacatagatgggctttctgatgtgagatgggggcacggcagagacttcaccctggaagagggagaatataggattgccgaaggctctctgcggcacttgtatgatgcaatcttggcttttaatgatccactcctcaacgatccgttgctccaagaaatattcgatctcggcgcagttgaacggaaattggcaaacggcgacctcgaggcgctgctcgataagtggattgcgcgccctgagattgttctggacccatccccttatgatgttcctgattatgcttaa

SEQ ID NO: 60 - protein, 533 aa, synthetic construct
MAQVSRICNGVQNPSLISNLSKSSQRKSPLSVSLKTQQHPRAYPISSSWGLKKSGMTLIGSELRPLKVMSSVSTACMLMIEFPEYRDSSAAPKISDLERLMDRSLTNSQYVDGANAARLLGTFIRSFRSVIGSAEESATRANLVEAHDEAKLFGLMLSAGFDLICNAEYVHGRLVNNKWIYCHRGGEPAVAYYSFLKQCPRCCLDRGLEGRLSGAQHKPTSHHIGEITTVAIALLLQLVAAANENPFEIATITKQSHDVDAIGFRDDLLVLFEIKASPMVSFPLVTELEEPMLQEGPDGPVEYRQHSLVDLTLQGREFAVAIPHAETAIPLGEREGESWPYEPLIDYFSVPANAASYLQAWIELYAAYRTPKTQRAGRTAALAYLVNGWGDEIDSNKTKPGLGRTDDVKKGTYQLLKFGSYYRDDAASVPVRGALVANLDPLFLRPGYIDGLSDVRWGHGRDFTLEEGEYRIAEGSLRHLYDAILAFNDPLLNDPLLQEIFDLGAVERKLANGDLEALLDKWIARPEIVLDPS

SEQ ID NO: 61 - DNA, 59 bp, Zea mays
  source 1..59: mol_type = genomic DNA
tattggaagattaatcgaccaaaataaccgtgagcagccacaatattataagtctcttc

SEQ ID NO: 62 - DNA, 59 bp, Oryza sativa
  source 1..59: mol_type = genomic DNA
tattggaagattaatcggccaaaataaccatgagcggccacaatattataagtttcttc

SEQ ID NO: 63 - DNA, 60 bp, Sorghum bicolor
  source 1..60: mol_type = genomic DNA
atattggaagattaatcgaccgaaataaccgtgagcagccacaatattataagtctcttc

(19) United States
(12) Patent Application Publication
(10) Pub. No.: US 2026/0132412 A1
(43) Pub. Date: May 14, 2026

(54) PLASTID TRANSFORMATION BY COMPLEMENTATION OF PLASTID MUTATIONS

(71) Applicant: Plastomics Inc., St. Louis, MO (US)

(72) Inventor: Jeffrey Staub, Wildwood, MO (US)

(21) Appl. No.: 19/311,616

(22) Filed: Aug. 27, 2025

Related U.S. Application Data
(62) Division of application No. 18/180,399, filed on Mar. 8, 2023.
Application No. 18/180,399 is a continuation of application No. PCT/US2021/048399, filed on Aug. 31, 2021.
(60) Provisional application No. 62/706,760, filed on Sep. 9, 2020.
(60) Provisional application No. 63/180,766, filed on Apr. 28, 2021.

(51) Int. Cl. C12N 15/82; C12N 9/22

(52) U.S. Cl. CPC C12N 15/8214; C12N 9/22; C12N 15/8213; C12N 15/8269; C12N 2310/20

(57) ABSTRACT

A method of expressing an agronomically or non-agronomically beneficial trait in a plant plastid comprising expressing an exogenous nucleic acid in the plant to produce non-photosynthetic mutant plants, and using callus grown from the mutant plants as recipients for introduction of a construct having a functional copy of the mutated gene and a gene conferring an agronomically or non-agronomically beneficial trait. Embodiments provide for mutations in chloroplast-encoded genes, as well as mutations in nuclear-encoded genes targeted to the chloroplast that are required for photosynthesis, plants and plant parts produced from such methods, as well as kits for performing the methods as described.

### CROSS-REFERENCE TO RELATED APPLICATIONS

This application is a divisional of U.S. patent application Ser. No. 18/180,399, filed Mar. 8, 2023, which is a bypass continuation of International Application No. PCT/US2021/048399, filed on Aug. 31, 2021, and published as WO2022/055750A1 on Mar. 17, 2022, and which claims priority to and the benefit of U.S. Provisional Application Ser. No. 62/706,760 filed on Sep. 9, 2020 and Ser. No. 63/180,766 filed on Apr. 28, 2021, the contents of which are hereby incorporated in their entireties.

### INCORPORATION OF SEQUENCE LISTING

The sequence listing that is contained in the file named “2025-08-27_Sequence_Listing_P1810001US2.xml,” which is 125,864 bytes as measured in Microsoft Windows operating system and was created on Aug. 27, 2025, and is filed electronically herewith and incorporated herein by reference.

### FIELD OF THE DISCLOSURE

The disclosure relates to plant genetics. More particularly, the disclosure relates to methods for generating non-photosynthetic plant mutants and transforming mutant plant plastids by complementation of the non-photosynthetic defect or mutation.

### BACKGROUND

A plastid is a class of plant subcellular organelles that have evolved different functions depending on the tissue in which they arise. Chloroplasts are the best-known plastid type, and are specialized for chlorophyll production and active photosynthesis in green leaf cells. Other plastid types include root amyloplasts specialized for starch accumulation, chromoplasts in flower petals that are specialized for colored pigment development, and proplastids that are undeveloped precursors to other plastid types and reside in dark-grown embryogenic tissues often used in cell culture systems.

The plastid carries its own genome, which is a double-stranded circular DNA (dsDNA) of ˜155 kilobases encoding ˜110 genes. The plastid is a polyploid genetic system, and the number of plastids and plastid genomes differs depending on which cell type they reside in. For example, in leaf cells, chloroplasts and their DNA are abundant with up to ˜100 chloroplasts per cell and ˜100 dsDNAs per chloroplast, for a total of ˜10,000 DNAs per leaf cell.

Most of the genes encoded by the plastid genome are required for maintenance of the organelle itself (ribosomal RNAs, tRNAs, ribosomal proteins, etc.). In addition, ˜35 plastid-encoded genes encode components of photosynthesis, with the large subunit gene (rbcL) of RUBISCO being the most well-studied. However, the vast majority of proteins that reside in the organelle are nuclear-encoded and imported into the organelle. Nuclear-encoded proteins include structural proteins, enzymes, and transcription/translation factors that help control expression of plastid-encoded genes.

The insertion of transgenes into the plastid genome was first achieved in tobacco in the 1990's. Success in plastid transformation of tobacco was facilitated by its routine tissue culture and transformation system that utilizes green leaf tissue with abundant chloroplasts as a recipient for transforming DNA. The favored selectable marker used in nearly all plastid transformation experiments is a bacterial-derived aadA gene that confers resistance to the antibiotics spectinomycin and streptomycin. Under antibiotic selection, sensitive green leaf cells will bleach and their growth will be inhibited (due to lack of plastid gene translation due to the antibiotic). Only transformed plastids that receive the selectable aadA marker gene will acquire resistance to the antibiotics and remain green and continue to grow normally. Plastid transformation has also been reported in the photosynthetic green algae, Chlamydomonas reinhardtii, which was facilitated by the availability of mutant strains that are non-photosynthetic due to deletion in one or more plastid-encoded genes required for photosynthesis. Deletion mutants were first created by treatment of cells with 5-fluoro-deoxy-uridine, a nucleotide base analog that causes disruption of plastid DNA replication and subsequent generation of deletion mutants.

Chlamydomonas is a genus of unicellular green algae. Numerous recipient Chlamydomonas strains with deletions in a number of photosynthesis genes have been used successfully to select plastid transformants in certain Chlamydomonas species. Transgenes can be easily inserted into the plastid genome alongside the complementing DNA in certain Chlamydomonas species (reviewed in Day and Goldschmidt-Clermont, 2011). In rare cases, complex plastid DNA rearrangements can occur during another culture of some grass species, such as barley, and mutant regenerated plantlets have been recovered. However, these plants are typically albino and do not survive. In other cases, some parasitic plants that have a symbiotic relationship with other plants have lost large portions of their plastid genome during evolution and are non-photosynthetic. However, these plants rely on the host plant for survival. In still some other cases, the chemical mutagen nitrosomethylurea (NMU) has been used to generate plastome mutations in sunflower, tomato, pepper and nicotiana (reviewed in Prina, Pacheco and Landau 2012). However, plastid mutations induced by NMU are typically single nucleotide transversion and thus can undergo spontaneous reversion to wild-type (for example, Usatov et al, 2004).

In some cases, plastid transformation has been used to mutate or delete a plastid gene to study the function of that gene. In one example, the native tobacco chloroplast-encoded rbcL gene, encoding the large subunit of RUBISCO, was replaced with the rbcL gene from an algal species (Whitney et al., 2008). The algal rbcL gene was nearly non-functional in tobacco, resulting in a non-photosynthetic tobacco line that required high CO₂ for growth. In subsequent experiments, the non-photosynthetic tobacco line was used as a recipient for new variants of rbcL genes derived from other species, in an attempt to identify better RUBISCO variants. Others have also used this strategy in tobacco to create deletion mutants in other photosynthetic genes. For example, Klaus et al., (2003) used tobacco chloroplast transformation and selection for spectinomycin resistance to first insert an aadA selectable marker into the coding region of a chloroplast photosynthesis related gene to create a non-photosynthetic mutant. Subsequently, the chloroplast deletion mutant lines were used for retransformation by selection for kanamycin resistance conferred by an aphA-6 selectable marker gene while a wild-type copy of the mutated photosynthesis gene was introduced at the same time was used to restore photosynthesis. Such strategies require a working plastid transformation system including an efficient antibiotic selectable marker to first create the deletion mutant line and for subsequent selection of retransformed chloroplasts and do not apply to new plant species where plastid transformation does not exist.

In an effort to study double-strand DNA breakage and repair in chloroplasts, Kwon et al. (2010) utilized a Chlamydomonas-derived I-CreII homing endonuclease enzyme that recognizes a conserved ˜30 bp enzyme recognition site fortuitously present in the coding region of the plastid-encoded psbA gene, encoding the D1 protein of Photosystem II that is required for photosynthesis. The authors created Arabidopsis nuclear transgenic lines with an inducible transgene for the I-CreII enzyme, fused to a chloroplast transit peptide. Upon induced expression, the enzyme is targeted to chloroplasts where it cuts the plastid genome at its recognition site within the psbA gene. Spontaneous re-ligation of the Arabidopsis plastid genome occurred in some cases in an imperfect fashion such that deletions in the psbA gene were created, resulting in non-photosynthetic (albino) mutant sectors in leaves. This approach is limited to deletion to the psbA locus and no attempt was made to purify albino sectors or mutant lines to homoplasmy to be used as a recipient for subsequent chloroplast transformation in Arabidopsis. Hajdukiewicz et al., (2001) showed that recombination can occur between an endogenous chloroplast sequence consisting of multiple copies of a 5 base pair AT-rich directly repeated sequence and a transplastomic lox site during a plastid transformation experiment, that resulted in deletion of the intervening sequences. The 5 bp repeated sequence motif was located ˜500 bp away from the lox site. Corneille et al. (2003) identified a second case of recombination between lox sites and a region in the chloroplast psbA gene promoter, resulting in a 17.3 kb deletion in the chloroplast genome. In the example of Kwon et al. (2010), the deletion endpoints mapped to the I-CreII site and 6-16 bp perfect or imperfect direct repeated sequences.

The nuclear expression of engineered constructs encoding mitochondria-targeted and site-specific DNA nucleases has been used to selectively eliminate mutant mtDNA sequences in heteroplasmic cells and shift mtDNA ratios back to wild-type levels (reviewed in Patananan et. al., 2016).

A recent study in rice and rapeseed indicates that MitoTALENS may also be active in plants (Kazama et al., 2019). Kazama et al. used mitoTALENs in an attempt to delete the mitochondrial encoded orfs that are responsible for cytoplasmic male sterility in a specific variety of rice and rapeseed. These authors indicate that unexpected large deletions and changes in the configuration of the mitochondrial genome are obstacles to mitoTALEN applications in plants (Kazama et. al, 2019).

Short direct or inverted repeated sequences have been previously implicated in chloroplast genome rearrangements (see Kim and Lee, 2005). Likewise, has been shown that duplication of transgene expression sequences located on transforming DNA constructs during plastid transformation can result in deletion of intervening sequences.

### SUMMARY OF THE DISCLOSURE

A platform for precision engineering of agronomically and/or non-agronomically beneficial traits into a plant chloroplast using plastid transformation via complementation of a non-photosynthetic defect or mutation is provided. An objective of the present disclosure is to provide methods for transforming plant chloroplasts by complementation of plastid mutants. Another objective of the present disclosure is to provide plant cells containing plastid genomes comprising deletion and other mutations in plastid genes.

In certain embodiments, a method of expressing an agronomically and/or non-agronomically beneficial trait in a plant plastid comprising: (a) expressing an exogenous nucleic acid in the plant nucleus, wherein the exogenous nucleic acid is operably linked to a promoter functional in plants and a chloroplast transit peptide; fused to a protein such as a TALEN, Zinc Finger, or restriction endonuclease capable of creating a double-strand break and subsequent mutation in the chloroplast genome (b) selecting a recipient mutant plant, embryo or callus having non-photosynthetic chloroplasts (c) growing a callus of the selected recipient mutant plant in culture; (d) transforming the mutant plant-line callus with a plastid transformation vector comprising a wild-type copy of the mutated chloroplast gene and one or more agronomically and/or non-agronomically beneficial trait genes; and (e) selecting green, photosynthetic callus; and regenerating a green plant carrying photosynthetic chloroplasts and the agronomically and/or non-agronomically beneficial trait, wherein the recipient mutant plant is a non-photosynthetic homoplasmic chloroplast mutant plant line. In some embodiments, the callus is grown in culture in dark or light conditions. In another embodiment, the promoter is a seed-specific promoter or an embryo-specific promoter. In another embodiment, the plastid transformation vector comprises a chloroplast transformation vector. In another embodiment, the plant comprises a corn plant or a soy plant. In another embodiment, the non-photosynthetic homoplasmic chloroplast mutant plant line comprises a mutation in a chloroplast gene. In another embodiment, the mutation in a chloroplast gene confers a non-green phenotype when grown under light conditions. In another embodiment, the disclosure provides a chloroplast-transformed plant generated by such a method. In another embodiment, the disclosure provides a plant part of such a plant, selected from the group consisting of a seed, embryo, stem, callus, meristem, leaf, and root. In another embodiment, the disclosure provides a seed produced by such a plant.

In another aspect, the disclosure provides a method for modification of one or more chloroplast genes in a plant comprising expressing in a plant nucleus an exogenous nucleic acid linked to a chloroplast transit peptide. In one embodiment, expression of the exogenous nucleic acid, which encodes a protein capable of creating a double-strand break in chloroplasts such as a TALEN or Zinc Finger endonuclease, results in a deletion mutant plant having at least one non-photosynthetic plastid. In another embodiment, the method further comprises transforming the at least one non-photosynthetic plastid with an expression construct comprising a wild-type or functional copy of the mutant gene. In another embodiment, the disclosure provides a non-photosynthetic chloroplast produced by such a method.

In another aspect, the disclosure provides a kit comprising: a single-use container comprising a callus or seed produced from a plant part as described herein. In one embodiment, the kit further comprises reagents for transformation, cell culture, or both.

In another aspect, the disclosure provides transplastomic plant cells comprising plastids containing one or more heterologous DNA insertion(s) into the genome of the plastids, wherein a selectable antibiotic resistance-or herbicide resistance-conferring gene is absent from the genome of the plastids, wherein the plant cell is not a tobacco plant cell, and wherein DNA sequence insertion, deletions, and/or substitutions resulting from selectable antibiotic resistance-or herbicide resistance-conferring gene excision are absent from the genome of the plastids are provided. Also provided are whole plants, plant parts (e.g., seeds, leaves, tubers, roots, stems, or flowers), as well as callus and/or embryogenic tissue containing the aforementioned transplastomic plant cells, the use of the plants, plant cells, and tissues in agriculture, and methods of making the plant cells, plants, plant parts, and tissues.

In another aspect, the disclosure provides transgenic plant cells comprising: (i) plastids containing a plastid genome comprising a loss-of-function mutation in at least one plastid photosynthetic gene; and, (ii) an insertion of a transgene in the nuclear genome of the plant cell, wherein the transgene encodes a product which complements the loss-of function mutation and wherein the plant cell is photosynthetic are provided. Also provided are whole plants, plant parts (e.g., seeds, leaves, tubers, roots, stems, or flowers), as well as callus and/or embryogenic tissue containing the aforementioned transgenic plant cells, the use of the plants, plant cells, and tissues in agriculture, and methods of making the plant cells, plants, plant parts, and tissues.

In another aspect, the disclosure provides methods for transforming a plant plastid with a first DNA molecule comprising: (a) introducing at least one DNA molecule comprising a first plastid gene DNA sequence into a recipient homoplasmic non-photosynthetic plant cell comprising plastids with a mutation in the first plastid photosynthetic gene DNA sequence to obtain a transformed plant cell containing the DNA molecule comprising the plastid photosynthetic gene DNA sequence; (b) exposing the transformed plant cell from step (a) to light sufficient to support greening of a photosynthetic plant cell; and, (c) selecting a green photosynthetic plant cell comprising a transformed plant plastid containing a plastid genome comprising the wild-type plastid photosynthetic gene DNA sequence from the plant cells exposed to the light in step (b) are provided.

In another aspect, the disclosure provides plant cells comprising plastids containing a plastid genome comprising a loss-of-function mutation in at least one plastid photosynthetic gene, wherein the plant cell is non-photosynthetic, is homoplasmic, and is not a tobacco or Arabidopsis plant cell are provided. Also provided are whole plants, seedlings, plant parts (e.g., seeds, leaves, tubers, roots, stems, or flowers), as well as callus and/or embryogenic tissue containing the aforementioned plant cells, the use of the plants, plant cells, and tissues in various methods including methods of transformation, and methods of making the plant cells, plants, plant parts, and tissues. In the context of certain methods provided herein, such cells are referred to as recipient homoplasmic non-photosynthetic plant cells.

In another aspect, the disclosure provides methods of making a non-photosynthetic plant cell comprising obtaining a plant wherein at least one enzyme capable of creating one or more double-stranded breaks or a nucleotide substitution mutation in a plastid genome is provided in a plastid of the plant; and, selecting a plant cell line comprising plastids containing a plastid genome comprising a loss-of-function mutation in at least one plastid photosynthetic gene, wherein the plant cell is non-photosynthetic, is homoplasmic, and is not a tobacco or Arabidopsis plant cell. In certain embodiments of the methods, the enzyme or enzymes comprise a Zinc finger nuclease, a TALEN, a meganuclease, a restriction endonuclease, or a TALE-base editor wherein the TALE-base editor is TALE-cytosine deaminase or TALE-deoxyadenine deaminase.

In another aspect, the disclosure provides methods for transforming a plant with a heterologous DNA molecule comprising: (a) introducing a heterologous DNA molecule into (i) a recipient homoplasmic non-photosynthetic plant cell comprising plastids with a mutation in the plastid photosynthetic gene DNA sequence to obtain a transformed plant cell containing the heterologous DNA molecule, wherein the heterologous DNA molecule comprises a promoter, and DNA encoding a chloroplast transit peptide (CTP) and a protein having an enzymatic and/or biological activity of a wild-type protein encoded by the wild-type plastid photosynthetic gene DNA sequence, wherein the promoter, DNA encoding the CTP and protein are operably linked; (b) exposing the transformed plant cell from step (a) to light sufficient to support greening of a photosynthetic plant cell; and, (c) selecting or screening for a green photosynthetic plant cell comprising a transformed plant containing a nuclear genome comprising the heterologous DNA molecule from the plant cells exposed to the light in step (b) are provided. In certain embodiments of the methods, the recipient homoplasmic non-photosynthetic plant cells include dicot or monocot plant cells.

### BRIEF DESCRIPTION OF THE DRAWINGS

FIG. 1 shows gene expression signals that are used to drive expression of the selectable marker gene and a GFP visual marker to help monitor plastid transformation at an early stage.

FIG. 2 shows an example of the deletion mutant and complementation. In this case, the trait gene(s) of interest is cloned adjacent to the complementing plastid fragment into a nearby intergenic region.

FIG. 3 shows a flowchart demonstrating a protocol for restoration of photosynthesis.

FIG. 4 shows a flowchart demonstrating a protocol for transformation of soybean chloroplasts.

FIG. 5 demonstrates that agronomically and/or non-agronomically important gene can be targeted to the region between the two disrupted genes such that complementation of both genes ensures the integration of the agronomic and/or non-agronomic gene between them.

FIG. 6 shows a 5.7 kb maize chloroplast genome region (SEQ ID 23) surrounding the rbcL gene coding region (SEQ ID 24) that has an abundantly repeated AT-rich sequence.

FIG. 7 is examples of multiple repeated sequences in the maize rbcL genome region.

FIG. 8 shows a psaA/B region of the maize chloroplast genome targeted by TALENs, candidate TALEN cutting sites and locations of surrounding sequence microhomologies. Examples of direct repeat sequences (DR1 through DR4) are shown. Note that DR1 and DR3 and adjacent to each other in the genome and make up a larger imperfect direct repeat (DR1-DR3).

FIG. 9 shows a map of direct repeats in chloroplast psaA-psaB Region.

FIG. 10 shows a nuclear transformation vector pPTS150 which encodes a chloroplast-targeted AscI restriction enzyme

FIG. 11 shows complementation of chloroplast non-photosynthetic lines resulting in a diagnostic new restriction endonuclease site or no site at all.

FIG. 12 includes images of independent T0 transgenic lines that carry that chloroplast targeted AscI restriction enzyme that were observed to have yellow or albino leaf sectors.

FIGS. 13A and 13B illustrate representative examples of sequence conservation of chloroplast psaA photosynthetic genes from maize (SEQ ID 25), sorghum (SEQ ID 26) and rice (SEQ ID 27) and resulting identity of the TALEN or restriction enzyme-mediated double-stranded break targeting sequences across species.

FIG. 14 shows an Agrobacterium T-DNA vector carrying a pair of chloroTALEN cassettes between the Left and Right Border elements required for transfer into the nuclear genome of plants.

FIG. 15 shows an A188 chloroplast genome chloroTALEN target sequences and coordinates in the psaA 1-L (SEQ ID 28) and psaA 1-R (SEQ ID 29) sites, psaA 2-L (SEQ ID 30) and psaA 2-R (SEQ ID 31) sites, psaB 1-L (SEQ ID 32) and psaB 1-R (SEQ ID 33) sites, psaB 2-L (SEQ ID 34) and psaB 2-R (SEQ ID 35) sites, rbcL 1-L (SEQ ID 36) and rbcL 1-R (SEQ ID 37) sites, and rbcL 2-L (SEQ ID 38) and rbcL 2-R (SEQ ID 39) sites

FIG. 16 is images TO plants derived from transformation with a chloroTALEN construct that targets the psaB gene where albino leaf sectors were observed on the TO plants.

FIG. 17A sequences show wild-type rbcL gene sequence (SEQ ID 40) and its reverse complement (SEQ ID 41) prior to and mutant rbcL gene sequence (SEQ ID 42) and its reverse complement (SEQ ID 43) after conversion of C to T at rbcL amino acid 52 converts a glutamine codon to a stop codon (Q52*).

FIG. 17B shows the chimeric TALE-cytosine deaminase genes expressed from either the maize or rice ubiquitin promoter, the nos terminator and are targeted to chloroplasts via the Rubisco small subunit transit peptide (CTP).

FIG. 17C shows 5 chloroplast C [symbol] T mutations were observed in the PTS424-Bar-67 lines, including 4 mutations in the rbcL gene and 1 mutation in the adjacent atpB gene.

FIG. 18A shows independent plastid non-photosynthetic mutant lines PTS424-Bar-9b, PTS424-Bar-40a and PTS424-Bar-67 each identified in tissue culture after transformation with the chloroTALE-DddA-UGI construct, all with a similar strong pigment deficient phenotype resulting from single nucleotide mutations in the rbcL and atpB plastid genes. The plants were regenerated from independent bialaphos resistant callus events, that each originally had a mixture of green plants, and completely bleached yellow-colored plants (arrows in FIG. 18A).

FIG. 18B shows that after transformation with the chloroTALE-DddA-UGI construct, selection on bialaphos-containing media and subsequent plant regeneration in the light, 1 pale green pigment mutant plant line (arrow on left) was observed termed PTS419-Bar-12 and several regenerated plants that were mixed green and pale green (arrow on right), which all had single nucleotide mutations in the plastid psaA and psaB genes.

FIG. 18C is a unique albino regenerated plant, termed rpoB TALEN, observed after transformation with the TALEN construct, and had a single nucleotide insertion in the plastid rpoB gene.

FIGS. 19A-19B show chloroplast transformation vectors PTS442 and PTS443 where complementing rbcL and atpB gene sequences carry the wild-type allele at each of the 5 C [symbol] T mutation sites, to restore full photosynthetic ability to complemented lines are shown in FIG. 19A and chloroTALE-DddA-UGI target sites in the rbcL coding region (SEQ ID 44) and its reverse complement (SEQ ID 45) have been mutated at several positions as shown in FIG. 19B to prevent further mutation in the complemented lines.

FIGS. 20A-20B illustrate the wild-type psaB (SEQ ID 46) TALEN target site and its reverse complement (SEQ ID 47) intended chloroplast mutation of glutamine codon at psaB amino acid 14 (Q14*) (SEQ ID 48) and its reverse complement (SEQ ID 49) as shown in FIG. 20A and the observed mutations in the psaA and psaB plastid genes after transformation with the chloroTALE-DddA-UGI construct in FIG. 20B.

FIGS. 21A-21B illustrate the PTS439 and PTS440 constructs in FIG. 21A that complement the psaA/psaB mutations, insert transgenes of interest into the intergenic region between the plastid psaA and psaB genes, and the chloroTALE-DddA-UGI target sites in the psaB coding region (SEQ ID 50) and reverse complement (SEQ ID 51) that have been mutated at several positions as shown in FIG. 21B to prevent further mutation in the complemented lines.

FIG. 22 shows the wild type rpoB gene fragment (SEQ ID 52) and reverse complement (SEQ ID 53), the single T nucleotide insertion in the plastid rpoB gene (SEQ ID 54) and reverse complement (SEQ ID 55, and complementing chloroplast DNA that removes the added T nucleotide and creates new silent mutations (SEQ ID 56) and reverse complement (SEQ ID 57) in the complemented lines that facilitate easy identification.

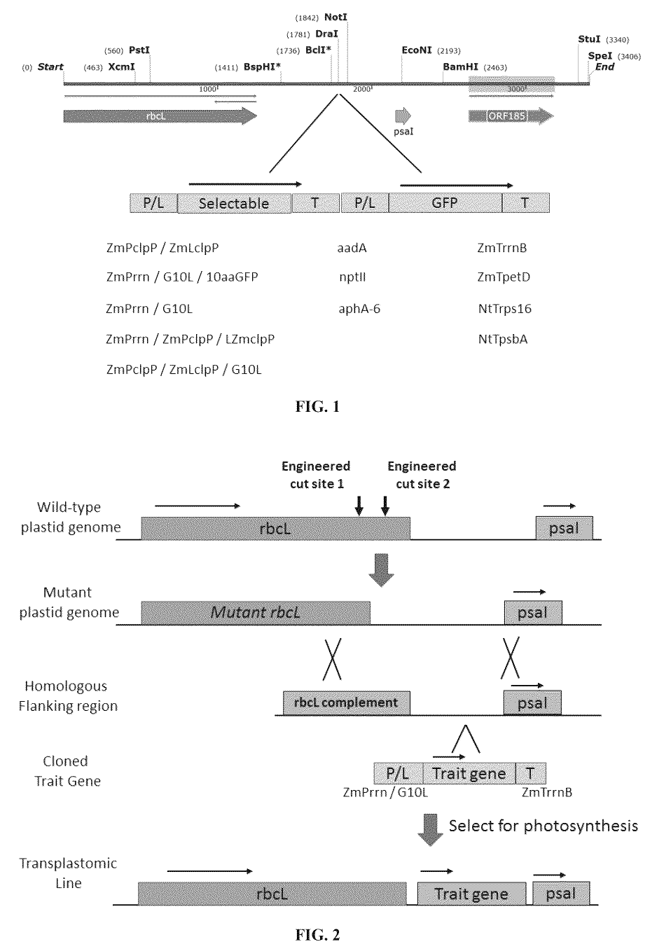

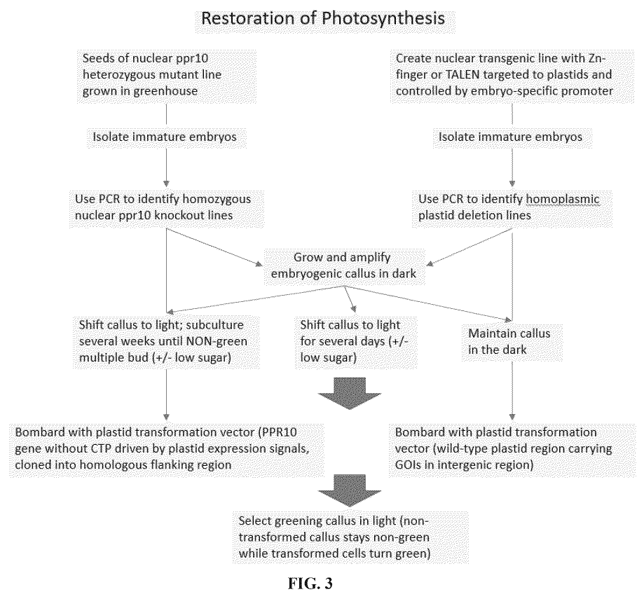

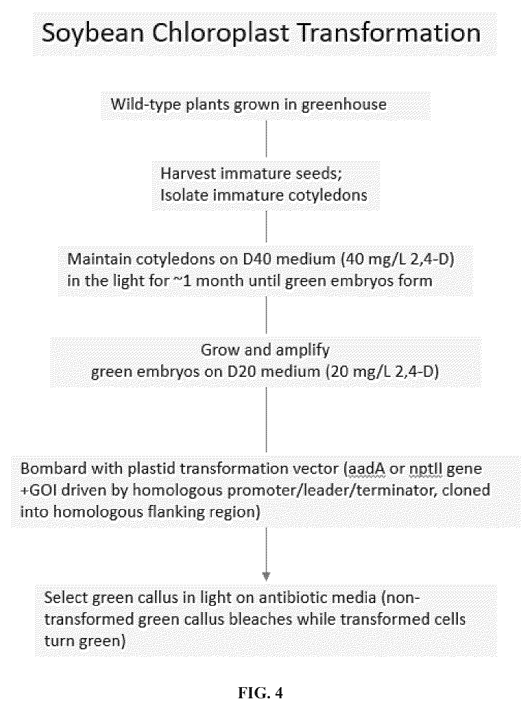

### BRIEF DESCRIPTION OF SEQUENCES

SEQ ID NO:1—Maize plastid ClpP promoter and ClpP leader (PclpP-LclpP).

SEQ ID NO:2—Maize plastid ClpP promoter-ClpP leader-Phage T7 gene 10 ribosome

binding site.

SEQ ID NO:3—Maize Prrn promoter G10L+10 amino acids of GFP.

SEQ ID NO:4—Maize Prrn 16s rDNA promoter-maize ClpP promoter-ZmClpP leader.

SEQ ID NO:5—Maize petD gene terminator ZmTpetD.

SEQ ID NO:6—Tobacco rps16 gene terminator Trps16.

SEQ ID NO:7—Tobacco psba gene terminator TpsbA (short).

SEQ ID NO:8—E. coli terminator from rrnB gene (Ecoli TrrnB).

SEQ ID NO:9—FspI amino acid sequence (CTP2 transit peptide not shown).

SEQ ID NO:10—Maize codon optimized FspI coding region (CTP2 transit peptide not shown).

SEQ ID NO:11—Maize codon-optimized amino acid sequence (derived from Chlamydomonas reinhardtii).

SEQ ID NO:12—Maize codon-optimized Nucleotide sequence.

SEQ ID NO:13—PTS424 rbcL TALE, TALE-DddA-UGI targeting left rbcL TALE binding site.

SEQ ID NO:14—PTS424 rbcL TALE, TALE-DddA-UGI targeting right rbcL TALE binding site.

SEQ ID NO:15—PTS438 CTP-RBCL, chimeric gene expressed from maize ubiquitin promoter-targeted chloroplast via CTP.

SEQ ID NO:16—PTS444 CTP-ATPB, chimeric gene expressed from rice ubiquitin promoter —targeted chloroplast via CTP.

SEQ ID NO:17—PTS419 PSAB LEFT, TALE repeat DNA sequences cloned into chimeric genes carrying TALE-DddA-UGI scaffolds.

SEQ ID NO:18—PTS419 PSAB RIGHT, TALE repeat DNA sequences cloned into chimeric genes carrying TALE-DddA-UGI scaffolds.

SEQ ID NO:19—PTS437 CTP-PSAB, maize ubiquitin 1 promoter sequence.

SEQ ID NO:20—PTS445 CTP-PSAA, maize nos terminator sequence.

SEQ ID NO:21—PTS446 CTP-RPOB, amino acid coding region of rpoB codon optimized, synthesized and cloned with N-terminal fusion to maize EPSPS CTP.

SEQ ID NO:22—PTS447 RPOB COMPLEMENTING DNA FRAGMENT-plastid PTS447 carrying a 662 bp fragment of the rpoB gene surrounding the T insertion mutation sequence.

### DETAILED DESCRIPTION

Embodiments of the present disclosure provide methods of expressing an agronomically and/or non-agronomically beneficial trait in a plant plastid comprising expressing an exogenous nucleic acid in the plant that encodes an site-specific endonuclease targeted to the chloroplast to create a mutation in a chloroplast-encoded photosynthetic gene such that the plant is unable to perform photosynthesis. The non-photosynthetic plant is then grown as callus tissue and used as a recipient plant for complementation studies in which a gene conferring an agronomically and/or non-agronomically beneficial trait is introduced into the non-photosynthetic recipient plant plastids along with a functional copy of the mutated photosynthetic gene. A plant that is able to perform photosynthesis following complementation studies indicates that the plant plastid received the functional copy of the photosynthetic gene and by extension, the gene conferring the agronomically beneficial and/or non-agronomically trait. Embodiments of the disclosure provide for mutations in any gene present in the chloroplast genome. Other embodiments of the disclosure provide for plants produced by these methods, as well as plant parts. Elements, plants, reagents, or components used in the methods as described herein may also be provided in the form of a kit for introducing an agronomically and/or non-agronomically beneficial trait to a plant as described herein.

In some embodiments, the disclosure provides a method of expressing an agronomically beneficial trait in a plant plastid comprising: expressing an exogenous nucleic acid in the plant, wherein the exogenous nucleic acid is operably linked to a promoter functional in plants and a chloroplast transit peptide to target a site-specific endonuclease such as a Transcription Activator-Like Effector Nuclease (TALEN), Zinc Finger, TALE-cytosine deaminase (chloroTALE-DddA-UGI) or TALE-deoxyadenine deaminase, or restriction endonuclease to the chloroplast to create a mutation in a predefined chloroplast photosynthetic gene or genes, selecting a recipient mutant plant or plant part being homoplasmic for non-photosynthetic chloroplasts; growing a callus of the selected recipient mutant plant in culture; transforming the mutant plant-line callus with a plastid transformation vector comprising a wild-type copy of the mutated chloroplast gene and one or more agronomically and/or non-agronomically beneficial trait genes; and selecting green, photosynthetic callus; wherein the callus is grown in culture in light conditions. In some embodiments, one or more double-stranded breaks may be introduced into the chloroplast genome of the recipient cell such that a gene required for photosynthesis is disrupted (i.e., mutated) and the recipient plant can no longer perform photosynthesis. Suitable TALENs for use in the methods set forth herein can be designed by adapting disclosure of TALEN design set forth in Mahfouz et al. (2011) Proc. Natl. Acad. Sci. USA, 108:2623-2628; Mahfouz (2011) GM Crops, 2:99-103; as well as in U.S. Pat. Nos. 9,181,535, and 9,315,788, which are each incorporated herein by reference in their entireties. Suitable Zinc Finger endonucleases for use in the methods set forth herein can be designed by adapting disclosure of U.S. Pat. Nos. 6,453,242, 6,534,261, 6,479,626; 6,903,185; and 7,153,949, which are each incorporated herein by reference in their entireties. In other embodiments, TALE-cytosine deaminase (chloroTALE-DddA-UGI) or TALE-deoxyadenine deaminase can be used to create single nucleotide substitutions including mutations that create a stop codon in the coding region of the targeted gene or mutation that reduces or eliminates the function of the targeted gene to create the nonphotosynthetic phenotype.

Plastid genome encoded genes required for photosynthesis that can be targeted for disruption and/or complementation include, but are not limited to, the genes set forth below in Table 1 as well as functional equivalents thereof (e.g., genes encoding proteins have an enzymatic and/or biochemical activity of the gene set forth in Table 1) found in monocot (e.g., corn, wheat, rice, or sorghum) or dicot crop plants (e.g., cotton, soybean, tomato, or potato). Plastid genome encoded genes from maize and sorghum that can be targeted for disruption and/or complementation include genes set forth in Table 2. Plastid genome encoded genes from soybean that can be targeted for disruption and/or complementation include genes set forth in Table 3. Representative plastid genomes that have been sequenced and annotated with respect to the locations of plastid-encoded genes include: (1) Maize chloroplast genome (on the https world wide web site “ncbi.nlm.nih.gov/nucleotide/NC_001666.2;” (2) Sorghum chloroplast genome (on the https world wide web site “ncbi.nlm.nih.gov/nuccore/NC_008602.1;” (3) Soybean chloroplast genome (on the https world wide web site “ncbi.nlm.nih.gov/nuccore/NC_022868.1;” and (4) Rice chloroplast genome (on the https world wide web site “ncbi.nlm.nih.gov/nuccore/CP018170.1.”

TABLE 1
Plastid-encoded genes and conserved open reading frames (ycf = hypothetical
chloroplast reading frame) in seed plants and their essentiality under heterotrophic and
photoautotrophic conditions.

| | | Essential for | Essential for | |
|---|---|---|---|---|
| | | heterotrophic | autotrophic | |
| Gene | Gene product | growth | growth | Reference |
| psaA | A subunit of PSI | − | + | Redding et al., |
| | | | | 1999; C.r. |
| psaB | B subunit of PSI | − | + | Bock et al., 1999 |
| psaC | C subunit of PSI | − | + | Redding et al., |
| | | | | 1999; C.r. |
| ycf3 | Ycf3 protein, PSI assembly | − | + | Ruf et al., 1997 |
| ycf4 | Ycf4 protein, PSI assembly | − | −/+ | Krech et al., 2012 |
| psbA | D1 protein of PSII | − | + | Baena-González et |
| | | | | al., 2003 |
| psbB | CP47 subunit of PSII | − | + | |
| psbC | CP43 subunit of PSII | − | + | Rochaix et al., |
| | | | | 1989; C.r. |
| psbD | D2 protein of PSII | − | + | Erickson et al., |
| | | | | 1986; C.r. |
| psbE | α-subunit of cytochrome | − | + | Swiatek et al., 2003 a |
| | b₅₅₉ | | | |
| psbF | β-subunit of cytochrome | − | + | Swiatek et al., 2003 a |
| | b₅₅₉ | | | |
| psbH | H subunit of PSII | − | + | Erickson et al., |
| | | | | 1986; C.r. |
| psbJ | J subunit of PSII | − | + | Hager et al., 2002 |
| psbK | K subunit of PSII | − | + | Takahashi et al., |
| | | | | 1994; C.r. |
| psbL | L subunit of PSII | − | + | Swiatek et al., 2003 a |
| psbN/ | photosystem biogenesis | − | −/+ | Krech et al., 2013 |
| pbfl | | | | |
| petA | cytochrome f | − | + | Monde et al., 2000 |
| petB | cytochrome b₆ | − | + | Monde et al., 2000 |
| petD | subunit IV of cyt b₆f | − | + | Monde et al., 2000 |
| petG | G subunit of cyt b₆f | − | + | Schwenkert et al., |
| | | | | 2007 b |
| petN | N subunit of cyt b₆f | − | + | Hager et al., 1999 |
| atpA | ATP synthase α-subunit | − | + | Drapier et al., 1998; C.r. |
| atpB | ATP synthase β-subunit | − | + | Karcher and Bock, 2002 |
| atpE | ATP synthase ε-subunit | − | + | Karcher and Bock, 2002 |
| atpF | ATP synthase b-subunit | − | + | |
| atpH | ATP synthase c-subunit | − | + | unpublished |
| atpI | ATP synthase a-subunit | − | + | |
| rbcL | Rubisco large subunit | − | + | Kanevski and |
| | | | | Maliga, 1994 |
| accD | acetyl-CoA carboxylase | + | + | Kode et al., 2005 |
| | subunit | | | |
| ycf5/ccsA | subunit A of the system II | − | + | Xie and Merchant, |
| | complex for c-type | | | 1996; C.r. |
| | cytochrome biogenesis | | | |
| ycf1/ | 214 kDa protein of Tic | + | + | Drescher et al., |
| tic214 | complex | | | 2000; Kikuchi et al., |
| | | | | 2013 |
| ycf2 | putative Ycf2 protein | + | + | Drescher et al., 2000 |

PSI: photosystem I;
PSII: photosystem II;
cyt b₆f: cytochrome b₆f complex.

TABLE 2
Plastid-encoded genes from maize and sorghum. Sequences provided can be accessed on
the NCBI world wide web internet site “ncbi.nlm.nih.gov/genome/organelle/.” Plastid genes that are
adjacent to each other in the genome are separated by an empty row in the table.

| Adjacent | | Maize A188 chloroplast | Sorghum bicolor isolate |
|---|---|---|---|
| genes | Gene product | genome location | SbJ200 |
| psbK | K subunit of PSII | 7191..7376 | 7532..7717 |
| psbI | | | 8051..8206 |
| psbD | D2 protein of PSII | 9072..10133 | 9414..10475 |
| psbC | CP43 subunit of PSII | 10081..11502 | 10381..11844 |
| atpI | ATP synthase a-subunit | 32804..33547 | 33058..33801 |
| atpH | ATP synthase c-subunit | 34365..34610 | 34557..34802 |
| atpF | ATP synthase b-subunit | 35081..36463 | 35282..36672 |
| atpA | ATP synthase α-subunit | 36558..38081 | 36760..38283 |
| psaB | B subunit of PSI | complement(39103..41307) | complement(39333..41537) |
| psaA | A subunit of PSI | complement(41333..43585) | complement(41563..43815) |
| ycf3 | Ycf3 protein, PSI | complement(44193..46172) | complement(44412..46418) |
| | assembly | | |
| atpE | ATP synthase ┐- | complement(54182..54595) | complement(54408..54821) |
| | subunit | | |
| atpB | ATP synthase ┐- | complement(54592..56088) | complement(55376..56308) |
| | subunit | | |
| rbcL | Rubisco large subunit | 56870..58300 | 57110..58540 |
| ycf4 | Ycf4 protein, PSI | 59660..60217 | 59854..60411 |
| | assembly | | |
| petA | cytochrome f | 61479..62441 | 61705..62667 |
| psbJ | J subunit of PSII | complement(63341..63463) | complement(63567..63689) |
| psbL | L subunit of PSII | complement(63589..63705) | complement(63815..63931) |
| psbF | ┐-subunit of | complement(63728..63847) | complement(63954..64073) |
| | cytochrome b₅₅₉ | | |
| psbE | α-subunit of | complement(63858..64109) | complement(64084..64335) |
| | cytochrome b₅₅₉ | | |
| petG | G subunit of cyt b₆f | 65610..65723 | 65863..65976 |
| psbB | CP47 subunit of PSII | 70704..72230 | 70919..72445 |
| psbN/pbfl | photosystem biogenesis | complement(72553..72684) | complement(72788..72919) |
| psbH | H subunit of PSII | 72788..73009 | 73023..73244 |
| petB | cytochrome b₆ | 73139..74543 | 74081..74464 |
| petD | subunit IV of cyt b₆f | 74726..75952 | 75653..76177 |
| ycf5/ccsA | subunit A of the system | 109028..109993 | 109238..110203 |
| | II complex for c-type | | |
| | cytochrome biogenesis | | |
| psaC | C subunit of PSI | complement(111796..112041) | complement(112006..112251) |

TABLE 3
Plastid-encoded genes from soybean. Sequences provided can be accessed on the NCBI
world wide web internet site “ncbi.nlm.nih.gov/genome/organelle/.” Plastid genes that are adjacent
to each other in the genome are separated by an empty row in the table.

| Adjacent | | |
|---|---|---|
| Photosynthetic | | Glycine max chloroplast genome |
| Genes | Gene product | location (GenBank: NC_007942.1) |
| rbcL | Rubisco large subunit | complement(5312..6739) |
| atpB | ATP synthase ┐-subunit | 7522..9018 |
| atpE | ATP synthase ┐-subunit | 9015..9419 |
| ycf3 | Ycf3 protein, PSI assembly | 16430..18376 |
| psaA | A subunit of PSI | 18972..21224 |
| psaB | B subunit of PSI | 21250..23454 |
| psbC | CP43 subunit of PSII | complement(25671..27092) |
| psbD | D2 protein of PSII | complement(25671..27092) |
| petN | N subunit of cyt b₆f | complement(32118..32207) |
| atpI | ATP synthase a-subunit | 45971..46714 |
| atpH | ATP synthase c-subunit | 47818..48063 |
| atpF | ATP synthase b-subunit | 48505..49798 |
| atpA | ATP synthase α-subunit | 49868..51400 |
| psbK | K subunit of PSII | complement(54025..54210) |
| accD | acetyl-CoA carboxylase subunit | 56902..58200 |
| petA | cytochrome f | 60604..61566 |
| psbJ | J subunit of PSII | complement(62442..62564) |
| psbL | L subunit of PSII | complement(62705..62821) |
| psbE | α-subunit of cytochrome b₅₅₉ | complement(62973..63224) |
| psbF | ┐-subunit of cytochrome b₅₅₉ | complement(62844..62963) |
| petG | G subunit of cyt b₆f | 64388..64501 |
| psbB | CP47 subunit of PSII | 71019..72545 |
| psbN/pbfl | photosystem biogenesis | complement(72879..73010) |
| psbH | H subunit of PSII | 73124..73345 |
| petB | cytochrome b₆ | 73482..74936 |
| petD | subunit IV of cyt b₆f | 75133..76343 |
| psaC | C subunit of PSI | 120242..120487 |
| ycf5/ccsA | subunit A of the system II complex | complement(122382..123359) |
| | for c-type cytochrome biogenesis | |
| ycf1/tic214 | 214 kDa protein of Tic complex | complement(126634..127122) |
| ycf2 | putative Ycf2 protein | complement(142784..149647) |

In some embodiments, sequences targeted by the site-specific TALEN or Zinc-finger endonuclease can be chosen purposefully so that they are close to endogenous genomic sequences that carry homologous direct repeats or inverted repeat sequences. It is theorized that the double-strand break caused by the endonuclease can stimulate nearby homologous recombination between these repeated sequences, facilitating recovery of homoplasmic deletion mutants. Direct repeats or inverted repeats in the chloroplast genome can be identified by a variety of software applications, including Repeat Finder (on the internet site “tandem.bu.edu/trf/trf.html”) and einverted (on the internet site “emboss.open-bio.org/wiki/Appdocs”)

Plastid deletion mutants are expected to require standard 3% sugar in tissue culture for growth. Therefore, reduction of the sugar concentration or changing the sugar source, or selection on other media requiring photosynthesis for growth, can be performed to enable subsequent selection for photosynthetic competence.

An example of the deletion mutant and complementation strategy is shown in FIG. 2. In this case, the trait gene(s) of interest would be cloned adjacent to the complementing plastid fragment into a nearby intergenic region.

In certain embodiments, a deleted plastid region can be complemented with a perfect plastid gene copy and direct the integration of the transgene(s) to a different location in the plastid genome using a co-transformation approach. In this example, the co-transforming trait(s) would be carried on a second plasmid, and the trait gene(s) would be cloned into a suitable intergenic or non-coding region of the plastid genome. Integration of the trait gene(s) would be directed by homologous flanking regions that are homologous to the other region of the plastid genome.

RbcL encodes the large subunit of RuBisCo, the catalytic domain of the enzyme responsible for carboxylation during photosynthesis and arguably the most important enzyme in the world. Attempts to modify RuBisCo enzyme activity require chloroplast transformation technology. As shown in FIG. 2 and FIG. 5, TALEN cuts sites could be used to create a non-photosynthetic mutant by disruption of the chloroplast rbcL gene.

Complementation of rbcL mutant lines could be via a wild-type rbcL coding region to restore photosynthesis. Alternatively, complementation could be used to introduce an improved version of rbcL. For example, the maize rbcL could be replaced with the Synechocystis rbcL gene to generate autotrophic plants with higher CO2 fixation per unit enzyme compared to the maize rbcL, similar to Lin et al (2014) and Orr et al. (2019) that performed analogous experiments in tobacco chloroplasts. Additional improvements could include enhanced thermal stability of RuBisCo via A222T and V262L substitutions in rbcL, as shown in tobacco by Du and Spreitzer (2000). Several additional enhancement to photosynthesis via RuBisCo modifications could be envisioned as reviewed by Hanson et al. (2013).

Improvements in other chloroplast genes can be accomplished via the complementation strategy set forth herein. For example, resistance to several herbicides could be encoded in a modified psbA gene, encoding the D1 protein of Photosystem II. For example, resistance to triazine and other herbicides can be attained via change from Serine to glycine at position 264 of the protein (Tietjen et al. 1991). In some cases, modification of Valine 219 to isoleucine can result in resistance to DCMU, metribuzin and diuron herbicides (Mengistu et al. 2000). Other modifications to psbA may decrease photoinhibition (Larom et al. 2010) or increase tolerance to drought stress (Hu et al. 2016). More recently, it was shown that reductions in Chl b levels and light harvesting antenna size may have improved photosynthetic performance and biomass yield compared to wild-type plants (Friedland et al. 2019), which may be accomplished by modifications in psaA/B expression.

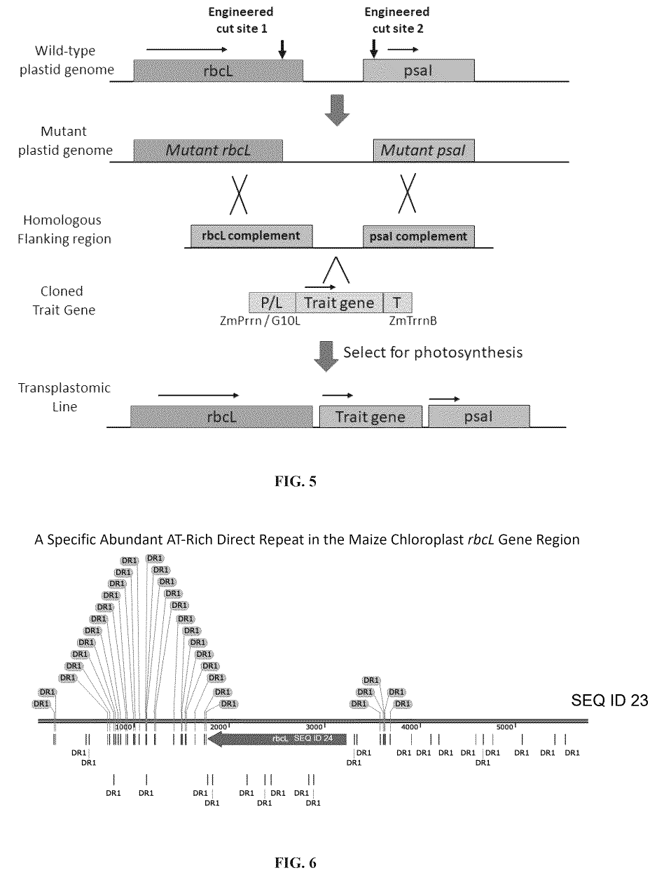

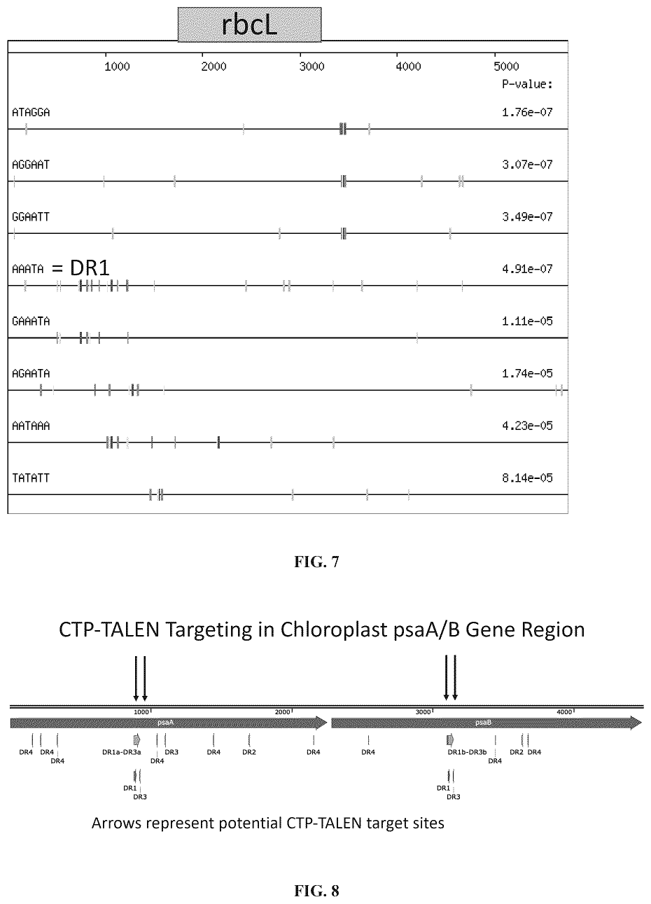

TALEN cut sites can be targeted to the rbcL gene sequence. In some cases, the TALEN cut sites can be targeted to specific sequences within the rbcL coding region that have sequence microhomologies to surrounding small repeated sequences in an effort to catalyze ectopic recombination with these sequences, resulting in larger deletions. In some other cases, the TALEN cut sites may not themselves have sequence microhomologies with small repeated sequences, though ectopic recombination among these repeated sequences may still catalyze larger deletions. The presence of direct repeat sequences in the rbcL region of the maize chloroplast genome was investigated using the RepFind software. I The RepFind software package (Betley et al., 2002; on the https internet site “zlab.bu.edu/repfind/form.html”) can be utilized for this analysis, though numerous software are available for searching of small repeated sequences in genomes including, for example, Tandem Repeat Finder on the https internet site “tandem.bu.edu/trf/trf.html”), equicktandem (on the http internet site “emboss.bioinformatics.nl/cgi-bin/emboss/equicktandem), einverted (on the http internet site “emboss.bioinformatics.nl/cgi-bin/emboss/einverted”) and palindrome (on the http internet site “emboss.bioinformatics.nl/cgi-bin/emboss/palindrome”). In certain embodiments, the analysis was limited to short (5-6 bp) AT-rich sequences, as these have previously been shown to catalyze ectopic recombination, as discussed above. The locations of a large number of a specific AT-rich repeated sequence surrounding the rbcL genomic region is shown in FIG. 6. FIG. 7 shows the location of additional repeat sequences in this region of the chloroplast genome. TALEN cut sites could be designed to incorporate AT sequences to potentially stimulate recombination with the direct repeat sequences, or these direct repeat sequences can catalyze ectopic recombination independent of TALEN cut site microhomology.

The chloroplast psaA and psaB photosynthetic genes are adjacent to each other in the maize chloroplast genome, and are co-transcribed and translationally coupled. Knockout of either gene creates a non-photosynthetic phenotype but is not lethal. TALEN-mediated deletion can be targeted to either of these two genes as described above and in FIG. 2. Alternatively, TALENs can be designed to target sites in both genes, such that deletion of both genes occurs, as illustrated in FIG. 5. In this case, complementation of both mutated genes can facilitate insertion of a trait gene between these two adjacent photosynthetic genes. Because the chloroplast genome is very compact, there are numerous examples of photosynthetic genes that are adjacent to each other as listed in Table 2 for monocots such as maize and sorghum and in dicots such as soybean in Table 3.

Another approach to creating a double-stranded break in the chloroplast genome is via digestion with a restriction endonuclease. In certain embodiments, a restriction endonuclease recognition site that is present only once in the chloroplast genome is used such that recombinational repair results in a simple deletion. In other cases, a restriction endonuclease that cuts twice or a small number of times in the chloroplast genome canbe used. Such restriction sites that occur once, twice, or more times in the plastid genome can be identified by analyzing the presence of restriction endonuclease recognition sites in sequenced plastid genomes. In certain embodiments, complementation of chloroplast non-photosynthetic plant cells results in a diagnostic new restriction endonuclease site or no site at all. A search of the maize chloroplast genome for a unique restriction enzyme sites was performed using the SnapGene software (on the internet https and world wide web site “snapgene.com”) though numerous software programs are available for a similar analysis. In the maize chloroplast genome, only 2 known restriction enzymes are found that have a single recognition site: AscI and FspA1. The recognition site for the AscI enzyme is fortuitously located in the chloroplast psaA/B gene coding region whereas the FspA1 enzyme recognition site is located in the psbB gene, so that deletions resulting from digestion of either of these enzymes should result in a non-photosynthetic mutant. The sorghum chloroplast genome contains a unique FspA1 site in the psbB photosynthetic gene. The FspA1 site and FspA1 restriction endonuclease can thus be used in the methods provided herein where sorghum plant cells are targeted. In the soybean chloroplast genome, a unique AbsI site resides in the psbD photosynthetic gene and a unique SfiI gene resides in the psbC photosynthetic gene. The AbsI and SfiI sites and AbsI and SfiI restriction endonucleases that thus be used in the methods provided herein where soyben plant cells are targeted.

In some embodiments, an exogenous nucleic acid as described herein may be operably linked to a promoter functional in plants and a chloroplast transit peptide. In this way, the exogenous nucleic acid encoding a site-specific endonuclease such as TALEN, Zinc Finger, TALE-cytosine deaminase (chloroTALE-DddA-UGI) or TALE-deoxyadenine deaminase or restriction endonuclease can be targeted to the chloroplast after expression. The exogenous site-specific endonuclease or nucleic acid encoding the same may be targeted to a particular gene in the chloroplast genome, such as a photosynthesis gene, which may result in inactivation or disruption of the chloroplast gene. A promoter useful in accordance with the disclosure may be any promoter functional in the nucleus or plastids of plants and appropriate for the particular application. In some embodiments, cells from the non-photosynthetic recipient plant may be cultured under sterile tissue culture conditions to produce callus tissue. The non-photosynthetic callus tissue may then be provided with one or more nucleic acid molecules such that the non-photosynthetic trait is complemented with the introduced nucleic acid. For example, providing the recipient plant with a functional copy of the mutated photosynthesis gene may be desired in some embodiments. In other embodiments, the functional copy of the photosynthesis gene may be provided to the recipient along with an agronomically beneficial and/or non-agronomically trait gene as desired.

In some embodiments, an exogenous nucleic acid encoding a site-specific endonuclease including a TALEN, Zinc Finger, or restriction endonuclease, or base editor TALE-cytosine deaminase (chloroTALE-DddA-UGI) or TALE-deoxyadenine deaminase, may be provided to the recipient cell in active form, or may be provided to the recipient cell as a nucleic acid encoding a desired gene, function, or trait. In some embodiments, such a nucleic acid encoding a site-specific endonuclease including a TALEN, Zinc Finger, or restriction endonuclease, or base editor TALE-cytosine deaminase (chloroTALE-DddA-UGI) or TALE-deoxyadenine deaminase may be provided to the cell in a transformation vector, such as a nuclear transformation or plastid transformation vector or other vehicle or delivery system (e.g., a viral vector expression system). In certain embodiments, the endonuclease can be provided in a transient expression system.

In some embodiments, a nucleic acid encoding a functional copy of a photosynthesis gene may be provided to the non-photosynthetic mutant plant cell or callus tissue comprising the same in order to complement the non-photosynthetic trait and result in a plant cell, plant callus, or sector in a plant, plant part, or callus that is able to perform photosynthesis and is therefore green in color. A gene conferring an agronomically and/or non-agronomically beneficial trait may be provided to the non-photosynthetic mutant plant callus along with the functional copy of the photosynthesis gene. As described above, nucleic acids may be provided to a plant or plant tissue in a vector or other vehicle or delivery system. In some embodiments, a transformation vector such as a plastid transformation vector may be used as appropriate. In some embodiments, a plastid transformation vector may comprise a chloroplast transformation vector. One of skill in the art will understand and be able to select a beneficial delivery system for use with the present disclosure.

As used herein, a “plastid transformation vector” or “chloroplast transformation vector” refers to a vector for transformation of a plastid as described herein. For example, a plastid transformation vector may be used as described herein for transformation of one or more chloroplasts of a plant having a non-photosynthetic phenotype. In such cases, the plastid transformation vector may be referred to as a chloroplast transformation vector. In accordance with the disclosure, a plastid transformation vector and a chloroplast transformation vector may be used interchangeably. As described herein, a chloroplast transformation vector may have a functional copy of a gene that was inactivated, mutated, or disrupted for expression in a non-photosynthetic mutant plant as described herein. A chloroplast transformation vector useful in accordance with the disclosure may also have at least a second gene for expression in a non-photosynthetic mutant plant as described herein, which may confer an agronomically and/or non-agronomically beneficial trait to the plant. In such a way, transformation of a non-photosynthetic plant may result in or produce a photosynthetic plant having an added agronomically and/or non-agronomically beneficial trait. Such traits are described herein elsewhere.

To ensure that the exogenous nucleic acid, functional copy of the photosynthesis gene, and/or gene conferring an agronomically beneficial trait and/or non-agronomically is expressed in the plant as desired, these elements may be operably linked to a promoter functional in plant. For example, a promoter useful with the disclosure may be a seed-specific promoter or an embryo-specific promoter. In another embodiment the promoter is functional in plant plastids. Any appropriate promoter may be used as long as the promoter is functional in plants or plant plastids.

In some embodiments, a plant useful for the disclosure may comprise any type of plant appropriate for the particular application, or to which a desirable or agronomically beneficial and/or non-agronomically trait is to be added. For example, a plant may be any crop or ornamental plant, such as including, but not limited to, soybean, corn, potato, wheat, or the like. In some embodiments, the plant may be a monocot or a dicot species. Some embodiments provide particular benefit to transformation of monocot species, which have thus far lacked effective methods for plastid transformation. Monocot species that may be particularly useful may include corn, wheat, rice, sorghum, Asparagus, sugarcane, onion, garlic, or the like. In some embodiments, the disclosure may be useful for transformation of plastids, e.g., chloroplasts, in a monocot plant or a dicot plant. In some specific embodiments, the plant may be, for example, a corn plant, a sorghum plant, or a soy plant. In some embodiments, introducing an exogenous nucleic acid encoding a site-specific endonuclease into a plant or plant cell will produce a plant or plant cell in which a gene has been interrupted or mutated. Such a plant or plant cell may be referred to herein as a “recipient mutant plant or plant cell line,” “recipient plant or plant cell line,” “recipient,” or a “recipient homoplasmic non-photosynthetic plant or plant cell,” and the like. Such a plant may, in accordance with certain embodiments, have a mutation in a photosynthetic gene such that the recipient plant cannot perform photosynthesis under light conditions at wild-type levels and will have a non-green phenotype. By “non-green” is meant a plant that is deficient, completely or in part, in photosynthesis, and therefore cannot produce chlorophyll, leading to an absence of green pigmentation in the plant. Such plants may also be referred to herein as “non-photosynthetic.” Non-photosynthetic “loss-of-function” mutations thus include mutations which confer a complete or partial loss-of-function of the gene (e.g., 0%, 5%, 10%, 20%, 30%, or 40% or less of wild-type activity; or alternatively, 0%, 5%, or 10% to 15%, 20%, or 40% of wild-type activity). For example, as described herein, a mutation in a chloroplast gene confers a non-green (i.e., non-photosynthetic) phenotype when grown under light conditions. A non-photosynthetic plant as described herein may be any level of non-green, i.e., white, pale green, yellow-green, or the like. “Non-green” refers to a phenotypic color of the plant tissue that has less green pigment (i.e., is less green) that a wild-type plant that is able to perform photosynthesis.

In some embodiments, non-photosynthetic mutants as described herein may be maintained on a rich media source. Any rich media source appropriate for growth of plant tissue or cells may be used. In some embodiments, non-photosynthetic mutants as described herein can be maintained on a media which comprises a sugar, an organic acid, or a combination thereof that supports growth of the whole plant, whole plant seedling, or whole plant part. In some embodiments, a non-photosynthetic mutant may not grow on media that requires active photosynthesis for survival. In other embodiments, a non-photosynthetic mutant may grow poorly on media that requires active photosynthesis for survival.

In some embodiments, a plant as described herein may be grown under dark conditions, referring to conditions under which photosynthesis will not normally occur. In other embodiments, a plant as described herein may be grown under light conditions. Light conditions as used herein is in reference to conditions under which photosynthesis would normally occur. As would be understood by one of skill in the art, growth conditions such as lighting may be altered to suit a particular plant species or to take advantage of the needs of a particular species. For example, as described herein, some plants having a mutation in a gene required for photosynthesis, either plastid-encoded or nuclear-encoded, will have altered function as compared to a wild type plant. In a particular, non-limiting example, a plant having a mutation in a photosynthesis gene, referred to herein as a non-photosynthetic mutant, may be unable to perform photosynthesis under typical high-light conditions usually used for growth of wild-type plants. In accordance with the disclosure, growth conditions may be altered as necessary for the particular non-photosynthetic mutant. For example, a particular non-photosynthetic mutant may still be able to accumulate chlorophyll and thus have a reduced level of green color under dim light conditions. Such mutant plant lines may grow slowly under dim light, but would grow more poorly and begin to bleach and lose green color if transferred from dim light to a bright light on media requiring photosynthesis. Such a method may allow non-photosynthetic mutant plant lines to grow and amplify enough to perform plastid transformation experiments. In contrast, wild-type cells would be expected to grow fast and turn green under both high-light and dim-light conditions. Alternatively, non-photosynthetic mutant lines may be grown in the dark as typical callus to amplify material for plastid transformation experiments. Such mutant plant lines may then be shifted into the light to select for plastid transformed plants where photosynthesis is required for growth. Non-photosynthetic mutant callus would remain non-green in high-light or only slowly turn green under dim light, whereas plastid transformed cells would turn fully green under these same conditions. In contrast, wild-type callus would turn green when shifted from the dark to any light condition.

In some embodiments, a non-photosynthetic plant as described herein may be a homoplasmic chloroplast mutant plant line. As used herein, “homoplasmic” refers to a plant in which all copies of the chloroplast or plastid genome are identical. Homoplasmy may occur naturally in a plant, or it may be artificially induced using methods known in the art. For example, as described herein, a gene present in the chloroplast genome may be modified, interrupted, mutated, altered, eliminated, etc., using genetic engineering methodology, which is well-known in the art. In some embodiments, a non-photosynthetic homoplasmic chloroplast mutant plant line may comprise a mutation in a chloroplast gene as described herein. Such a mutation may be introduced by any methods known in the art.

As described herein, a chloroplast gene may be modified or altered such that the gene is non-functional, resulting in a non-photosynthetic plant. Such a plant or plant part may then be used as a recipient for complementation studies wherein a functional copy of a mutated chloroplast gene is introduced into the plant along with a gene conferring a trait of interest, such as an agronomically beneficial and/or non-agronomically trait. In this way, introduction of the functional chloroplast gene and the gene of interest into the recipient plant may restore photosynthesis in the recipient plant, as well as provide the agronomically beneficial and/or non-agronomically trait to the plant. Thus, in some embodiments, complementation as described herein may be used as a marker of integration of a trait of interest into a recipient plant. Such a marker may eliminate the need for a further selection step, such as antibiotic selection to identify effectively transformed plants, referred to herein as “transformants.”

In another example, when mutant non-photosynthetic cultures are used, either nuclear-encoded mutants or chloroplast deletion mutants, the cultures are already non-green or pale-green on media that requires photosynthesis for greening. In this case, the mutant may be complemented using expression of the nuclear gene in chloroplasts or the restoring chloroplast gene in chloroplasts and therefore the selection step is for photosynthesis, growth, and green on media that requires photosynthesis. The “selectable marker,” then, is the nuclear gene or the restoring chloroplast gene.

A plant having a mutation in a chloroplast protein may be referred to herein as a “mutant plant” or a “mutant line.” Mutant lines as described herein may be produced using the methods of the disclosure and may be used as recipient plant lines for complementation studies as described herein.

Plant breeding programs are well-known in the art and can be modified as necessary depending on the growth requirements of the particular plant species used. For example, in some embodiments, a mutation producing a non-photosynthetic mutant plant in accordance with the disclosure may be maintained in a hybrid or an inbred genetic background, including elite inbred genetic backgrounds. Any genetic background may be used in accordance with the disclosure, as long as the desired mutation is able to be maintained for the desired use.

As used herein, a “promoter” refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ-or cell-specific promoters are expressed only or predominantly in a particular tissue, organ, or cell type, respectively. Rather than being expressed “specifically” in a given tissue, plant part, or cell type, a promoter may display “enhanced” expression, i.e., a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

As described herein, a plant useful for any of the methods of the disclosure may comprise any type of plant appropriate for the particular application, or to which a desirable or agronomically beneficial and/or non-agronomically trait is to be added. Any crop or ornamental plant may be used, such as including, but not limited to, soybean, corn, potato, wheat, sorghum, rice, or the like. In some specific embodiments, the plant may be, for example, a corn plant or a soy plant.

In some embodiments, a plant in which a gene has been interrupted may be referred to herein as a “recipient mutant plant line” or a “recipient plant line” or a “recipient.” Such a plant may, in certain embodiments, have a mutation in a nuclear-encoded photosynthetic gene such that the recipient plant cannot perform photosynthesis under light conditions and will have a non-green phenotype. By “non-green” is meant a plant that is deficient, completely or in part, in photosynthesis, and therefore cannot produce chlorophyll, leading to an absence or reduction of green pigmentation in the plant. Such plants may also be referred to herein as “non-photosynthetic.” A non-photosynthetic plant as described herein may be any level of non-green, i.e., white, pale green, yellow-green, or the like . . . “Non-photosynthetic “loss-of-function” mutations in nuclear-encoded photosynthetic genes thus include mutations which confer a complete or partial loss-of-function of the gene (e.g., 0%, 5%, 10%, 20%, 30%, or 40% or less of wild-type activity; or alternatively, 0% or 5%, or 10%, 15%, 20%, or 40% of wild-type activity). “Non-green” refers to a phenotypic color of the plant tissue that has less green pigment (i.e., is less green) that a wild-type plant that is able to perform photosynthesis.

In some embodiments, a non-photosynthetic plant as described herein may be a homozygous non-photosynthetic mutant, having both non-functional copies of the nuclear-encoded photosynthesis gene. In other embodiments, it may be beneficial to perform certain steps of the methods described herein in a heterozygous plant. In some embodiments, a non-photosynthetic mutant plant may also be a homoplasmic chloroplast mutant plant line. As used herein, “homoplasmic” refers to a plant in which all copies of the chloroplast or plastid genome are identical. Homoplasmy may occur naturally in a plant, or it may be artificially induced using methods known in the art.

As described herein, a gene required for photosynthesis may be modified, interrupted, mutated, altered, eliminated, etc., using genetic engineering methodology. A plant in which such a mutation is introduced may then be used as a recipient for complementation studies wherein a functional copy of a mutated photosynthesis gene is introduced into the plant along with a gene conferring a trait of interest, such as an agronomically beneficial and/or non-agronomically trait. In this way, introduction of the functional gene and the gene of interest into the recipient plant chloroplast may restore photosynthesis in the recipient plant, as well as provide the desired agronomically beneficial and/or non-agronomically trait to the plant. Thus, in some embodiments, complementation as described herein may be used as a marker of integration of a trait of interest into a recipient plant. Such a marker may eliminate the need for a further selection step, such as antibiotic selection to identify effectively transformed plants, referred to herein as “transplastomic” plants or plant cells or “transformants.”

In some embodiments, a mutant line as described herein may be maintained in a particular genetic background, in order to maintain plants with the desired genetic mutation. For example, as described herein, a mutant line may be maintained in a hybrid genetic background. Plants heterozygous for a genetic mutation as described herein may carry the non-photosynthetic mutation while exhibiting a photosynthetic phenotype. Plant breeding programs may be used to maintain a desired genetic mutation in a particular plant line or genetic background. Plant breeding programs are well-known in the art and can be modified as necessary depending on the growth requirements of the particular plant species used. For example, in some embodiments and as appropriate for the particular mutation, a mutation producing a non-photosynthetic mutant plant in accordance with the disclosure may be maintained in a hybrid genetic background such as including, but not limited to, an A188 x B73 hybrid genetic background. Any genetic background may be used in accordance with the disclosure, as long as the desired mutation is able to be maintained for the desired use.

In some embodiments, a method as described herein may further comprise directly transforming a non-photosynthetic plastid as described herein, for example using a chloroplast transformation vector as described herein. Such a vector may have a functional copy of a gene that was disrupted or mutated such that the functional copy may replace the non-functional gene. In some embodiments, the function of the inactivated gene may be complemented by the presence of the functional copy, for example upon expression of the functional copy of the gene from the chloroplast transformation vector. In other embodiments, the functional copy of the gene may be incorporated into the chloroplast genome and restore function of the gene in that way. In some embodiments, the chloroplast transformation vector may also have a second gene that confers to the plant an agronomically and/or non-agronomically beneficial trait or phenotype as described herein.

In some embodiments, the disclosure also provides a non-photosynthetic chloroplast produced by a method as described herein.

Polynucleotides useful in the present disclosure can be provided in an expression construct. Expression constructs of the disclosure generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements used for expression of nuclear genes include promoters, transcription termination sequences, translation termination sequences, enhancers, and polyadenylation elements. Regulatory elements used for expression of plastid genes include promoters, translational leader sequences, transcription stability and termination sequences and translation termination sequences. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the disclosure can comprise a promoter sequence operably linked to a polynucleotide sequence encoding a polypeptide of the disclosure. Promoters can be incorporated into a polynucleotide using standard techniques known in the art. Multiple copies of promoters or multiple promoters can be used in an expression construct of the disclosure. In one embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

If the expression construct is to be provided in or introduced into a plant cell nucleus, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, prolifera promoter, Ap3 promoter, heat shock promoters, T-DNA 1′-or 2′-promoter of A. tumefaciens, polygalacturonase promoter, chalcone synthase A (CHS-A) promoter from petunia, tobacco PR-la promoter, ubiquitin promoter, actin promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used. Tissue-specific promoters, for example xylem-specific promoters, such as the promoter of Cald5H, SAD, XCPI, CAD, CesA1, CesA2, CesA3, tubulin gene (TUB) promoter, lipid transfer protein gene (LTP) promoter, or coumarate-4-hydroxylase gene (C4H) promoter (see, for example, Lu et al., 2008; Funk et al., 2002; Sibout et al., 2005; published U.S. application no. 2008/0196125) can be used. Leaf-specific promoters that can be used in a nucleic acid construct of the disclosure include Cab1 promoter (Brusslan and Tobin, 1992), Cab19 promoter (Bassett et al., 2007), PPDK promoter (Matsuoka et al., 1993), and ribulose biphosphate carboxylase (RBCS) promoter (Matsuoka et al. (1994) and U.S. Pat. No. 7,723,575). Other plant leaf-specific promoters that can be used with an expression construct of the disclosure include, but are not limited to, the Act1 promoter (U.S. Published application No. 20090031441), AS-1 promoter (U.S. Pat. No. 5,256,558), RBC-3A promoter (U.S. Pat. No. 5,023,179), the CaMV 35S promoter (Odell et al., 1985), the enhanced CaMV 35S promoter, the Figwort Mosaic Virus (FMV) promoter (Richins et al., 1987), the mannopine synthase (mas) promoter, the octopine synthase (ocs) promoter, or others such as the promoters from CaMV 19S (Lawton et al., 1987), nos (Ebert et al., 1987), Adh (Walker et al., 1987), sucrose synthase (Yang et al., 1990), α-tubulin, ubiquitin, actin (Wang et al., 1992), cab (Sullivan et al., 1989), PEPCase (Hudspeth et al., 1989) or those associated with the R gene complex (Chandler et al., 1989). See also published U.S. application 2007/006346 and Yamamoto et al. (1997); Kwon et al. (1994); Yamamoto et al. Other promoters that direct expression in the xylem of plants include the 4-coumarate Co-enzyme A ligase (4CL) promoter of Populus described in U.S. Pat. No. 6,831,208. Seed-specific promoters such as the promoter from a β-phaseolin gene (for example, of kidney bean) or a glycinin gene (for example, of soybean), and others, can also be used. Endosperm-specific promoters include, but are not limited to, MEG1 (EPO application No. EP1528104) and those described by Wu et al. (1998), Furtado et al. (2001), and Hwang et al. (2002). Root-specific promoters, such as any of the promoter sequences described in U.S. Pat. No. 6,455,760 or U.S. Pat. No. 6,696,623, or in published U.S. patents application Nos. 20040078841; 20040067506; 20040019934; 20030177536; 20030084486; or 20040123349, can be used with an expression construct of the disclosure. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs of the disclosure.

Methods for identifying and characterizing promoter regions in plant genomic DNA are known in the art and include, for example, those described in the following references: Jordano et al. (1989); Bustos et al. (1989); Green et al. (1988); Meier et al. (1991); and Zhang et al. (1996). U.S. Application Publication No. 2009/0199307 also describes methods for identifying tissue-specific promoters using differential display (see, e.g., U.S. Pat. No. 5,599,672). In differential display, mRNAs are compared from different tissue types. By identifying mRNA species which are present in only a particular tissue type, or set of tissue types, corresponding genes can be identified which are expressed in a tissue specific manner. RNA can be transcribed by reverse transcriptase to produce a cDNA, and the cDNA can be used to isolate clones containing the full-length genes. The cDNA can also be used to isolate homeologous or homologous promoters, enhancers or terminators from the respective gene using, for example, suppression PCR. See also U.S. Pat. No. 5,723,763.

Nuclear Expression constructs of the disclosure may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the disclosure. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences that direct polyadenylation of mRNA transcribed from the expression construct can also be included in the expression construct, and include, but are not limited to, an octopine synthase or nopaline synthase signal.

Polynucleotides of the present disclosure can be composed of either RNA or DNA. In certain embodiments, the polynucleotides are composed of DNA. The subject disclosure also encompasses those polynucleotides that are complementary in sequence to the polynucleotides disclosed herein. Polynucleotides and polypeptides of the disclosure can be provided in purified or isolated form.

Because of the degeneracy of the genetic code, a variety of different polynucleotide sequences can encode polypeptides of the present disclosure. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides of the subject disclosure. These variant or alternative polynucleotide sequences are within the scope of the subject disclosure. As used herein, references to “essentially the same” sequence refers to sequences which encode amino acid substitutions, deletions, additions, or insertions which do not materially alter the functional activity of the polypeptide encoded by the polynucleotides of the present disclosure.

Amino acids can be generally categorized in the following classes: non-polar, uncharged polar, basic, and acidic. Conservative substitutions whereby a polypeptide of the present disclosure having an amino acid of one class is replaced with another amino acid of the same class fall within the scope of the subject disclosure so long as the polypeptide having the substitution still retains substantially the same functional activity as the polypeptide that does not have the substitution. Polynucleotides encoding a polypeptide having one or more amino acid substitutions in the sequence are contemplated within the scope of the present disclosure.

Expression of plastid genes is different from nuclear genes. Plastid gene expression signals include a promoter, translational control region, coding sequence and transcription stability sequence. Plastid transgene expression signals are derived from resident plastid genes, or in some cases, can be derived from bacterial genes or from bacteriophage genes.

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules are capable of forming an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Stringency conditions are known in the art and would be understood by one of skill reading the present disclosure. One of skill in the art will also understand that stringency may be altered as appropriate to ensure optimum results. Complementarity as described herein also refers to the binding of a DNA editing enzyme to its target in vivo or in vitro. One of skill in the art would recognize that variations in complementarity will depend on the particular nucleic acid sequence and will be able to modify conditions as appropriate to account for this.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

Polynucleotides and polypeptides contemplated within the scope of the subject disclosure can also be defined in terms of more particular identity and/or similarity ranges with those sequences of the disclosure specifically exemplified herein. The sequence identity will typically be greater than 60%, greater than 75%, greater than 80%, greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength =12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single-or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

In some embodiments, the disclosure provides plants generated by a method as described herein. For example, in some embodiments, the disclosure provides plants produced by inactivating a chloroplast-encoded gene as described herein. In other embodiments, the disclosure is also intended to encompass plants produced by inactivating a nuclear-encoded gene that controls a chloroplast-encoded gene as described herein. In such cases, the nuclear-encoded gene may encode a gene involved directly as a core component of the chloroplast photosynthetic machinery or a transcription factor or a translation factor that serves to control a gene encoded by the chloroplast and is required for photosynthesis. Plants may be produced using any methods known in the art, and using any appropriate growth conditions and/or reagents. One of skill in the art will understand and be able to perform plant growth techniques and methods. In some embodiments, callus may be generated from a non-photosynthetic mutant plant as described herein using standard protocols known in the art. As described herein, a non-photosynthetic mutant plant of the disclosure is intended to encompass a plant in which a chloroplast-encoded gene is inactivated or disrupted, as well as a plant in which a nuclear-encoded gene is inactivated or disrupted that controls a chloroplast gene required for photosynthesis.

As described herein, callus tissue may be generated from a non-photosynthetic mutant plant or plant parts. As would be understood by one of skill in the art, callus may be generated from any type of plant tissue, for example, embryo tissue, seeds, such as seed explant tissue, stem, meristem, leaf, root, etc. Callus generated from such plants and grown in culture may then be used as recipients for complementation experiments as described herein. Other embodiments provide a plant part of a plant as described herein, selected from the group consisting of embryo, seed, stem, callus, meristem, leaf, root, or any plant part from which viable cells may be obtained and used in culture. In another embodiment, the disclosure provides a seed produced by a plant as described herein. Some embodiments provide for a plant regenerated from a callus, or any part of a plant resulting from a mutant, non-photosynthetic plant as described herein. Other embodiments provide a plant part of a plant as described herein, selected from the group consisting of a seed, stem, callus, meristem, leaf, root, or the like. In another embodiment, the disclosure provides a seed produced by a plant as described herein.

Nuclear transformation in monocots typically requires embryogenic callus or scutellar tissue from freshly isolated immature embryos, as these are the most highly regenerable tissues in monocot cell culture. Recently, it has been shown that over-expression of maize Baby boom (Bbm) and Wuschel (Wus) genes can rapidly initiate embryogenic callus growth from a variety of monocot tissues, leading to stable nuclear transformation from tissues such as mature seed or leaf segments. Furthermore, Bbm and Wus gene over-expression can enable nuclear transformation in otherwise recalcitrant genotypes (Lowe et al. 2016). Chloroplast non-photosynthetic mutants are anticipated to grow as embryogenic callus on media containing sugars. It is anticipated that over-expression of Bbm and Wus genes can also enhance the growth of these non-photosynthetic mutants or enhance selection for greening and photosynthetic competence in embryogenic callus and other tissues such as leaf. In certain embodiments, Bbm and Wus polypeptides and genes set forth in US Patent Appl. Pub. No. 20170342431; and U.S. Pat. No. 7,256,322, both incorporated herein by reference in their entirety, can be adapted for use in the methods provided herein to provide for plastid transformation and regeneration of transplastomic plants.

In some embodiments, the plants of the present disclosure may also further exhibit one or more agronomically and/or non-agronomically beneficial traits. As used herein, an “agronomically beneficial trait” refers to a trait or characteristic that is desirable or important for a particular crop or species. Agronomically beneficial traits may result in increased commercial value of the plant or crop, such as by providing improved taste, resistance to herbicides or pests, environmental tolerance, or may provide a selective advantage for the plant. Such traits may be desirable for, for example, a seed company, a grower, or a grain processor. Agronomically beneficial traits include traits effected by genes that alter quality traits such as oil (e.g.; fatty acid), protein, amino acid, starch, and/or other nutrient content and/or profiles in plant products including seed and seed meal. Agronomically beneficial traits include traits effected by genes, that provide for improved processing or use of plant products including seed meal, defatted seed meal, and the like as food or animal feed products. Examples of agronomically beneficial traits may include any desired characteristic, such as including, but not limited to, herbicide resistance (e.g., tolerance to glyphosate, glufosinate, dicamba or other auxin analogs), virus resistance, bacterial pathogen resistance, insect resistance, nematode resistance, and fungal resistance (e.g., abiotic-stress tolerance traits). Such a trait may also be one that increases plant vigor or yield, including traits that allow a plant to grow at different temperatures, soil conditions, and levels of sunlight and precipitation (e.g., abiotic-stress tolerance traits), or one that allows identification of a plant exhibiting a trait of interest (e.g., selectable marker gene, flower color or pattern, seed coat color, etc.). Various traits of interest, as well as methods for introducing these traits into a plant, are known in the art. For example, herbicide resistant results from over-expression of EPSPS, HPPD, bar, or AHAS herbicide resistant genes, cry1A, cry2A, or cry3A crystal proteins derived from Bacillus thuringiensis to provide insect resistance, defensins, or anti-fungal peptides to provide resistance to bacterial and fungal species. Non-agronomically beneficial traits include genes that encode proteins or enzymes that can be used in pharmaceutical or industrial applications.

Methods of plant transformation are well-known in the art. Techniques for transforming plant cells with a gene include, for example, Agrobacterium infection, biolistic methods, electroporation, DNA coated particles, calcium chloride treatment, PEG-mediated transformation, etc. (see, e.g., Nagel et al., 1990; Song et al., 2006; de la Pena et al., 1987; and Klein et al., 1993). U.S. Pat. No. 5,661,017 teaches methods and materials for transforming an algal cell with a heterologous polynucleotide. Suitable methods for transformation of host cells for use with the current disclosure are believed to include virtually any method by which DNA can be introduced into a cell (see, e.g., Miki et al., 1993), for example by Agrobacterium-mediated transformation (U.S. Pat. Nos. 5,563,055; 5,591,616; 5,693,512; 5,824,877; 5,981,840; 6,384,301; Gelvin, 2003; and Broothaerts et al., 2005) and by acceleration of DNA coated particles (U.S. Pat. Nos. 5,015,580; 5,550,318; 5,538,880; 6,160,208; 6,399,861; and 6,403,865), etc. Through the application of techniques such as these, the cells of virtually any species may be stably transformed.

In some embodiments, plant transformation can be achieved by biolistic transformation, or bombardment. As used herein, bombardment refers to a method of insertion of genetic material into a cell wherein the genetic material is forcibly injected into the cell with the use of a biolistic gun or related device or vehicle. Using biolistic transformation, a plasmid carrying a wild-type chloroplast genome segment as described herein may be delivered to a cell, such that integration of the transforming DNA complements the deletion mutant and restores the ability of the cell to perform photosynthesis. Photosynthetic capability may be verified by the ability of the cell or callus to grow on selective media. Selective media are widely known and available in the art. Media for cell culture may be altered as appropriate for the particular application without altering the scope of the disclosure as described herein.

Following transformation, cells can be selected, re-differentiated or regenerated, and grown as callus or grown into plants that contain and express a polynucleotide of the disclosure using standard methods known in the art. The seeds and other plant tissue and progeny of any transformed or transgenic plant cells or plants of the disclosure are also included within the scope of the present disclosure.

Various methods for selecting transformed cells have been described. For example, one might utilize a drug resistance marker such as a neomycin phosphotransferase protein to confer resistance to kanamycin or to use 5-enolpyruvyl shikimate phosphate synthase to confer tolerance to glyphosate. In another embodiment, a carotenoid synthase is used to create an orange pigment that can be visually identified. These three exemplary approaches can each be used effectively to isolate a cell or multicellular organism or tissue thereof that has been transformed and/or modified as described herein. In some embodiments, the present methods may eliminate the need for a selection step with the generation of mutant non-photosynthetic plants as described herein.

Numerous permutations of methods for biolistics for plant transformation are known and available in the art. For example, in some embodiments, bombardment and selection experiments of dark-grown callus may be performed as described herein by bombardment of dark-grown callus, followed by selection in the light, on media requiring photosynthesis for growth, until green callus (putative transformed lines) is observed. In other embodiments, bombardment of dark-grown callus shifted into the light for several days until such time that greening of wild-type callus would normally occur, followed by selection in the light, on media requiring photosynthesis for growth, until green callus (putative transformed lines) is observed. In some embodiments, bombardment of dark-grown callus may be followed by immediate plant regeneration on media requiring photosynthesis for growth, and any resulting green regenerated plants may be evaluated as having been complemented.

The direct selection of green photosynthetically competent calli described above is in contrast to the statements of Hajj et al. (2018) that indicates, “In higher plants, direct selection for restoration of photosynthesis is not possible. This is because early in the transformation process cells and tissues are propagated on media containing sucrose. This allows both transformed cells and nontransformed mutant cells to proliferate during the early stages of transformation. Following regeneration, nontransformed shoots vastly outnumber transplastomic shoots hindering their identification. Selection for photosynthesis is possible once shoots are moved to media lacking sucrose, which is difficult to achieve with large numbers of shoots in vitro.” It should be noted media containing sucrose was used in the instant inventor's experiments and green calli were readily observed, indicating that direct selection for restoration of photosynthesis is indeed possible.

In some embodiments, a non-naturally occurring sequence-specific or sequence-directed exogenous nucleic acid is introduced into a cell in order to introduce a mutation in a gene required for photosynthesis in the cell, or in an organism comprised of such cells. In some embodiments, the cell is a plant cell and the mutation results in the inability of the cell to undergo photosynthesis. The ability to generate such a cell, or an organism derived therefrom depends on introducing an exogenous nucleic acid into the cell using, for example, transformation vectors and cassettes described herein.

A polypeptide useful in accordance with the disclosure may be isolated, non-naturally occurring, recombinant, or engineered nucleic acid-binding proteins that have nucleic acid or DNA binding regions containing polypeptide monomer repeats designed to target specific nucleic acid sequences.

### Transient Expression of Exogenous Nucleic Acids

In some embodiments, an exogenous nucleic acid as described herein may be transiently introduced into the cell. In certain embodiments, the introduced exogenous nucleic acid is provided in sufficient quantity to modify the cell but does not persist after a contemplated period of time has passed or after one or more cell divisions. In such embodiments, no further steps are needed to remove or segregate the exogenous nucleic acid from the modified cell.

In another embodiment, mRNA encoding the exogenous nucleic acid is introduced into a cell. In such embodiments, the mRNA is translated to produce the exogenous nucleic acid in sufficient quantity to modify the cell but does not persist after a contemplated period of time has passed or after one or more cell divisions. In such embodiments, no further steps are needed to remove or segregate the exogenous nucleic acid from the modified cell.

In one embodiment of this disclosure, a catalytically active exogenous nucleic acid is prepared in vitro prior to introduction to a cell, including a prokaryotic or eukaryotic cell. The method of preparing a exogenous nucleic acid depends on its type and properties and would be known by one of skill in the art. After expression, the exogenous nucleic acid is isolated, refolded if needed, purified and optionally treated to remove any purification tags, such as a His-tag. Once crude, partially purified, or more completely purified exogenous nucleic acid are obtained, it may be introduced to, for example, a plant cell via electroporation, by bombardment with coated particles, by chemical transfection or by some other means of transport across a cell membrane as described herein. Methods for introducing nucleic acids into bacterial and animal cells are similarly well known in the art. In the case of Agrobacterium-mediated plant transformation methods, the exogenous nucleic acid can be expressed in Agrobacterium as a recombinant protein, fused to an appropriate domain of a Vir protein such that it is transported to the plant cell (Vergunst et al., 2000). The protein can also be delivered using nanoparticles, which can deliver a combination of active protein and nucleic acid (Torney et al., 2007). Once a sufficient quantity of the exogenous nucleic acid is introduced so that an effective amount is present, the target site or sites are looped out. It is also recognized that one skilled in the art might create an exogenous nucleic acid that is inactive but is activated in vivo by native processing machinery.

In another embodiment, a construct that will transiently express a exogenous nucleic acid is created and introduced into a cell. In yet another embodiment, the vector will produce sufficient quantities of the exogenous nucleic acid in order for the desired target site or sites to be effectively recombined. For instance, the disclosure contemplates preparation of a vector that can be bombarded, electroporated, chemically transfected or transported by some other means across the plant cell membrane. Such a vector could have several useful properties. For instance, in one embodiment, the vector can replicate in a bacterial host such that the vector can be produced and purified in sufficient quantities for a transient expression. In another embodiment, the vector can encode a drug resistance gene to allow selection for the vector in a host, or the vector can also comprise an expression cassette to provide for the expression of the exogenous nucleic acid in an organism. In a further embodiment, the expression cassette could contain a promoter region, a 5′ untranslated region, an optional intron to aid expression, a multiple cloning site to allow facile introduction of a sequence encoding an exogenous nucleic acid, and a 3′ UTR. In some embodiments, it can be beneficial to include unique restriction sites at one or at each end of the expression cassette to allow the production and isolation of a linear expression cassette, which can then be free of other vector elements. The untranslated leader regions, in certain embodiments, can be plant-derived untranslated regions. Use of an intron, which can be plant-derived, is contemplated when the expression cassette is being transformed or transfected into a monocot cell.

As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. As used herein, an expression cassette may comprise an exogenous nucleic acid operably linked to a promoter as described herein and a chloroplast transit peptide.

In another approach, a transient expression vector may be introduced into a cell using a bacterial or viral vector host. For example, Agrobacterium is one such bacterial vector that can be used to introduce a transient expression vector into a host cell. When using a bacterial, viral or other vector host system, the transient expression vector is contained within the host vector system. For example, if the Agrobacterium host system is used, the transient expression cassette would be flanked by one or more T-DNA borders and cloned into a binary vector. Many such vector systems have been identified in the art (reviewed in Hellens et al., 2000). In embodiments whereby the exogenous nucleic acid is transiently introduced in sufficient quantities to modify a cell, a method of selecting the modified cell may be employed. In the present case, one may look for non-green plant sectors or non-green embryo tissues or callus derived from embryos. In one such method, a second nucleic acid molecule containing a selectable marker may be co-introduced with the transient exogenous nucleic acid.

### Stable Expression of Exogenous Nucleic Acids

Cell transformation systems have been described in the art and descriptions include a variety of transformation vectors. For example, for plant transformations, two principal methods include Agrobacterium-mediated transformation and particle gun bombardment-mediated transformation. In both cases, the exogenous nucleic acid is introduced via an expression cassette. The cassette may contain one or more of the following elements: a promoter element that can be used to express the exogenous nucleic acid; a 5′ untranslated region to enhance expression; an intron element to further enhance expression in certain cell types, such as monocot cells; a multiple-cloning site to provide convenient restriction sites for inserting the exogenous nucleic acid-encoding sequence and other desired elements; and a 3′ untranslated region to provide for efficient termination of the expressed transcript. For particle bombardment or with protoplast transformation, the expression cassette can be an isolated linear fragment or may be part of a larger construct that might contain bacterial replication elements, bacterial selectable markers or other elements. The exogenous nucleic acid expression cassette may be physically linked to a marker cassette or may be mixed with a second nucleic acid molecule encoding a marker cassette. The marker cassette is comprised of necessary elements to express a visual or selectable marker that allows for efficient selection of transformed cells. In the case of Agrobacterium-mediated transformation, the expression cassette may be adjacent to or between flanking T-DNA borders and contained within a binary vector. In another embodiment, the expression cassette may be outside of the T-DNA. The presence of the expression cassette in a cell may be manipulated by positive or negative selection regime(s). Furthermore, a selectable marker cassette may also be within or adjacent to the same T-DNA borders or may be somewhere else within a second T-DNA on the binary vector (e.g., a 2 T-DNA system).

In another embodiment, cells that have been modified by an exogenous nucleic acid, either transiently or stably, are carried forward along with unmodified cells. The cells can be sub-divided into independent clonally derived lines or can be used to regenerate independently derived organisms. Individual plants or animals or clonal populations regenerated from such cells can be used to generate independently derived lines. At any of these stages a molecular assay can be employed to screen for cells, organisms or lines that have been modified. Cells, organisms or lines that have been modified continue to be propagated and unmodified cells, organisms or lines are discarded. In these embodiments, the presence of an active exogenous nucleic acid in a cell is essential to ensure the efficiency of the overall process.

### Expression Strategies

Promoters for transformation have been described in the art; thus, the disclosure provides, in certain embodiments, novel combinations of promoters and a sequence encoding an exogenous nucleic acid, to allow for specifically introducing a recombination event into endogenous DNA (i.e., a genome). In one embodiment, a constitutive promoter is cloned 5′ to a sequence encoding an exogenous nucleic acid, in order to constitutively express the exogenous nucleic acid in transformed cells. This may be desirable when the activity of the exogenous nucleic acid is low or the frequency of finding and recombining the target site is low. It may also be desirable when a promoter for a specific cell type, such as the germ line, is not known for a given species of interest.

In another embodiment, an inducible promoter can be used to turn on expression of the exogenous nucleic acid under certain conditions. For example, a cold shock promoter cloned upstream of an exogenous nucleic acid might be used to induce the exogenous nucleic acid under cold temperatures. Other environmentally inducible promoters have been described and can be used in a novel combination with an exogenous nucleic acid-encoding sequence. Another type of inducible promoter is a chemically inducible promoter. Such promoters can be precisely activated by the application of a chemical inducer. Examples of chemical inducible promoters include the steroid inducible promoter and a quorum sensing promoter (see, e.g., You et al., 2006; U.S. Patent Application Publication No. 2005/0227285). Recently it has been shown that modified RNA molecules comprising a ligand specific aptamer and riboswitch can be used to chemically regulate the expression of a target gene (Tucker et al, 2005; International Publication No. WO2006073727). Such a riboregulator can be used to control the expression of an exogenous nucleic acid-encoding gene by the addition or elimination of a chemical ligand.

In other embodiments, the promoter may be a tissue specific promoter, a developmentally regulated promoter, or a cell cycle regulated promoter. Certain contemplated promoters include ones that only express in the germline or reproductive cells, among others. Such developmentally regulated promoters have the advantage of limiting the expression of the exogenous nucleic acid to only those cells in which DNA is inherited in subsequent generations. Therefore, a genetic modification by an exogenous nucleic acid (i.e., genetic recombination) is limited only to cells that are involved in transmitting their genome from one generation to the next. This might be useful if broader expression of the exogenous nucleic acid were genotoxic or had other unwanted effects.

Another contemplated promoter is a promoter that directs developmentally regulated expression limited to reproductive cells just before or during meiosis. Such a promoter has the advantage of expressing the exogenous nucleic acid only in cells that have the potential to pass on their genome to a subsequent generation. Examples of such promoters include the promoters of genes encoding DNA ligases, recombinases, and replicases, among others.

In addition to promoters, this disclosure provides for 5′ untranslated regions, introns and 3′ untranslated regions that can be uniquely combined with a exogenous nucleic acid-encoding sequence to create novel expression cassettes with utility for genome engineering.

### Detection of Exogenous Nucleic Acids in Recipient Cells

The disclosure also provides molecular assays for detecting and characterizing cells that have been modified as described herein. These assays include but are not limited to genotyping reactions, a PCR assay, a sequencing reaction or other molecular assay. Design and synthesis of nucleic acid primers useful for such assays, for instance to assay for the occurrence of a recombination event, are also contemplated.

Genotyping of cells may be performed on any cells or tissue as appropriate with the disclosure, including callus cells or tissue. The genotype of callus derived from transformed plant embryos can be determined by, for example, PCR analysis, using PCR amplification of the PPR10 gene.

Chloroplasts, among other plastids, are believed to have originated from bacteria and as such have retained some of the bacterial gene expression characteristics. For example, chloroplasts of land plants have polycistronic transcription units that resemble bacterial operons. In addition, chloroplast ribosomes are similar in protein content and antibiotic sensitivities to bacterial ribosomes. Chloroplasts also have bacterial-type RNA polymerases for chloroplast transcription, and ribonucleases that are derived from those in bacteria are involved in and processing of polycistronic primary transcripts to generate complex transcript populations and chloroplast RNA turnover.

Chloroplasts also exhibit similarities to eukaryotic gene expression, including the presence of introns, and modification of mRNA sequences by RNA editing.

Plastids regulate protein accumulation via translational control. For photosynthetic genes, for example, transcription is constitutive but the protein product is translated only in green tissues in the light. Translational control sequences can derive the same plastid gene as the promoter or a different plastid gene to create a chimeric promoter/leader construct that combines ideal functions. For example, a strong promoter may be combined with a leader sequence that directs translation across both light- and dark-grown tissues. An example of such a leader sequence is derived from the plastid clpP gene (Zhang 2012)). A bacterial-derived translational control sequence, such as in the bacteriophage gene 10 leader sequence (G10L), can also be used to direct high-level constitutive expression of multiple transgenes in plastids (Ye et al., 2001)). For transcript stability, a 3′-UTR region is used. The 3′UTR terminates transcription via a stem/loop region that forms in the RNA independent of the genetic background. Therefore, a 3′UTR region from a homologous plastid gene, heterologous plastid gene or bacteria may be used. Plastid expression signals that are derived from a different plant species may have an advantage in that the reduced nucleotide sequence identity may reduce or eliminate the possibility of intragenic recombination.

In some embodiments, a selectable marker gene and gene(s)-of-interest as described herein may be expressed from gene expression elements that function in plant plastids. As most plastid genes are constitutively expressed, a plastid promoter may be chosen based on its relative strength. In most cases, plastid promoter sequences derive from the plastid genome of the same plant species. Plastids contain different promoter types; those recognized by the plastid-encoded RNA polymerase (PEP), the nuclear-encoded RNA polymerase (NEP) or both. PEP promoter elements resemble the bacterial-like-10 and -35 recognition elements whereas NEP promoters have a single core promoter element. Plastid genes with NEP promoter typically are over-expressed in undeveloped plastid types, while plastid genes with PEP promoter elements are typically over-expressed in developed chloroplasts. Plastid genes with both PEP and NEP promoters are highly transcribed in both tissue types. An example of a NEP promoter active in non-green tissues is derived from the clpP gene. A strong constitutive promoter with both NEP and PEP elements is derived from the 16SrDNA gene (Prrn).

The disclosure further provides a kit comprising a single-use container comprising a callus or seed produced from a plant part as described herein. In some embodiments, it may be desirable to provide a plant part and reagents for producing callus tissue. In such a case, sterile reagents and tissue may be provided as appropriate. A kit may further comprise reagents for cell transformation, cell culture, or both.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, cell transformation, tissue culture, genetic complementation assays, or any assay useful in accordance with the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, media components, such as salts including MgCl₂, a polymerase enzyme, and deoxyribonucleotides, and the like, reagents for DNA isolation, or the like, as described herein. Such reagents or components are well known in the art. Where appropriate, reagents included with such a kit may be provided either in the same container or media as a primer pair or multiple primer pairs, or may alternatively be placed in a second or additional distinct container into which an additional composition or reagents may be placed and suitably aliquoted. Alternatively, reagents may be provided in a single container means. A kit of the disclosure may also include instructions for use, including storage requirements for individual components as appropriate.

### IX. Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR § 1.822 is used.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, “homoplasmic” refers to a eukaryotic plant cell whose copies of plastid DNA are all identical. Homoplasmic plastid DNA copies may be normal or mutated. In the case; however, that a mixed population of plastid DNA molecules exist, these are termed heteroplasmic, i.e., only occurring in some copies of plastid DNA. Heteroplasmic plastid DNA could arise, for example, when some copies of plastid DNA are mutated and some copies of plastid DNA are wild-type. Homoplasmy may occur naturally or otherwise.

As used herein, “non-photosynthetic” refers to a plant that is incapable of performing photosynthesis. Non-photosynthetic plants may be the result of a genetic mutation, whether natural or induced. A non-photosynthetic plant of the disclosure may be the result of a mutation in a chloroplast photosynthesis gene, or a nuclear gene that is involved with photosynthesis. One or more genes may be involved.

As used herein, “callus” refers to growing mass of unorganized plant cells. Callus culture is known in the art, and formation of callus tissue may be performed under sterile tissue culture conditions using reagents as appropriate for the particular application. Type I callus (less differentiated) and Type II callus (more differentiated) may grow on different types of media and at different rates. Individual embryos were placed onto medium in Petri plates in a grid pattern and grown in the dark at 28° C.

As used herein, a “chloroplast transit peptide” or “CTP” refers to a transit peptide that, when fused to a protein, acts to transport that protein into the chloroplast of a plant. Once inside the chloroplast, the transit peptide is cleaved from the protein, and the protein is free to perform its intended function. A nucleic acid encoding a CTP sequence can be operably linked to the nucleic acid sequence encoding a gene of interest to be targeted to the chloroplast. In accordance with the disclosure, a CTP from any appropriate plant species may be used as described herein.

As used herein, “domain” refers to a polypeptide that includes an amino acid sequence of an entire polypeptide or a functional portion of a polypeptide. Certain functional subsequences are known, and if they are not known, can be determined by truncating a known sequence and determining whether the truncated sequence yields a functional polypeptide.

As used herein, “expression construct” refers to a DNA construct that includes an encoded exogenous nucleic acid protein that can be transcribed.

As used herein, “exogenous DNA sequence” refers to a DNA sequence that originates outside the host cell. Such a DNA sequence can be obtained from a different species, or the same species, as that of the cell into which it is being delivered.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any article (e.g., plant, plant part such as a seed, or plant cell; gene or protein), composition, or device that “comprises,” “has” or “includes” one or more examples or features is not limited to possessing only those one or more examples or features and can cover other unlisted examples or features.

All methods described herein can be performed in any suitable order unless otherwise indicated herein or otherwise clearly contradicted by context. In addition, embodiments described herein in reference to complementation of chloroplast-encoded genes may also be appropriate for complementation of mutated nuclear genes encoding chloroplast-localized proteins involved in photosynthesis, and are therefore included in all embodiments as appropriate. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Embodiments

1. A method of expressing an agronomically beneficial trait in a plant plastid comprising: (a) expressing an exogenous nucleic acid in the plant nucleus, wherein the exogenous nucleic acid comprises sequences encoding i) a promoter functional in plants; ii) a chloroplast transit peptide; and iii) an enzyme capable of creating a double-stranded break in a plastid genome; OR a substitution mutation (b) selecting a recipient mutant plant or callus having non-photosynthetic chloroplasts; (c) growing a callus of the selected recipient mutant plant in culture; (d) transforming the mutant plant-line callus with a plastid transformation vector comprising a wild-type copy of the mutated chloroplast gene and one or more agronomically beneficial trait genes; and (e) selecting green, photosynthetic callus in the light; (f) regenerating a green plant carrying photosynthetic chloroplasts and the agronomically beneficial trait.

2. The method of embodiment 1, wherein the mutant plant-line callus is grown in light conditions.

3. The method of embodiment 1, wherein the mutant plant-line callus is grown in dark conditions.

4. The method of embodiment 1, wherein the promoter driving the exogenous nucleic acid in the nucleus is a constitutive promoter, a seed-specific promoter or an embryo-specific promoter.

5. The method of embodiment 1, wherein the plastid transformation vector comprises a chloroplast transformation vector.

6. The method of embodiment 1, wherein the plant comprises a corn plant or a soy plant.

7. The method of embodiment 1, wherein the non-photosynthetic homoplasmic chloroplast mutant plant line comprises a mutation in a chloroplast photosynthetic gene.

8. The method of embodiment 7, wherein the mutation in a chloroplast gene confers a non-green phenotype when grown under light conditions.

9. A chloroplast-transformed plant generated by the method of embodiment 1.

10. A plant part of the plant of embodiment 9, selected from the group consisting of a seed, stem, callus, meristem, embryo, leaf, and root.

11. A seed produced by the plant of embodiment 10.

12. A method for modification of one or more chloroplast genes in a plant comprising expressing in a plant nucleus an exogenous nucleic acid linked to a chloroplast transit peptide.

13. The method of embodiment 12, wherein expression of the exogenous nucleic acid results in a deletion mutant plant having at least one non-photosynthetic plastid.

14. The method of embodiment 13, further comprising transforming the at least one non-photosynthetic plastid with an expression construct comprising a wild-type copy of the mutant gene.

15. A non-photosynthetic chloroplast produced by the method of embodiment 13.

16. A kit comprising: a single-use container comprising a callus or seed produced from the plant part of embodiment 10.

17. The kit of embodiment 16, further comprising reagents for transformation, cell culture, or both.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1: Creation of Plastid Deletion Mutant Lines

### Generation of Chloroplast Deletion Mutant Lines Using Chloroplast Targeted Site-Specific Nucleases

Standard nuclear transformation can be performed using Agrobacterium-mediated nuclear transformation or particle bombardment. In either case, a selectable marker and the engineered enzyme transgene(s) were first cloned into a transforming plasmid. A plethora of nuclear transformation vectors are present in the art. A selectable marker is selected, which is different from the marker used for plastid transformation. The selectable marker, such as BAR or HYG, will be different from that subsequently used for plastid transformation.

The selectable marker can be driven by a plethora of common nuclear transgene expression elements as can be the exogenous nucleic acid. For the selectable marker, a constitutive promoter can be used.

For the exogenous nucleic acid, a constitutive promoter may be used. However, for the present disclosure, cutting of the chloroplast sequence that results in a mutant phenotype may prevent normal growth of TO transgenic plants. In certain embodiments, the promoter used for expression of the engineered nuclease (e.g., TALEN, Zinc Finger, or restriction endonuclease) is embryo-specific. In certain embodiments, an embryo-specific promoter can be advantageous because the relatively low copy number of the plastid genome in embryo-derived plastids will facilitate complete digestion of the genome molecules by the engineered nuclease, and so that the mutant phenotype, expected to be pale-green or albino, can be recovered in embryos from ears of the TO transformed lines without affecting vegetative growth of those plants. Callus from pale-green or albino mutants can then be subsequently maintained in sterile tissue culture as described above. Embryo-specific promoters include the maize C1-1B promoter (Li et al, 2015) and several uncharacterized genes reported recently (Liu et al; 2014). Additional promoters with embryo-enhanced expression that can be used including the maize and rice ubiquitin 1 promoters.

Plastid transit peptides that are expected to be efficient in embryo-derived plastids can come from a multitude of nuclear genes, including constitutively expressed genes and genes whose gene products naturally accumulate in those tissues. Examples of useful plastid transit peptides include;

(From WO 2008105890 A2) TaWaxy CTP (SEQ ID NO:1: Triticum aestivum granule-bound starch synthase CTP synthetic, codon optimized for corn expression: Clark et al., 1991) OsWaxy CTP (SEQ ID NO:2, Oryza sativa starch synthase CTP; Okagaki, 1992). Other plastid transit peptides that enable targeting in endosperm include the rice FtsZ gene, maize non-photosynthetic ferrodoxin III gene and the small subunit of Rubisco (Primavesi et al., 2008). Another example is a synthetic version of the maize EPSP Synthase CTP (Preuss et. al., 2012).

A TALEN nuclease contains the DNA-binding domain from the TAL-effector of Xanthomonas fused to a DNA cleavage domain usually derived from the FokI restriction endonuclease. The DNA binding domain contains a repeated highly conserved 33-34 amino acid sequence with divergent 12th and 13th amino acids. These two positions, referred to as the Repeat Variable Diresidue (RVD), show a strong correlation with specific nucleotide recognition. This straightforward relationship between amino acid sequence and DNA recognition has allowed for the engineering of specific DNA-binding domains by selecting a combination of repeat segments containing the appropriate RVDs. Several software programs, for example, TALEN Targeter, can be used to design a series of TALE repeats to recognize any chloroplast DNA sequence of interest.

To be effective in double-strand breakage, two TALENs may need to be designed that recognize DNA sequences of the opposite strands of the genome. Target sites for these TALENs are typically within 25 nucleotides of each other but can be much further apart. In some cases, a new compact TALEN (cTALEN) (Beurdeley M. 2013) made of only a single polypeptide can be used to simplify cloning and expression of the nuclease.

A zinc-finger nuclease, carrying zinc-finger DNA binding domains fused to a FokI DNA cleavage domain may alternatively be used to create deletions in the chloroplast genome. Each 30 amino acid zinc finger primarily binds to a triplet within the DNA substrate. Binding to longer recognition sequences can be achieved by linking several zinc-finger domains together.

The TALEN(s) or Zinc-finger nucleases are fused in frame to an N-terminally located chloroplast transit peptide, as described above.

Selection of chloroplast genome sequences targeted for deletion are show in previously presented Table 1. Deletions of any sequences in these genes required for photosynthesis would result in the non-photosynthetic phenotype and plant cells comprising plastids comprising such deletions could be used as recipient for plastid transformation.

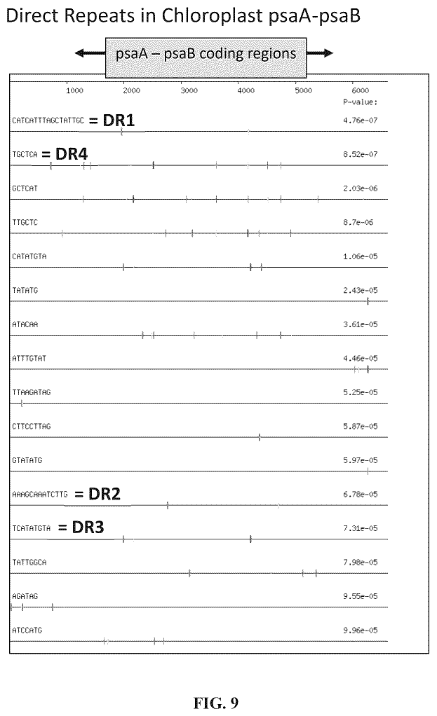

TALEN cut sites in a psaA/B gene region are identified and will be targeted for cleavage by suitably engineered TALENS. An ˜6 kb genomic region encompassing the psaA/B gene region was searched for the presence of repeated sequences that may help to catalyze ectopic recombination and facilitate deletion formation in that region of the genome, though deletions may be larger or smaller than this. The RepFind software package (Betley et al., 2002; on the https internet site “zlab.bu.edu/repfind/form.html”) was utilized for this analysis. Minimum direct repeat size was set at 6 nucleotides, though shorter repeat sizes may also catalyze recombination, as described above. A graphic representation of the locations of the direct repeat sequences along with their positions in this region are shown in FIG. 8. CTP-TALENs are created that recognize sequences and create a double strand break in the vicinity of the psaA/B region directly repeated sequences. Potential cleavage sites are shown in FIG. 6 relative to the location of the direct repeats. FIG. 9 shows the sequence and locations of numerous directly repeated sequences of varying lengths in the psaA/B genomic region. In certain cases, chloroplast mutant lines will be created after expression in the nucleus of a chloroplast-targeted restriction endonuclease. The AscI enzyme amino acid sequence from Arthrobacter was retrieved from the public database (uniport.org). A maize codon-optimized gene was created using IDT (IDTDNA.com) software tool. The maize EPSPS chloroplast transit peptide, CTP2, was translationally fused to the N-terminus of AscI to create the CTP-AscI gene. The nucleotide and amino acid sequences are: Arthrobacter AscI amino acid sequence:

Uniprot: E3VXA3
(Seq ID 58)
MIEFPEYRDSSAAPKISDLERLMDRSLTNSQYVDGANAARLLGTFIRSF
RSVIGSAEESATRANLVEAHDEAKLFGLMLSAGFDLICNAEYVHGRLVN
NKWIYCHRGGEPAVAYYSFLKQCPRCCLDRGLEGRLSGAQHKPTSHHIG
EITTVAIALLLQLVAAANENPFEIATITKQSHDVDAIGFRDDLLVLFEI
KASPMVSFPLVTELEEPMLQEGPDGPVEYRQHSLVDLTLQGREFAVAIP
HAETAIPLGEREGESWPYEPLIDYFSVPANAASYLQAWIELYAAYRTPK
TQRAGRTAALAYLVNGWGDEIDSNKTKPGLGRTDDVKKGTYQLLKFGSY
YRDDAASVPVRGALVANLDPLFLRPGYIDGLSDVRWGHGRDFTLEEGEY
RIAEGSLRHLYDAILAFNDPLLNDPLLQEIFDLGAVERKLANGDLEALL
DKWIARPEIVLDPS
CTP-AscI nucleotide sequence from pPTS150 (CTP2
in bold, maize codon-optimized AscI in plain
text)
(Seq ID 59)
ATGGCGCAGGTCTCCCGCATTTGTAATGGTGTGCAGAATCCTTCGCTCA
TCAGCAATTTGTCCAAAAGCTCCCAAAGAAAGTCGCCTCTCAGCGTTTC
ATTGAAAACGCAGCAGCATCCCCGCGCCTACCCGATTTCTTCTTCATGG
GGACTTAAAAAGTCGGGCATGACACTCATAGGCTCCGAACTTCGGCCAC
TCAAAGTGATGTCTTCCGTTTCAACGGCGTGTATGCTGATGATTGAATT
TCCAGAGTATAGGGATTCATCTGCGGCGCCGAAAATAAGCGACCTTGAA
AGGCTGATGGATAGGTCACTCACTAATTCGCAGTACGTGGATGGAGCCA
ATGCTGCGCGCCTGTTGGGCACTTTTATTCGGTCGTTTCGCTCTGTTAT
CGGGTCTGCAGAAGAGTCAGCTACCAGGGCGAATCTCGTGGAGGCACAC
GACGAGGCTAAACTGTTTGGCCTGATGTTGTCTGCAGGGTTCGACCTCA
TTTGCAATGCCGAATATGTTCATGGACGCCTGGTCAACAACAAATGGAT
ATACTGCCACAGAGGCGGCGAACCAGCAGTCGCCTACTATTCATTCCTG
AAGCAATGTCCGCGGTGTTGTCTCGATAGAGGTTTGGAAGGAAGACTGT
CCGGCGCACAACATAAACCAACATCTCATCACATTGGTGAAATCACTAC
AGTGGCAATTGCTCTCCTGTTGCAGCTGGTTGCCGCGGCAAACGAAAAC
CCTTTCGAAATAGCGACGATAACGAAGCAATCGCACGACGTCGACGCAA
TCGGATTTAGAGATGACCTTTTGGTTCTCTTTGAAATCAAGGCTTCTCC
GATGGTTAGCTTTCCTCTTGTTACGGAACTTGAAGAGCCCATGCTCCAA
GAGGGGCCTGATGGCCCTGTCGAATATCGGCAACATTCCCTGGTCGACC
TCACGCTCCAAGGACGCGAGTTCGCCGTCGCTATACCGCACGCGGAGAC
GGCAATCCCGCTTGGAGAAAGGGAGGGAGAAAGCTGGCCGTACGAACCG
CTCATCGACTATTTTTCGGTTCCAGCTAATGCTGCCTCATACCTCCAGG
CTTGGATCGAGCTTTACGCTGCGTACCGGACCCCCAAAACTCAACGCGC
AGGTCGGACGGCCGCTCTTGCCTACCTTGTGAACGGGTGGGGTGACGAG
ATCGATTCAAACAAAACGAAGCCCGGACTCGGCAGAACGGATGATGTCA
AGAAGGGAACATATCAGCTGCTTAAATTTGGAAGCTACTACCGCGATGA
TGCCGCTTCAGTGCCGGTGAGGGGGGCACTCGTGGCTAACTTGGACCCG
CTTTTTCTTCGGCCCGGTTACATAGATGGGCTTTCTGATGTGAGATGGG
GGCACGGCAGAGACTTCACCCTGGAAGAGGGAGAATATAGGATTGCCGA
AGGCTCTCTGCGGCACTTGTATGATGCAATCTTGGCTTTTAATGATCCA
CTCCTCAACGATCCGTTGCTCCAAGAAATATTCGATCTCGGCGCAGTTG
AACGGAAATTGGCAAACGGCGACCTCGAGGCGCTGCTCGATAAGTGGAT
TGCGCGCCCTGAGATTGTTCTGGACCCATCCCCTTATGATGTTCCTGAT
TATGCTtaa
CTP-AscI amino acid sequence from pPTS150 (CTP2
in bold)
SEQ ID 60
MAQVSRICNGVQNPSLISNLSKSSQRKSPLSVSLKTQQHPRAYPISSSW
GLKKSGMTLIGSELRPLKVMSSVSTACMLMIEFPEYRDSSAAPKISDLE
RLMDRSLTNSQYVDGANAARLLGTFIRSFRSVIGSAEESATRANLVEAH
DEAKLFGLMLSAGFDLICNAEYVHGRLVNNKWIYCHRGGEPAVAYYSFL
KQCPRCCLDRGLEGRLSGAQHKPTSHHIGEITTVAIALLLQLVAAANEN
PFEIATITKQSHDVDAIGFRDDLLVLFEIKASPMVSFPLVTELEEPMLQ
EGPDGPVEYRQHSLVDLTLQGREFAVAIPHAETAIPLGEREGESWPYEP
LIDYFSVPANAASYLQAWIELYAAYRTPKTQRAGRTAALAYLVNGWGDE
IDSNKTKPGLGRTDDVKKGTYQLLKFGSYYRDDAASVPVRGALVANLDP
LFLRPGYIDGLSDVRWGHGRDFTLEEGEYRIAEGSLRHLYDAILAFNDP
LLNDPLLQEIFDLGAVERKLANGDLEALLDKWIARPEIVLDPS

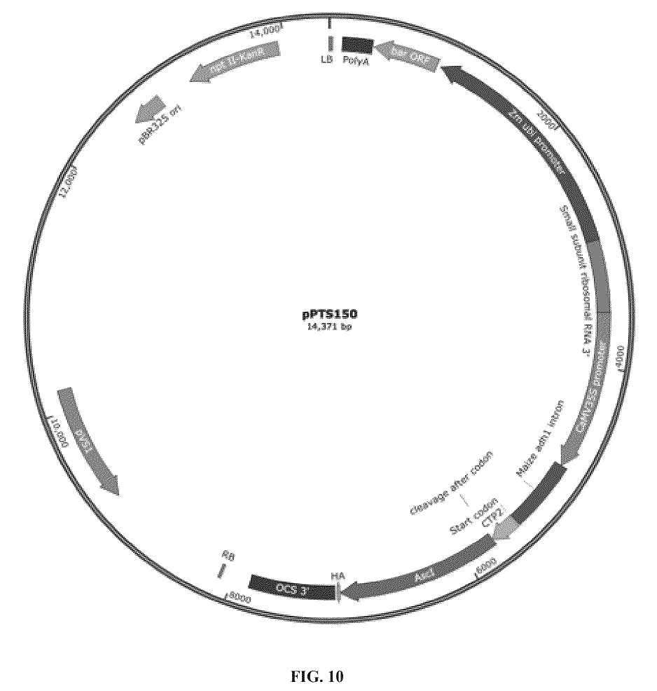

The CTP-AscI gene was cloned next to a 3X CaMV promoter and the maize AdhI gene intron to enhance translation of the gene. This transgene is cloned into a vector carrying a selectable bar gene driven by the maize ubiquitin 1 promoter. The T-DNA right and left border regions are included for Agrobacterium-mediated transformation. The resultant plasmid, pPTS150, is shown in FIG. 10.

The gene sequence of the Flexibacter FspA1 restriction enzyme is not available in the public databases. However, purified enzyme can be purchased from ThermoFischer (catalog number ER1661) and the amino acid sequence deduced using standard protocols of Edman degradation and/or mass spectrometry. Briefly, the enzyme can first be digested into smaller peptides using endopeptidases trypsin or pepsin. Peptides can be sequenced via Edman degradation that produces an ordered amino acid sequence from the N-terminus, or via MALDI-TOF mass spectrometry that results in a specific mass used to derive the amino acid sequence. The deduced amino acid sequence is then used to search protein and nucleic acid databases to find candidate gene sequences for cloning, or the entire gene encoding the protein can be synthesized directly. Once obtained, the gene sequence may be modified as above to include a chloroplast transit peptide, and cloned into a nuclear transgene expression vector suitable for creating transgenic plants.

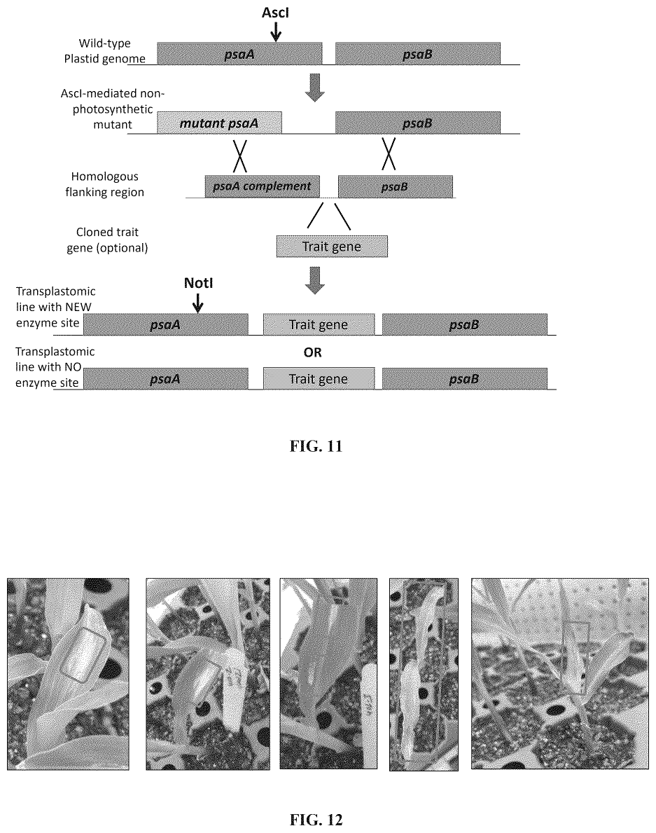

FIG. 11 illustrates complementation of a non-photosynthetic mutant that arises from AscI-mediated deletion in the chloroplast psaA gene. The wild-type complementing psaA gene sequences may be used. Alternatively, the psaA gene sequence may be modified to eliminate the original AscI site or to create a novel restriction endonuclease site that does not occur in wild-type populations of maize. Removal of the wild-type restriction endonuclease site or incorporation of a new restriction endonuclease site can be designed to not alter the wild-type amino acid sequence of the protein, or if necessary, may be designed to create conservation amino acid substitutions. In either case, selection of transformants is still via photosynthetic complementation and may or may not include an adjacent trait transgene.

### Example 2. Complementation of Chloroplast Deletion Mutants Via Nuclear Transgenesis and Subsequent Targeting of the Protein to the Chloroplast

Chloroplast deletion mutants created by TALENs or a restriction endonuclease represent a novel reagent for studying photosynthetic gene function. In some cases, it may be advantageous to complement these mutant lines via a nuclear-encoded transgene. Nuclear transgenesis methods are routine in the art and may be performed by standard Agrobacterium-or biolistic-based methods. Generally, the throughput of nuclear transformation is very high, enabling the study of large numbers of transgenes or permutations of the same transgene. Using this approach, the chloroplast photosynthetic gene may be relocated to the nucleus and subsequently delivered to chloroplasts via a chloroplast transit peptide. Kanevski and Maliga (1994) used this approach in tobacco; first creating a tobacco chloroplast deletion in rbcL via insertion of a selectable aadA and then subsequently performing nuclear transformation of that line to supply the RuBisCo large subunit via a chloroplast transit peptide. It should be noted that an efficient chloroplast transformation method already existed in tobacco, enabling the creation of the original deletion mutants, and no attempt was made with the non-photosynthetic mutant lines to engineer a better RuBisCo.

### Example 3: Plant Transformation to Add Agronomically or Non-Agronomically Beneficial Traits

Non-photosynthetic callus derived from chloroplast deletion mutants or nuclear non-photosynthetic mutants would be used as recipient for plastid transformation using the complementing sequences as described above. The plastids of the non-photosynthetic mutant plant are then transformed with a transformation construct having a nucleic acid encoding a functional copy of the gene that was inactivated. Also present on the transformation construct, adjacent to the complementing sequence, is a nucleic acid encoding at least one gene conferring a beneficial trait, such as resistance to a herbicide, such as the gene for EPSPS that confers resistance to glyphosate, or resistance to a pesticide, such as a crystal protein gene from Bacillus thuringiensis that produces an insecticidal protein. The plant is then regenerated from the callus tissue by altering the growth culture conditions to induce shoot formation. Once the plant has grown to a sufficient size, it is transferred into soil. The insecticidal protein would be expressed in the plant tissue such that ingestion of the plant tissue by the target pest would result in death of the pest. Herbicide resistance would be verified by spraying with a lethal dose of glyphosate. Verification of the newly added agronomically or non-agronomically beneficial trait is done using PCR with primers specific for the newly added nucleic acid. Any agronomically or non-agronomically beneficial trait may be added to a plant as described herein.

### Example 4. Use of a Restriction Enzyme that Cuts the Chloroplast Genome in Multiple Locations

Similar to above, in certain cases, chloroplast mutant lines will be created after expression in the nucleus of a chloroplast-targeted restriction endonuclease that cuts the chloroplast genome in multiple locations. For example, the restriction enzyme FspI recognizes 7 sites in the maize variety A188 chloroplast genome. Two of the recognition sites reside within the psaA/B locus; one site resides within the psaA gene while the other site resides in the psaB gene. Thus digestion of either or both sites could result in the non-photosynthetic phenotype. Further, an FspI site resides within the psbB gene encoding the Photosystem II CP47, and another site in the atpB gene encoding the B-subunit of the chloroplast ATP synthase enzyme, mutation in either of which would result in the non-photosynthetic phenotype. Sugimoto et. al (2020) used Arabidopsis stable or transient nuclear transformation to over-express and target various restriction enzymes that have multiple recognition sites in the chloroplast genome. The resulting transgenic plants showed leaf variegation associated with impaired chloroplast function, and in some cases transgenic lines exhibited rearrangements in the chloroplast genome wherein other cases only small-scale changes in the chloroplast genome occurred. There was no effort to further culture these variegated mutants.

The FspI gene from Fischerella musicola (NCBI Reference Sequence WP_016862289.1) was synthesized using maize nuclear optimized codons, cloned downstream of the CTP2 chloroplast transit peptide, and expressed from the enhanced CaMV 35S promoter or Maize Ubiquitin 1 promoter, to create the CTP2_FspI gene, as described for the pPTS150 plasmid above and in FIG. 10. The amino acid and nucleotide sequences of FspI are as follows:

SEQ ID 9: FspI amino acid sequence (CTP2 transit
peptide not shown)
MLTNNEIERLRQAIIATIASPVIGSIEDYTWEAIFHYVKDIPLSDPALG
RSKLLYDAVDVVTKTGWSLKSLQLKSLNFKSPFLFVIQRADILKKSVQL
GFPGLTEQSSPDELGAAIIQHWNEKIILSQAAQSVVNSYEGILLKTIKG
YEYIYCEFPLDPLDPSTFSWAWTVDKTTGGAGVGLQGSIVGKTELVWYK
NQKQLFRARTIPAQAVRITVERTRLTLDRYVKTVIFALQDQINMQFSEN
EPEEPYDVPDYA*
SEQ ID 10: Maize codon optimized FspI coding
region (CTP2 transit peptide not shown)
ATGCTGACCAATAATGAAATAGAAAGGCTGAGACAGGCGATCATTGCCA
CCATCGCATCGCCTGTCATTGGTTCGATTGAGGACTATACTTGGGAGGC
TATCTTTCACTATGTCAAAGATATACCTCTCTCTGATCCAGCTCTCGGC
AGAAGCAAACTCCTCTATGACGCTGTCGACGTGGTTACGAAGACGGGTT
GGTCGCTGAAGTCTCTTCAGCTCAAGTCCTTGAACTTCAAATCTCCTTT
TCTCTTTGTCATACAAAGGGCTGATATTCTGAAAAAATCCGTCCAACTG
GGATTCCCAGGTCTGACAGAGCAAAGCAGCCCAGATGAACTCGGTGCTG
CGATAATACAACATTGGAATGAAAAGATTATACTCTCGCAAGCCGCGCA
GTCCGTGGTTAACAGCTATGAAGGGATTCTTCTCAAAACAATAAAGGGT
TACGAGTACATTTATTGCGAGTTCCCTCTcGATCCTCTGGACCCAAGCA
CATTTTCGTGGGCTTGGACCGTGGACAAGACGACGGGGGGTGCGGGAGT
TGGTTTGCAGGGATCAATCGTTGGAAAGACGGAGCTGGTTTGGTACAAG
AACCAGAAGCAACTTTTTCGGGCGAGGACCATTCCTGCACAAGCCGTTA
GAATAACGGTGGAACGCACCAGGCTGACGCTCGACAGGTACGTTAAAAC
AGTGATCTTTGCCTTGCAGGATCAGATCAATATGCAATTTTCGGAGAAC
GAGCCGGAAGAGCCTTATGATGTTCCTGATTATGCTtaa

### Example 5: Screening for Pigment Mutants in Transgenic Maize Lines Expressing Chloroplast-Targeted Restriction Enzymes

Stable maize transgenic plants lines were generated via Agrobacterium-mediated transformation of chloroplast-targeted restriction enzyme vectors. In the first experiment, regenerated TO stable transgenic plant lines had no obvious mutant phenotypes, so plants were grown to maturity in the greenhouse to collect seeds and look for mutant phenotypes in the T1 generation. T1 seeds were sown in soil in a growth chamber and seedlings were monitored for albino leaves or leaf sectors. Several seedlings from 2 independent T0 transgenic lines that carry that chloroplast targeted AscI restriction enzyme were observed to have yellow or albino leaf sectors (examples show in red boxes in FIG. 12). No albino sectors were observed on numerous wild-type seedlings that were sown at the same time, indicating the transgenic albino sectors are true mutants. Thus, the albino sectors represent candidate chloroplast mutants.

### Example 6 Use of a Chloroplast Targeted I-CreII Homing Endonuclease to Disrupt the Chloroplast psbA Photosynthetic Gene

Similar to transgenic expression of the chloroplast targeted restriction enzymes above, and described in above, a I-CreII homing endonuclease was codon optimized for maize, cloned downstream of the chloroplast transit peptide and overexpressed in transgenic plants from the maize ubiquitin 2 promoter (pPTS410). The I-CreII homing endonuclease recognition sequence is encoded in a conserved sequence of the chloroplast psbA photosynthetic gene, and endonuclease digestion creates a 2-nucleotide overhang that can be repaired via non-homologous end joining to create a frameshift mutation or other lesion in the coding region to create a nonphotosynthetic mutant. The region around the I-CreII recognition sequence (staggered cut site in bold) that is conserved across monocots, maize (Zm; SEQ ID 61), rice (Os; SEQ ID 62) and sorghum (Sb; SEQ ID 63) is shown below.

| | |
|---|---|
| Sb I-CreII | ATATTGGAAGATTAATCGACCGAAATAACCGTGAGCAGCCACAATATTATAAGTCTCTTC |
| Zm I-CreII | -TATTGGAAGATTAATCGACCAAAATAACCGTGAGCAGCCACAATATTATAAGTCTCTTC |
| Os I-CreII | -TATTGGAAGATTAATCGGCCAAAATAACCATGAGCGGCCACAATATTATAAGTTTCTTC |
| | ***************** ** ******** ***** ***************** ***** |

SEQ ID 11: Maize codon-optimized amino acid sequence (derived from
Chlamydomonas reinhardtii)
MTTKKTIQFYANIAQVRNHELISLSNIQTPSQGSITIFCKTCKTSFTTTARSYQNARKTGCPQCKA
KTTSENWKGKIRTKSPEEASKQAVLNEYKQQKHLQKGLAYAHISNKEDLKIFLKESPNVYNNFI
LQRIDHPVIGKYTENHHIIPKHTGGPHKRWNLIKLTPEDHMEAHRLRALVYNEAGDHQAIRFRT
NPSELVERRLRGNQIANETRLRERTGIYAEGASSKGGRIGGLVKSHEKDLKQSTKMSQPVLKAL
YEGSRWKHLQSGTELNLQPNRLFTLPQLVQKLLEALPPCKDKEVLERAKTSTVTSNLARVIKKQ
RPSAYGWITFPYDVPDYA*
SEQ ID 12: Maize codon-optimized Nucleotide sequence
ATGACGACAAAGAAAACAATCCAGTTCTACGCTAATATCGCGCAAGTGCGCAACCACGAGC
TTATTTCTTTGTCAAACATTCAGACCCCGAGCCAAGGTAGCATCACAATTTTCTGTAAGACTT
GCAAAACATCATTCACGACTACGGCTAGGTCTTATCAGAACGCACGGAAAACTGGGTGCCC
TCAGTGCAAGGCTAAGACAACCTCGGAAAACTGGAAAGGTAAAATACGGACGAAATCCCCT
GAAGAGGCCTCGAAGCAGGCAGTGCTCAATGAGTATAAACAACAAAAACATTTGCAAAAG
GGTCTTGCCTATGCCCACATTTCAAATAAAGAAGATCTGAAAATCTTTTTGAAGGAATCGCC
GAATGTCTATAACAATTTTATACTTCAGAGAATCGATCATCCGGTGATTGGGAAATATACAG
AGAACCACCACATTATCCCAAAACACACTGGTGGGCCGCACAAAAGGTGGAATCTGATTAA
ATTGACGCCGGAGGACCATATGGAAGCGCATCGCCTTAGGGCACTCGTGTATAATGAAGCG
GGCGACCATCAGGCAATACGCTTCAGGACCAATCCTAGCGAGCTGGTTGAGCGGAGGCTTA
GAGGAAACCAAATCGCTAACGAGACGAGACTCAGGGAGAGGACGGGTATTTATGCGGAGG
GGGCATCATCCAAGGGTGGGAGAATAGGTGGACTGGTCAAATCTCACGAGAAAGACCTGAA
ACAAAGCACCAAAATGAGCCAGCCCGTTCTGAAAGCGTTGTACGAAGGCTCGAGATGGAAG
CACCTTCAATCCGGCACAGAGCTTAACCTGCAACCCAACCGGCTCTTTACTCTCCCCCAGCT
CGTGCAGAAGCTGTTGGAAGCTCTTCCGCCCTGCAAAGATAAAGAGGTCCTGGAACGGGCA
AAGACTTCAACTGTGACCTCCAATCTGGCTCGCGTGATCAAAAAACAGAGGCCTAGCGCCT
ATGGATGGATTACGTTCCCTTATGATGTTCCTGATTATGCTTAA

### Example 7: Inducing Chloroplast Mutations in Monocots Leaf, Facilitated by Use of BBM and WUS Transcription Factors

As described in above, overexpression of Babyboom (BBM) and Wuschel (WUS) transcription factors in maize and other monocots can induce embryogenesis from multiple tissues, including leaf tissues, that normally would not be embryogenic and thus not used for plant transformation. Over-expression of BBM and WUS can be via stable nuclear transgenesis or via transient expression in the target tissue of choice. BBM and WUS gene expression can be controlled by a variety of transgene expression elements, though expression in leaf cells is preferred using either the maize ubiquitin 1 promoter or the maize PLTP gene promoter for ectopic expression of the BBM gene, whereas expression of WUS is often controlled by the IN2 gene promoter (GenBank: MT221179.1), an enhanced IN2 gene promoter variant, or controlled by an auxin inducible promoter, termed axi1G (Lowe et. al. 2018). Overexpression of BBM and WUS using these promoter combinations is sufficient for rapid somatic embryo formation from leaf base carrying rapidly dividing young plastids and more mature leaf tissues carrying developed chloroplasts.

Stable transgenic plant lines carrying two examples of BBM and WUS overexpression constructs were created via Agrobacterium mediated transformation, PLTP: BBM+IN2: WUS and PLTP: BBM+Axi1G: WUS. Transgenic plants lines carrying PLTP: BBM+IN2: WUS have significant levels of embryogenesis potential in the basal portion of maize leaf sections when placed onto media carrying the appropriate plant growth hormones. Likewise, transgenic plant lines carrying PLTP: BBM+Axi1G: WUS have embryogenic potential throughout most of the leaf including the basal section and the more developed regions along the entire leaf surface.

Nuclear retransformation of PLTP: BBM+IN2: WUS and PLTP: BBM+Axi1G: WUS transgenic lines was performed using Agrobacterium-mediated transformation with T-DNA vectors carrying the chloroTALENs, restriction enzymes (pPTS150, pPTS191) and I-CreII homing endonuclease (pPTS410). Selection for bialaphos resistance encoded on the T-DNA was employed. Transgenic plant lines are regenerated under selection pressure to ensure growth only of transgenic embryogenic callus and subsequent regeneration into plant lines.

Retransformed transgenic plant lines can be either uniformly pale green or albino indicating potential homoplasmic chloroplast mutant lines, or they can be chimeric with both green and albino sectors in regenerated leaf sectors. Mutant leaf sectors would normally be difficult or impossible to recover, since wild-type maize leaf is not regenerable and sectored mutants may not be recovered in the germline. However, the presence of BBM and WUS in the genetic background enables recovery of pure mutant (albino or pale green) lines via regeneration of dissected mutant leaf sectors. Screening of retransformed lines will reveal albino or pale green sectors in some lines, that can be dissected and placed onto appropriate media with plant growth hormones to stimulate embryogenic callus formation from the mutant leaf sectors. Embryogenic callus that remains non-green in the light can be confirmed my molecular analysis to carry plastid mutations in the targeted gene. Mutant callus can be amplified in tissue culture and subsequently used for chloroplast retransformation as described above.

### Example 8. TALEN-Mediated Indel Mutations Result in the Non-Photosynthetic Mutant Phenotype

TALEN cut sites may be only from ˜15-25 nucleotides apart or may be much further apart, as described in above. In many cases, double-stranded break repair will result in small indels—a small deletion or insertion of a nucleotide or nucleotides at the cut site, that results in a frameshift mutation in the target gene coding region. In these cases, the frameshift mutation in the reading frame of the gene may result in a mutation that disrupts the function of the gene, resulting in a non-photosynthetic mutant.

### Example 9. Non-Photosynthetic Mutation Resulting from TALE-Deaminase Base Editing

Mutations in organelle genes can also be created via base substitution that creates a stop codon or other loss-of-function mutation in the target gene. Base substitution can be catalyzed by newly developed base editors, cytosine deaminase base editors (CBEs; catalyze C/G to T/A transition) or adenosine deaminase base editors (ABEs; catalyze A/T to G/C transition) that are fused to the DNA-binding function of CRISPR (reviewed in Vu et. al. 2019). More recently, base substitution in human (Mok et. al. 2020) and mice mitochondria (Lee et. al. 2021) or plant chloroplasts (Kim et. al., 2021 reported online https://doi.org/10.21203/rs.3.rs-145710/v1) has been shown using a TALE DNA binding domain fused to a newly identified cytosine base editor from Burkholderia cenocepacia, double-stranded DNA deaminase toxin A, termed DddA (Mok et. al. 2020), that uniquely catalyzes the deamination of cytidines on double-stranded DNAs, rather than previously characterized deaminases that work only on single-stranded DNA. Interestingly, DddA converts cytosine to uracil, which can be mutagenic itself due to the action of uracil DNA glycosylase (UDG) that initiates base excision repair through uracil removal. Engineered split DddA N- and C-terminal halves carrying the deaminase domain are inactive until brought together on target DNA by adjacently bound TALE DNA binding domain fusion proteins, that can then catalyze base editing on double-stranded DNA. Addition of an uracil glycosylase inhibitor protein (UGI) to the C-terminus of the fusion protein significantly enhanced the base editing efficiency of the TALE-DddA (Mok et al. 2020).

Addition of a mitochondrial targeting sequence was used to direct a mitoTALE-DddA-UGI fusion to edit multiple genes in the mitochondrial genome in HeLa cells (Mok et. al., 2020) or mice (Lee et. al. 2021). The mitoTALE-DddA-UGI fusions were used to target both silent mutations and mutations designed to mimic known human mitochondrial disease alleles. In one case, the TALE-DddA enzyme was used to create a premature stop codon within the coding region of the mitochondrial ND5 gene to investigate the effects of a loss of function mutation.

The TALE-DddA-UGI fusion enzymes were also used to edit chloroplast and mitochondrial genes in protoplasts derived from dicot plants, lettuce and rapeseed, using plant chloroplast or plant mitochondrial targeting peptides (Kim et al. 2021 reported online). Although plants were not regenerated, protoplasts and calli derived from protoplasts were shown to carry base edits. Chloroplast target genes included the psbA and psbB photosynthetic genes, but no attempt was shown to create a change-of-function in those genes.

A chloroplast-targeted TALE-DddA-UGI fusion enzyme could thus be used to target any of the chloroplast photosynthetic genes described in Tables I, II or III shown above. To create a non-photosynthetic mutant using the cytodine base editor approach, a premature stop codon within the coding region of the gene could be created, for example, by converting CAA, CAG or CGA to TAA, TAG or TGA stop codons, respectively. Several additional nucleotide transitions are possible to create any of the TAA, TAG or TGA stop codons by inspection of the nucleotide sequence of the target gene and mutation of the appropriate nucleotide.

### Example 10: Sequence Identity of Chloroplast Gene Target Sites Enables Cross-Crop Utility of Enzymes Involved in Creating Non-Photosynthetic Mutants

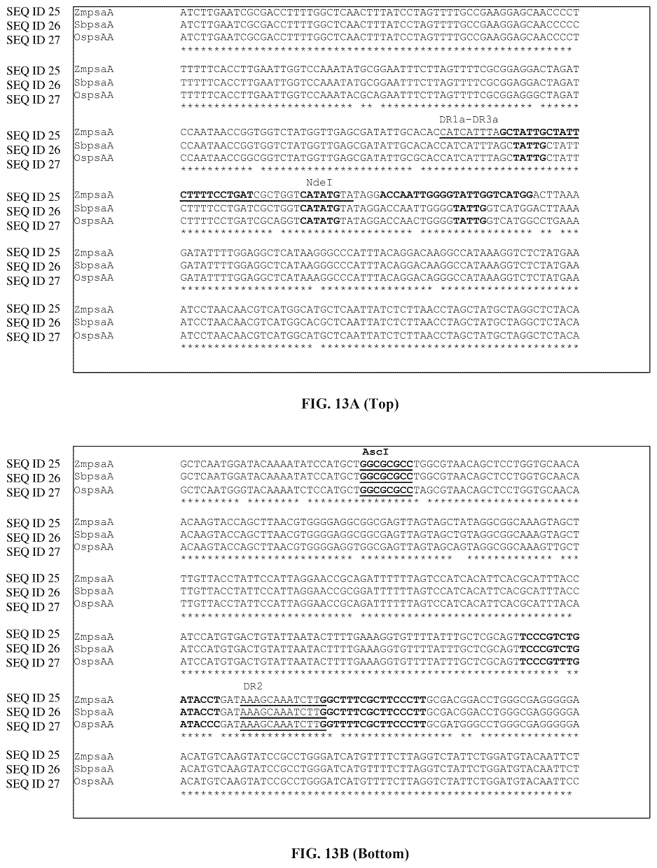

Chloroplast genomes are highly conserved in gene order and nucleotide sequence as described above. Within monocots and within dicot plant lineages, the nucleotide sequences of conserved photosynthetic gene coding regions can be as high as 97-100% identical. Therefore, TALEN enzymes that cause a double strand break or TALE-DddA base transition enzymes that are created to target specific nucleotide sequences in the chloroplast genome of one plant species may have a high likelihood of activity on the same or highly related sequence in the chloroplast genome of another closely related plant species. FIG. 13 illustrates representative examples of sequence conservation of chloroplast photosynthetic genes and resulting identity of the TALEN or restriction enzyme-mediated double-stranded break targeting sequences across species. For example, FIG. 13 (top and bottom) shows an example of sequence conserved TALEN target sequences (TALEN left half and right half shown in bold) in maize (ZmPsaA), sorghum (SbPsaA) and rice (OsPsaA), and the position of direct repeats sequences (underlined) that may be involved in homology dependent double strand break repair. In some cases, additional direct short repeats within the TALEN left and right half (TATTG in bold in FIG. 13 top) or a fortuitous restriction enzyme recognition sequence (NdeI) used for screening of genome sequence modifications with the spacer region between each TALEN half are shown. In another example (FIG. 13 bottom), the unique AscI restriction enzyme site that is targeted by the chloroplast-targeted AscI restriction enzyme is shown, conserved in the chloroplast genome of sall three monocot plant species.

### Example 11. Complementation of the Mutant Phenotype and Concurrent Co-Transformation of the Plastid Genome

As described in complementation of the mutation may be achieved using the wild-type copy of the mutant gene for selection, while transgene insertion may be directed to a different region(s) of the chloroplast genome. Cotransformation of the chloroplast genome using multiple independent plasmid vectors has been shown and can be greater than 50% efficient (Ye et. al. 2003). In this example, the mutant phenotype would be complemented by homologous insertion of a wild-type copy of the mutant gene or via gene conversion of the mutation with a wild-type copy of the gene located on one of the transforming plasmids. Transgenic trait gene insertion would be directed a different region of the chloroplast genome and not located adjacent to the gene encoding the original non-photosynthetic mutation.

In some cases, complementation of the photosynthetic mutation resulting in green photosynthetic cells may still be difficult to purify away from non-transformed mutant cells. Thus during subsequent plant regeneration, the plants may still be chimeric and transformed chloroplasts not homoplasmic. To facilitate purification of homoplasmic transformed chloroplasts in this case, it may be advantageous to utilize a second selectable marker such as an antibiotic or herbicide resistance gene, or a screenable marker (Khan and Maliga 1999) such as a fluorescent protein like GFP or PhiLOV. The cotransforming plasmid therefore may contain a trait gene(s) and may also carry the second selectable marker or screening marker for insertion into the chloroplast genome to help identify transformed chloroplasts and prevent regeneration of non-transformed cells. If a second selectable marker or screenable marker is utilized, it can subsequently be removed from the chloroplast genome by marker excision technology, which is also efficient in chloroplasts (Hajdukiewicz et. al. 2001; Corneille et. al. 2001; Lutz et. al. 2004).

### Example 12 ChloroTALEN Vector Design and Identification of Non-Photosynthetic Mutants

As shown above in [0168], the chloroTALEN constructs consist of 2 TALEN enzymes, each one recognizing a sequence in the chloroplast genome such that Fok1 nuclease digestion occurs between the 2 TALE binding sites. TALE DNA binding repeats that recognize a specific sequence of interest can be designed using standard methods, for example, and on-line software tool such as http://bao.rice.edu/Research/BioinformaticTools/assembleTALSequences.html. The input to the software tool is a DNA target sequence, in this case, the targeted chloroplast gene sequences. The TALEN enzymes carry a chloroplast transit peptide at the N-terminus, to direct the protein to the chloroplast compartment. The chloroTALEN genes are driven by strong constitutive nuclear promoters, for example, maize and rice Uibiquitin gene promoters. An example of an Agrobacterium T-DNA vector carrying a pair of chloroTALEN cassettes between the Left and Right Border elements required for transfer into the nuclear genome of plants is shown in FIG. 14. In total, 6 chloroTALEN constructs were created, to recognize 2 sites in each of the maize chloroplast psaA, psaB and rbcL genes.

### Example 13: ChloroTALENs Designed Against Specific Sequences in the Chloroplast Genome

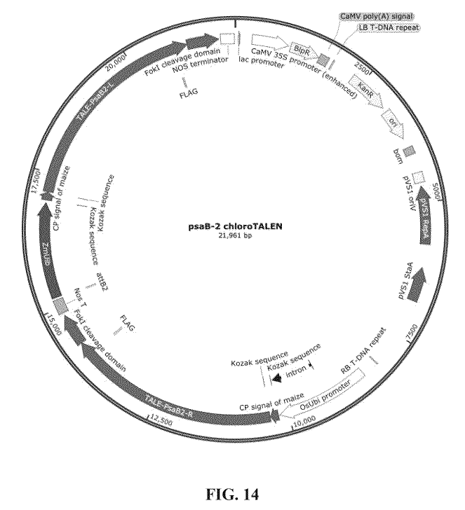

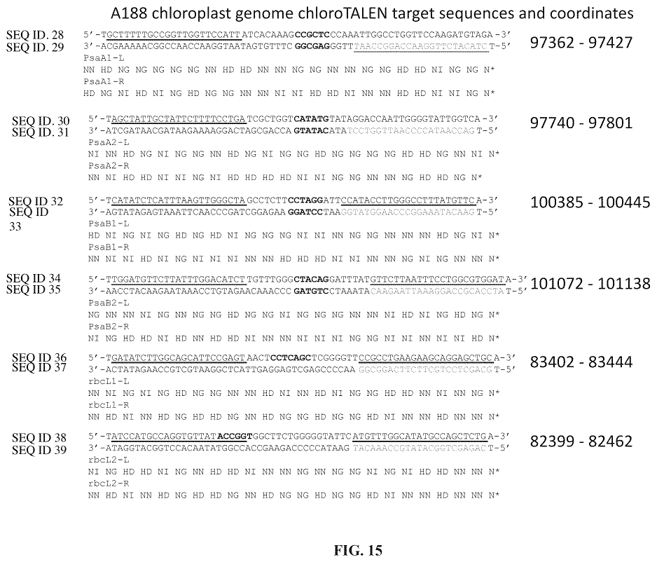

FIG. 15 lists the TALEN target recognition sequences in the maize chloroplast genome for the 6 chloroTALEN constructs. It should be noted, the TALE recognition sequences were chosen to have 100% sequence identity to sorghum and rice, and other monocots. The underlined sequences show the left and right recognition sequences, about 21-23 nucleotides long each with a spacer region of about 18-20 nucleotides where the FokI nuclease is expected to cut. Note that spacer regions were chosen such that a fortuitous restriction enzyme site is present that can be used to monitor nuclease digestion in transgenic plants.

Each nucleotide in the chloroplast recognition sequence is recognized by one of the TALE repeats, that differ only by the 2 amino acids, termed the repeat variable domain (RVD), shown underneath the sequence that provides specificity to the specific TALE repeat, as described above in

### Example 14: Screening for Pigment Mutants in Transgenic Maize Lines Expressing Chloroplast-Targeted TALENs

Stable maize transgenic plants lines were generated via Agrobacterium-mediated transformation of the 6 vectors carrying chloroplast-targeted TALEN enzymes. Selection of transformants was for bialaphos resistance encoded by the BlpR gene shown in FIG. 14. In some cases, identical vectors carrying a nptII gene for paromomycin selection of nuclear transformants were used. Multiple genotypes of maize were used for transformation, including transgenic plants that already carry BBM and WUS genes to enable subsequent recovery of albino leaf sectors as embryogenic callus, as needed.

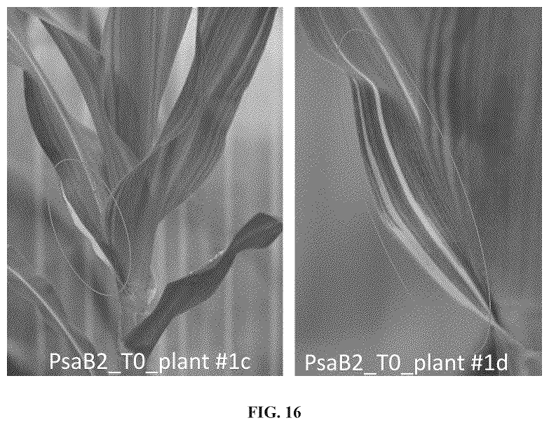

TO transgenic maize plants were grown in tissue culture until rooted and then transferred to soil. In some cases, albino leaf sectors were observed on the TO plants as illustrated and exemplified in FIG. 16 for TO plants derived from transformation with a chloroTALEN construct that targets the psaB gene. Albino sectors indicates a knock-out of photosynthesis, as predicted if the chloroTALENs created the expected nuclease cut at their recognition sequences in the psaA, psaB or rbcl chloroplast gene followed by imperfect resealing of the DNA strands to create a small indel or larger deletion or rearrangement.

### Example 15: Purification of Albino Mutant Leaf Tissue Using Morphogenic Genes

TO transgenic plants that have albino leaf sectors as shown in FIG. 16 may or may not transmit the mutant phenotype to T1 progeny. A more rapid and assured way to recover chloroplast mutant sectors in a purified form, is to introduce the chloroTALEN constructs into a BBM/WUS transgenic genetic background. Since leaf tissue can become embryogenic when placed onto growth media containing the proper plant growth hormones, albino leaf sectors can be recovered in sterile tissue culture via embryogenesis.

TO leaf tissue from greenhouse grown plants (in the BBM/WUS genetic background) that carry albino sectors is dissected to eliminate any neighboring green tissue. The dissected leaf tissue is gently sterilized using a 5% bleach solution. Sterilized albino leaf tissue is then cut into small pieces and placed onto plant growth media with cytokinins and auxins at the appropriate concentrations to stimulate embryogenesis. Embryogenic callus forms within about 2 weeks from the leaf tissue and can be proliferated en mass for subsequent re-transformation of the mutant chloroplasts with the complementing wild-type DNA. In this case, selection of chloroplast transformants is via greening and photosynthetic competence as described above.

### Example 16. Segregation of the Nuclear Transgenic TALEN Constructs from the Chloroplast Non-Photosynthetic Mutant

Once the albino or pigment deficient chloroplast mutant line is established, it may be desirable to segregate away by breeding the nuclear transgenic chloroTALEN construct. Since the TO chloroTALEN transgenic plants are hemizygous, and the chloroplast mutation is maternally inherited, the nuclear transgenes are able to be segregated away in progeny seeds by a simple outcross to wild-type non-transformed plants, used as pollen parent. In this case, T1 progeny are segregating plants that lack a nuclear transgene but carry the chloroplast mutations.

### Example 17. Purification of Mutant Lines to Homoplasmy in Monocots and Dicots is Possible Via Multiple Morphogenic Genes

BBM+WUS over-expression in monocots enables embryogenesis and plant regeneration from leaf-base cells. Additional morphogenic genes have also recently been shown to enable embryogenesis in both monocot and dicot plant species. For example, ZmGRF5-likel and 2 increased transgenic embryogenic callus formation (A188) and increased proliferation of callus (Kong et al 2020) whereas overexpression of the WOX5 gene drastically promoted de novo shoot regeneration from callus (Lee et al. 2022). Likewise, GRF4-GIF1 overexpression increased the frequency of regeneration of transgenic wheat embryos from callus by 8-fold (Debernardi et al. 2020). Similarly, in dicots, overexpression of AtGRF5, BnGRF5-like, AtGRF6 or AtGRF9 resulted in increased transgenic sectors in developing callus and in soybean more meristem initials were observed at axillary nodes from overexpression of GFR5 and GmGRF5-like (Kong et al. 2020). While demonstration of de novo shoot regeneration from other tissues yet needs to be shown, these examples indicates that purification of homoplasmic chloroplast lines can be purified from various tissues in both monocots and dicots using several different morphogenic gene options.

### Example 18. TALE-Cytosine Deaminase-Based Mutation of Chloroplast-Encoded rbcL and atpB Genes

The chloroplast rbcL coding region in the maize A188 genotype was scanned for sites where a cytosine (C) nucleotides is adjacent to a 5′-thymidine nucleotide (5′-CT), that when converted via cytosine deaminase activity to thymine would create a TAG stop codon. As shown in FIG. 17A, conversion of C to T at rbcL amino acid 52 (position 57002 in the maize A188 chloroplast genome, Genbank accession KF241980, https://www.ncbi.nlm.nih.gov/nuccore/KF241980.1/) would convert a glutamine codon to a stop codon (Q52*). This site was chosen as a target for a chloroTALE-cytosine deaminase enzyme since it is close to the intergenic region between the rbcL gene and the neighboring convergent atpB gene, where a cointegrating gene-of-interest could be targeted during complementation of the non-photosynthetic phenotype resulting from the rbcL stop codon mutation.

Two 23 nucleotide long TALE target sequences were identified, separated by a spacer of 19 nucleotides within which the C targeted for mutation is located. TALE repeat sequences (silver arrows, FIG. 17B) were designed, synthesized and cloned into chimeric genes carrying TALE-DddA-UGI scaffolds. The chimeric genes were expressed from either the maize or rice ubiquitin promoter, the nos terminator and are targeted to chloroplasts via the Rubisco small subunit transit peptide (CTP) (FIG. 17B).

The two TALE-DddA-UGI (targeting the left and right rbcL TALE binding sites; SEQ ID NO: 13 and SEQ ID NO:14) were cloned into a pCAMBIA-based vector carrying a bialaphos plant selectable marker gene, to create plasmid PTS424. PTS424 carries T-DNA borders for transfer of sequences into the plant nuclear genome after infection with Agrobacterium.

Transgenic maize expressing BBM and WUS transgenes in the nuclear genome (termed Superline) were used as recipient for re-transformation with the chloroTALE-DddA-UGI construct to enable the potential for subsequent purification of mutant leaf sectors via leaf cell embryogenesis enabled by the BBM/WUS transgenes. Superline maize are readily able to form embryogenic or organogenic callus from leaf-base on medium carrying appropriate plant growth regulators and can be used to regenerate plants from leaf base sectors.

Immature embryos from greenhouse grown Superline plants were transformed with Agrobacterium carrying the PTS424 plasmid as described above. Nuclear retransformed plants were selected on callus induction media with 5 mg/L bialaphos, with subculturing to fresh medium every 2 weeks until resistant microcalli were observed. Once microcalli were observed after ˜6 weeks, bialaphos concentration was increased to 10 mg/L to eliminate false positive calli. Independent calli colonies were split, with half of each colony maintained in the dark on callus induction media and the other half of each calli transferred to regeneration medium in the light.

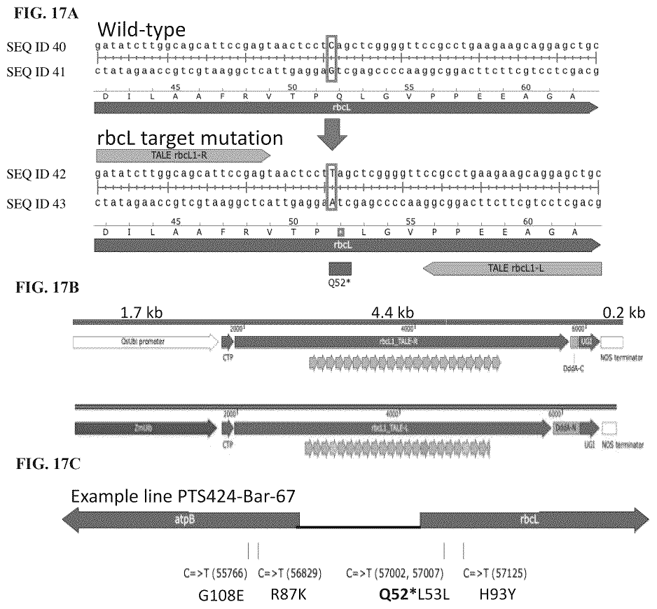

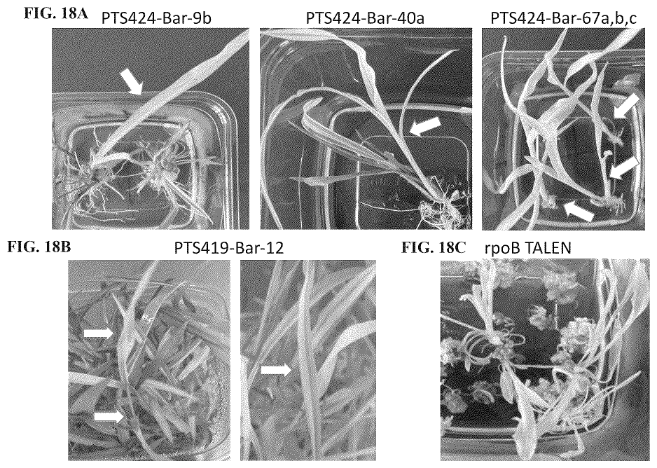

Superline immature embryos were transformed and numberous bialaphos resistant calli were identified with a pigment deficient phenotype. Lines PTS424-Bar-9b, PTS424-Bar-40a and PTS424-Bar-67 are independent lines each identified in tissue culture with a similar strong pigment deficient phenotype as shown in FIG. 18A. Multiple plants regenerated from each independent bialaphos resistant callus and had a mixture of green plants, completely bleached and yellow-colored plants (arrows in FIG. 18A) and plants with both green and yellow sectors. Yellow sectors and plants are indicative of the non-photosynthetic mutation and suggest the targeted chloroplast gene was mutated.

PCR was used to amplify the chloroplast genomic region surrounding the intended rbcL chloroTALE-DddA-UGI target site. The PCR fragment was purified via standard procedures using the XYZ kit (manufacturer) and sequencing of the PCR fragment was performed by Eurofins Genomics (St. Louis). Analysis of the PCR-sequence indicated that all 3 lines, PTS424-Bar-9b, PTS424-Bar-40a and PTS424-Bar-67 carried the intended C→T mutation, creating a stop codon (Q52*) in the rbcL gene. In all cases, the PCR-sequencing result showed a mixture of mutant and wild-type alleles at each position, indicating that the plants were still heteroplasmic for the chloroplast mutation(s). Notably, off-target C [symbol] T mutations were also observed near to the intended rbcL stop codon mutation.

To purify the chloroplast mutations to homoplasmy, we took advantage of the BBM/WUS genetic background to generate embryogenic callus from leaf-base tissue dissected from yellow pigment mutants of each PTS424-Bar line. Embryogenic callus was initiated on callus induction media (CIM), amplified by transfer to new media every 2 weeks for several weeks, and then used to subsequently regenerate plants to test for homoplasmy of the mutations. In this second round of plant regeneration, which is uniquely made possible in maize via the morphogenic BBM/WUS genes, all regenerated plants were observed to be completely yellow pigment mutants (for example, FIG. 18A line PTS424-Bar-67a, b, c lines).

Leaf tissue from these second-round regenerated plants was used for whole chloroplast genome sequencing via the Illumina high throughput sequencing platform (Novogene). Using this approach, the presence of any intended or off-target mutations in the chloroplast genome could be assessed, along with their state of homoplasmy. As shown in Table 4, below, and FIG. 17C, 5 chloroplast C [symbol] T mutations were observed in the PTS424-Bar-67 lines, including 4 mutations in the rbcL gene and 1 mutation in the adjacent atpB gene. All 5 mutations appeared in all next-gen sequence reads, indicating this line is homoplasmic for those 5 mutations. Line PTS424-Bar-40a carried similar mutations as the PTS424-Bar-67 line and additional off-target mutations, but was heteroplasmic and necessitates another round of plant regeneration from leaf-base tissue to further purify the chloroplast mutations in the line. Due to its homoplasmic state, line PTS424-Bar-67 was used for chloroplast and nuclear complementation experiments.

TABLE 4
Chloroplast C [symbol] T mutations

| Mutant | Mapped mutation chloroplst genome position | |
|---|---|---|
| Line | (gene, amino acid location and change) | Mutations |
| [symbol] | 55785(atpB GLOBE), 5582 [symbol] (atpB [symbol] 87K), S7002 (rb [symbol] QS2*) | C=>T |
| | 57037 ( [symbol] cLL [symbol] ), 57325 [symbol] bcl H93Y) | |
| [symbol] | 40,479 ( [symbol] H [symbol] Y), 42,323 (psa 8. [symbol] ), 41.285 (ps [symbol] 525N), | C=>T |
| | 41.522 (psa [symbol] [symbol] 225), 41.862 (psa [symbol] 575 [symbol] ) | |
| r [symbol] TALEN | 29407 (rpo8 T [symbol] 23) | T insertion |

[symbol]

indicates data missing or illegible when filed

### Example 19: Nuclear Complementation of the rbcL/atpB Chloroplast Non-Photosynthetic Mutant Lines

Leaf-derived embryogenic callus of the homoplasmic chloroplast mutant PTS424-Bar-67 line was amplified in tissue culture in the dark on CIM media, with routine transfers to new media every 2 weeks, to bulk up material for Agrobacterium-mediated nuclear transformation. In addition, yellow mutant plants were regenerated from PTS424-Bar-67 embryogenic callus, to enable dissection of leaf-base segments for transformation.

Vectors for nuclear complementation of the rbcL and atpB mutations, PTS438 and PTS444, respectively, were created. The coding region of the chloroplast rbcL and atpB genes were first codon-optimized for maize nuclear expression utilizing the on-line Codon Optimizer Tool from www.IDTDNA.com. For targeting the proteins to chloroplasts, the chloroplast transit peptide from the maize EPSP Synthase gene (GenBank: AEP17820.1) was fused in-frame to the N-terminus of the rbcL and atpB coding regions. The CTP-rbcL and CTP-atpB genes were synthesized (ThermoFisher) and cloned between a variant of the maize Ubiquitin 1 promoter and nos terminator (SEQ ID NO:15 and SEQ ID NO:16) allowing for constitutive expression in maize. The PTS438 and PTS444 vectors also carry a hygromycin antibiotic resistance gene driven by the CaMV 35 enhanced promoter and 35S polyA termination signals, for selection of nuclear transformed plants. The CTP-rbcL or CTP-atpB and hygromycin resistance genes are located between Left- and Right-T-DNA border sequences in an Agrobacterium vector for transfer into the maize nuclear genome.

Agrobacterium-mediated transformation of embryogenic callus or leaf-base sections was performed according to standard protocols. After co-cultivation of Agrobacterium, callus or leaf base sections were cultured on CIM media with 50 mg/L hygromycin in the dark to begin select for nuclear transformants. Microcalli were transferred to fresh media every 2 weeks for several transfers until only rapidly growing independent calli were isolated. After 1-2 additional media transfers, independent hygromycin resistant calli were transferred to the light on plant regeneration medium to identify green regenerated plants that indicate nuclear complementation of the chloroplast mutants had occurred.

Since the PTS424-Bar-67 line carries chloroplast mutations in both the rbcL and atpB genes, it was important to determine if the nuclear complementing CTP-rbcL or the CTP-atpB, or both genes, are required for the green plant phenotype. Therefore, Agrobacterium strains carry either transgene were transformed separately into PTS424-Bar-67 tissues or the Agrobacterium strains were combined in a 1:1 ratio and co-cultivated together with PTS424-Bar-67 tissues. Green calli and regenerating plants are confirmed as nuclear transgenic via the presence of both the nuclear hygromycin resistance and the CTP-rbcL or CTP-atpB genes, while in the same samples the chloroplast encoded mutations are confirmed via PCR-sequencing as described above, proving that nuclear-encoded chloroplast mutant complementation had occurred. In subsequent experiments, variants of the rbcL and atpB genes can be used to test for enhancements in chloroplast Rubisco or ATPase function and improved photosynthetic parameters, including improved yield.

### Example 20. Chloroplast Complementation of the Chloroplast rbcL/atpB Non-Photosynthetic Mutant Lines

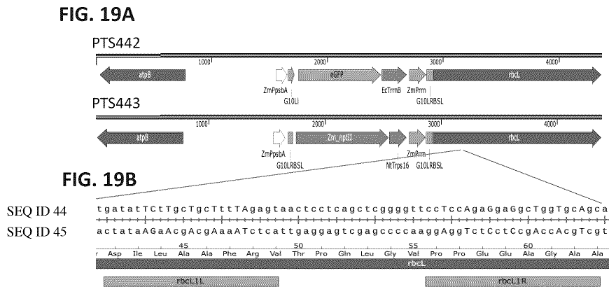

Embryogenic callus and leaf-base tissue derived from the PTS424-Bar-67 mutant line is also used for complementation via chloroplast transformation. In this case, chloroplast transformation vectors PTS442 and PTS443 were created (FIG. 19). In each vector, complementing DNA includes ˜3 kb of the chloroplast genome region that includes the oppositely oriented rbcL and atpB genes along with the intergenic region between the genes that carries their promoter regions. The complementing rbcL and atpB gene sequences carry the wild-type allele at each of the 5 C [symbol] T mutation sites, to restore full photosynthetic ability to complemented lines (FIG. 19A). Additionally, the chloroTALE-DddA-UGI target sites in the rbcL coding region have been mutated at several positions (FIG. 19B) to prevent binding of the deaminase enzyme to the target site, thus preventing mutations to reoccur in complemented chloroplast genomes. Since the chloroTALE-DddA-UGI target sites are located in the coding region of the rbcL gene, target site mutations were chosen in the 3rd position of each codon, to disrupt the target site but not alter the rbcL coding amino acid sequence.

In PTS442, a chimeric GFP gene is included in the intergenic region between the rbcL and atpB coding regions, to facilitate early detection of chloroplast transformed cells. The GFP transgene is expressed from the maize chloroplast psbA gene promoter (ZmPpsbA) with the translation control region from the bacteriophage T7 gene 10 leader (G10L) and carries a 3′-transcript termination sequence from the E. coli rrnB gene (EcTrrnB). To ensure that integration of the chimeric GFP transgene or any subsequent transgenes have not affected expression of the chloroplast rbcL gene, a new transgenic promoter (maize chloroplast Prrn promoter, ZmPrrn, with the G10L) has been placed in front of the rbcL coding region to ensure its expression in complemented chloroplasts. PTS443 is analogous to PTS442, except that a chimeric nptII gene is cloned into the atpB/rbcL intergenic region, to enable selection for resistance to the antibiotics kanamycin, paromomycin or neomycin in transformed chloroplasts.

### Example 21. TALE-Cytosine Deaminase-Based Mutation of Chloroplast-Encoded psaA and psaB Genes

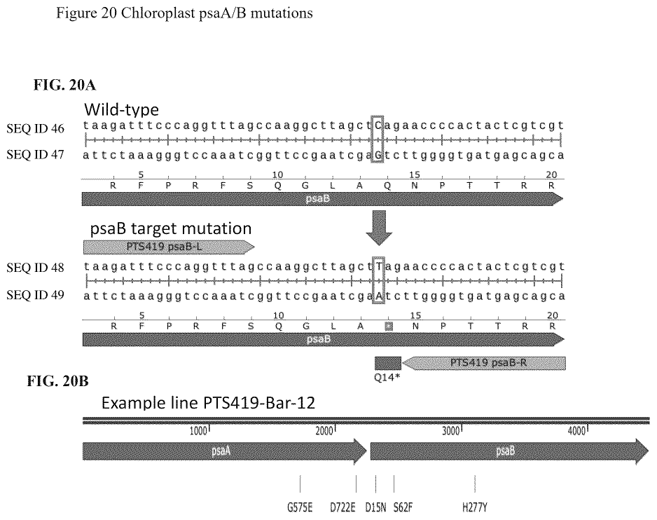

The chloroplast psaA and psaB are co-transcribed and co-translationally coupled (reference) so their coding regions are separated by only 25 nucleotides in maize. Disruption of the function of either one of those genes may cause a non-photosynthetic phenotype, making them good candidates for creating pigment mutant plant lines. The downstream psaB gene was scanned for a C [symbol] T mutation that would create a stop codon in an N-terminal portion of the coding region. Mutation of glutamine codon at psaB amino acid 14 (FIG. 20A) was chosen (Q14*).

TALE target sequences of 19 nucleotides (left TALE site) and 18 nucleotides (right TALE site) were identified, separated by a spacer of 16 nucleotides within which the Q14* site occurs. TALE repeat sequences were designed, synthesized and cloned into chimeric genes carrying TALE-DddA-UGI scaffolds (SEQ ID NO:17 and SEQ ID NO:18). The chimeric genes were expressed from either the maize or rice ubiquitin promoter and targeted to chloroplasts via the Rubisco small subunit transit peptide (CTP). Similar to above, the two TALE-DddA-UGI (targeting the left and right TALE binding sites) were cloned into a pCAMBIA-based vector carrying a bialaphos plant selectable marker gene, to create plasmid PTS419. PTS419 carries T-DNA borders for transfer of sequences into the plant nuclear genome after infection with Agrobacterium.

Maize lines carrying BBM and WUS genes were transformed with Agrobacterium carrying the PTS419 plasmid as described above. After multiple rounds of selection on bialaphos-containing media and subsequent plant regeneration in the light, 1 pale green pigment mutant plant line was observed, termed PTS419-Bar-12 (FIG. 18B). At least one completely pale-green plant and several chimeric pale-green and green striped plants were identified from a single regenerating callus, indicating this line was likely heteroplasmic for chloroplast mutation. PCR amplification and sequencing of the psaA/psaB locus indicated that the Q14* intended mutation did not occur in this plant line, but instead a total of 5 C [symbol] T off-target mutations occurred in the psaA and psaB genes.

Leaf-base cuttings from the PTS419-Bar-12 line were used for another round of embryogenic callus formation and subsequent plant regeneration. Multiple regenerated plants with a completely pale-green phenotype were observed and used for subsequent whole chloroplast genome sequencing. The PTS419-Bar-12 line (including newly regenerated sublines PTS419-Bar-12-6 and PTS419-Bar-12-11) were homoplasmic for the 5 chloroplast genome mutations within the psaA and psaB coding regions.

### Example 22. Nuclear Complementation of the psaA/psaB Chloroplast Non-Photosynthetic Mutant Lines

Vectors for nuclear complementation of the psaA and psaB mutations, PTS445 and PTS437, respectively, were created, similarly to the PTS438 and PTS444 nuclear complementation vectors described above. The coding region of the chloroplast psaA and psaB genes were first codon-optimized for maize nuclear expression utilizing the on-line Codon Optimizer Tool from www.IDTDNA.com, and the chloroplast transit peptide from the maize EPSP Synthase gene (GenBank: AEP17820.1) was fused in-frame to the N-terminus of the coding regions. The transgenes were expressed from the maize Ubiquitin 1 promoter and the nos terminator sequence (SEQ ID NO: 19 and SEQ ID NO:20) and cloned into the Agrobacterium T-DNA vector carrying the hygromycin antibiotic resistance gene.

Agrobacterium-mediated transformation of embryogenic callus or leaf-base sections was performed according to standard protocols. After co-cultivation of Agrobacterium, callus or leaf base sections were cultured on CIM media with 50 mg/L hygromycin in the dark to to begin select for nuclear transformants. Microcalli were transferred to fresh media every 2 weeks for several transfers until only rapidly growing independent calli were isolated. After 1-2 additional media transfers, independent hygromycin resistant calli were transferred to the light on plant regeneration medium to identify green regenerated plants that indicate nuclear complementation of the chloroplast mutants had occurred.

Since the PTS419-Bar-12 line carries chloroplast mutations in both the psaA and psabB genes, it was important to determine if one or both of the nuclear complementing genes are required for the green plant phenotype. Therefore, Agrobacterium strains carry either transgene were transformed separately into PTS419-Bar-12 tissues and the Agrobacterium strains were combined in a 1:1 ratio and co-cultivated together with PTS424-Bar-12 tissues. Fully green regenerating plants are confirmed as nuclear transgenic via the presence of the nuclear hygromycin resistance and the CTP-psaA or CTP-psaB genes, while in the same samples the chloroplast encoded mutations are confirmed via PCR-sequencing as described above, proving that nuclear-encoded chloroplast mutant complementation had occurred.

### Example 23. Chloroplast Complementation of the Chloroplast psaA/psaB Non-Photosynthetic Mutant Lines

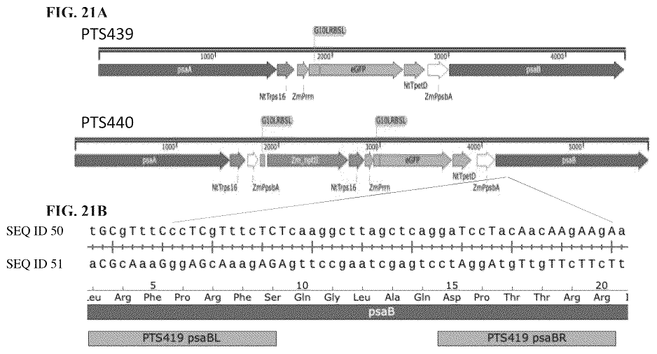

Embryogenic callus and leaf-base tissue derived from the PTS419-Bar-12 mutant line is also used for complementation via chloroplast transformation. In this case, chloroplast transformation vectors PTS439 and PTS440 were created. In each vector, complementing DNA includes ˜3 kb of the chloroplast genome region that includes nearly all of the psaA and psaB coding regions. The psaA and psaB coding regions carry the wild-type allele at each of the 5 C [symbol] T mutation sites, to restore full photosynthetic ability to complemented lines. Additionally, the chloroTALE-DddA-UGI target sites have been mutated at several positions to prevent binding of the deaminase enzyme to the target site, thus preventing mutations to reoccur. Since the chloroTALE-DddA-UGI target sites are located in the coding region of the psaB coding region, target site mutations were chosen in the 3rd position of each codon, to disrupt the target site but not alter the amino acid sequence (FIG. 21).

In PTS439, a chimeric GFP gene is included in the intergenic region between the psaA and psaB coding regions, to facilitate early detection of chloroplast transformed cells. The GFP transgene is expressed from the maize chloroplast PrmnGIOL promoter/leader sequence and carries a 3′-transcript termination sequence from the tobacco chloroplast petD gene. Since the 25 nucleotide intergenic sequences are disrupted in PTS439, a new 3′-end has been added to the psaA gene, from the tobacco chloroplast rps16 gene, and a new promoter added to the psaB gene, from the maize chloroplast psbA gene. PTS440 is identical to PTS439, except that a chimeric nptII gene driven by the maize psbA promoter and tobacco rps16 gene 3′-end is cloned between the psaA and GFP genes to enable selection for resistance to the antibiotics kanamycin, paromomycin or neomycin in transformed chloroplasts.

### Example 23. Identification of a Homoplasmic Chloroplast rpoB Gene Mutant from TALEN-Mediated Transformation

During screening for pigment mutant plant lines after transformation of a BBM+WUS nuclear transgenic maize line with Agrobacterium carrying the vector with chloroTALENs transgenes targeting the rbcL1 site (FIG. 15), a unique albino regenerated plant was observed, termed rpoB TALEN (FIG. 18C). Embryogenic callus was established from leaf base of this line and amplified in tissue culture in the dark. Screening of this line via PCR sequencing of the intended rbcL locus did not identify any mutations in the rbcL coding region or surrounding genomic sequences.

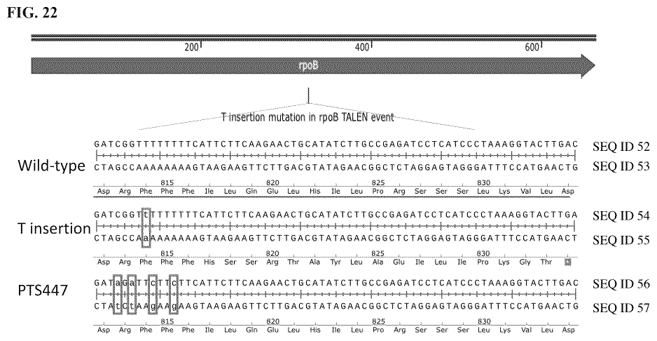

An additional 2 rounds of albino plant regeneration from leaf base-derived callus was performed to ensure homoplasmy of any chloroplast genome mutations. Callus from the rpoB TALEN regenerated subclones was then used for whole chloroplast genome sequencing via Novogene high throughput sequencing and SNP detection platform. From this sequencing analysis, a single nucleotide insertion in the chloroplast rpoB coding region was identified. The insertion of a T (thymidine) after amino acid 813 (Table 4) creates a frameshift mutation that creates a new stop codon at amino acid 834 (FIG. 22), resulting in a truncated and non-functional rpoB protein that is normally 1527 amino acids long. The rpoB gene encodes a critical subunit of the Plastid-encoded RNA Polymerase (PEP) enzyme responsible for transcription of chloroplast photosynthetic genes (citation). Therefore, this rpoB mutant is expected to be deficient in expression of numerous chloroplast-encoded photosynthetic genes, explaining its albino phenotype.

### Example 24. Nuclear Complementation of the rpoB Mutant Line

A nuclear transformation vector based on Agrobacterium T-DNA is created for nuclear complementation of the chloroplast rpoB mutant line in a similar as above. The 1527 amino acid coding region of rpoB was codon optimized, synthesized and cloned with an N-terminal fusion to the maize EPSPS CTP, and driven by the maize ubiquitin 1 promoter and the nos terminator (SEQ ID NO: 21), adjacent to the hygromycin resistance gene, to create PTS446. After Agrobacterium transformation of rpoB mutant line callus, selection for green regenerating plants and/or hygromycin resistance identifies nuclear complemented lines.

### Example 25. Chloroplast Complementation of the rpoB Mutant Line

The rpoB insertion mutation lies in the middle of the ˜3.2 kb gene, making complementation of the single nucleotide mutation along with the concomitant insertion of a desired passenger transgene into a distal intergenic region difficult. To circumvent this complication, we are using a co-transformation approach whereby complementation of the rpoB mutation is achieved using plastid PTS447 that carries a 662 bp fragment of the rpoB gene surrounding the T insertion mutation (SEQ ID NO: 22), and a second plasmid carrying a desired transgene and/or a selectable marker gene targeted to an intergenic region in a different location of the maize chloroplast genome. The complementing rpoB gene fragment in PTS447 removes the extra T that causes the frameshift mutation in the gene, and carries 4 new nucleotide changes at each of the neighboring 4 amino acids, with the changes placed in the silent 3^{rd} position of those codons such that no amino acid changes occur (FIG. 22). These nucleotide changes are easily identified via sequencing of the rpoB gene region in green complemented lines and are used to prove that chloroplast complementation has occurred. To facilitate identification of cotransformed lines, a selectable antibiotic or herbicide resistance marker can be carried on the cotransforming plasmid, or a fluorescent reporter gene such as GFP can be used for early visual detection.

Although the present disclosure has been described with reference to preferred embodiments, workers skilled in the art will recognize that changes may be made in form and detail without departing from the spirit and scope of the disclosure.

What is claimed is:

1. A method for transforming a plant with a heterologous DNA molecule comprising:
(a) introducing a heterologous DNA molecule into (i) a recipient homoplasmic non-photosynthetic plant cell comprising plastids with a mutation in a plastid photosynthetic gene DNA sequence to obtain a transformed plant cell containing the heterologous DNA molecule, wherein the heterologous DNA molecule comprises a promoter, a DNA encoding a chloroplast transit peptide (CTP) and a protein having an enzymatic and/or biological activity of a wild-type protein encoded by the wild-type plastid photosynthetic gene DNA sequence, wherein the promoter, DNA encoding the CTP and protein are operably linked;
(b) exposing the transformed plant cell from step (a) to light sufficient to support greening of a photosynthetic plant cell; and,
(c) selecting or screening for a green photosynthetic plant cell comprising a transformed plant containing a nuclear genome comprising the heterologous DNA molecule from the plant cells exposed to the light in step (b), thereby transforming a plant with a heterologous DNA molecule.

2. The method of claim 1, further comprising the step of obtaining a transgenic plant comprising the nuclear genome from the green photosynthetic plant cell of step (c).

3. The method of claim 1, wherein the heterologous DNA molecule further comprises a DNA molecule that confers an agronomically beneficial trait, a desirable non-agronomic trait, or combination thereof.

4. The method of claim 1, wherein the protein having an enzymatic and/or biological activity of a wild-type protein encoded by the wild-type plastid photosynthetic gene DNA sequence is not the wild-type protein encoded by the wild-type plastid photosynthetic gene DNA sequence.

5. The method of claim 1, wherein the protein having an enzymatic and/or biological activity of a wild-type protein encoded by the wild-type plastid photosynthetic gene DNA sequence provides for improved photosynthesis, an increased accumulation of the protein encoded by the photosynthetic gene, a decrease in the Km of the protein encoded by the photosynthetic gene for a substrate of the protein, an increase in the Kcat of the protein encoded by the photosynthetic gene, or any combination thereof.

6. The method of claim 1, wherein the recipient homoplasmic non-photosynthetic plant cells in step (a) are grown in culture as callus, embryogenic callus, organogenic cultures, suspension cells or leaf cells or protoplasts and wherein the green photosynthetic plant cell of step (c) obtained from the non-photosynthetic plant cells grown in culture is used to regenerate a transgenic plant.

7. The method of claim 1, wherein the selecting of plant cells in step (b) comprises selecting a sector of green plant cells.

8. The method of claim 1, wherein a selectable antibiotic resistance-or herbicide tolerance conferring gene is not introduced or not used for selection in the recipient plant cell in step (a).

9. The method of claim 1, wherein the plant cell is not a tobacco or Arabidopsis plant cell.

10. The method of claim 1, wherein the plant cell is a monocot plant cell or wherein the plant cell is a maize, sorghum, wheat, or rice plant cell.

11. The method of claim 1, wherein the plant cell is a dicot plant cell or wherein the plant cell is a cotton, soybean, Brassica sp., potato, or tomato plant cell.
